(19) United States
(12) Patent Application Publication
(10) Pub. No.: US 2005/0020544 A1
(43) Pub. Date: Jan. 27, 2005

(54) Bicyclic CB2 cannabinoid receptor ligands

(76) Inventors: Aaron Garzon, Rehovot (IL); George Fink, Tel Aviv (IL); Dalit Esther Dar, Kiryat Yam (IL); Naim Menashe, Nes Ziona (IL); Ayelet Nudelman, Rehovot (IL); Orit Greenberg, Petah Tikva (IL); Avihai Yacovan, Gedera (IL)

Correspondence Address: WINSTON & STRAWN;PATENT DEPARTMENT, 1400 L STREET, N.W., WASHINGTON, DC, 20005-3502, US

(21) Appl. No.: 10/895,759

(22) Filed: Jul. 20, 2004

Related U.S. Application Data
(63) Continuation of application No. PCT/IL03/00077, filed on Jan. 30, 2003.

(30) Foreign Application Priority Data
Jan. 31, 2002 (IL) IL 147941
May 2, 2002 (WO) PCT/IL02/00341
Jun. 18, 2002 (IL) IL 150302

(51) Int. Cl. C07F 9/28; A61K 31/66; A61K 31/255; A61K 31/195

(52) U.S. Cl. 514/114; 514/143; 514/125; 514/518; 514/567; 514/568; 562/11; 562/45; 562/459

(57) ABSTRACT

The present invention relates to non-classical cannabinoids that are ligands of the peripheral cannabinoid receptor CB2, and to pharmaceutical compositions thereof comprising as an active ingredient novel (+) alpha-pinene derivatives, which are useful for prevention and treatment of autoimmune diseases including but not limited to rheumatoid arthritis, multiple sclerosis, systemic lupus erythematosus, myasthenia gravis, diabetes mellitus type I, hepatitis, psoriasis, tissue rejection in organ transplants, malabsorption syndromes such as celiac disease, pulmonary diseases such as asthma and Sjögren's syndrome, inflammation including inflammatory bowel disease, pain including peripheral, visceral, neurophathic inflammatory and referred pain, muscle spasticity, cardiovascular disorders including arrhythmia, hypertension and myocardial ischemia, neurological disorders including stroke, migraine and cluster headaches, neurodegenerative diseases including Parkinson's disease, Alzheimer's disease, amyotrophic lateral sclerosis, Huntington's chorea, prion-associated neurodegeneration, CNS poisoning and certain types of cancer.

### CROSS-REFERENCE TO RELATED APPLICATIONS

This application is a continuation of International Application PCT/IL03/00077, filed Jan. 30, 2003, the content of which is expressly incorporated herein by reference thereto.

### FIELD OF THE INVENTION

The present invention relates to (+) α-pinene derivatives that are ligands of the peripheral cannabinoid receptor CB2, and to pharmaceutical compositions thereof, which are useful for prevention and treatment of autoimmune diseases and related disorders, inflammation, pain, muscle spasticity, cardiovascular disorders, neurological disorders, neurodegenerative diseases, CNS poisoning and certain types of cancer.

### BACKGROUND OF THE INVENTION

Cannabis sativa preparations have long been known as therapeutic agents to treat various diseases (Mechoulam, R in “Cannabinoids as Therapeutic Agents” CRC Press, Boca Raton, Fla., 1-19, 1986). The native active constituent, Delta 9-tetrahydrocannabinol (Δ⁹-THC), is prescribed today, under the generic name Dronabinol, as an anti-emetic and for enhancement of appetite, mainly in AIDS patients. However, separation between the clinically undesirable psychotropic effects and the therapeutically desirable effects, such as vascular hypotension and immunomodulation, has only recently been accomplished. The discovery of two cannabinoid receptors, CB1 and CB2, has helped to elucidate the diverse cannabinoid effects.

The receptors were shown to have seven transmembrane structures G-protein coupled that share 44% amino acid sequence homology but differ in tissue specificity (Munro, S., Thomas, K. L. & Abu-Shaar, M., Nature 365: 61-5, 1993). Both receptors exert their effect by negative regulation of adenylyl cyclase activity through the pertussis toxin-sensitive GTP-binding protein. They were also shown to activate the mitogen activated protein kinase (MAPK) in certain cell types (Parolaro, D., Life Sci. 65: 637-44, 1999).

The CB1 receptor is expressed mainly in the CNS and to a lesser extent in other tissues. CB1 receptors are primarily found in brain regions associated with the behavioral effects of cannabinoids, such as the hippocampus, amygdala, cortex, basal ganglia and cerebellum. Furthermore, elevated levels of CB1 receptors are found in areas that modulate nociceptive processing. The CB2 receptor is expressed mostly in peripheral tissue associated with immune functions, including macrophages, B and T cells, as well as in peripheral nerve terminals and on mast cells (Pertwee, R. G., Prog. Neurobiol. 63: 569-611, 2001). While the effects mediated by CB1, primarily in the CNS, have been thoroughly investigated those mediated by CB2 are only now being elucidated.

The neuroanatomical distribution of the receptors was determined using radiolabeled THC analogs such as [³H]CP-55940 (Elphick, M. R. & Egertova, M., Phil. Trans. R. Soc. Lond. B Biol. Sci. 356: 381-408, 2001). Highest concentrations of cannabinoid binding site, specifically CB1 receptor, are found in the basal ganglia and cerebellum, regions of the brain that are involved in movement. A subpopulation of the receptors is expressed in the peripheral terminals of the dorsal root ganglion. It has been suggested that the analgesic effects of cannabinoids are mediated at anatomically distinct sites than for their motor effects. Additional techniques, including immunohistochemistry, in situ hybridization assays using specific transcripts (Galiegue, S. et al., Eur. J. Biochem. 232: 54-61, 1995) and knockout mice (Buckley, N. E. et al., Eur. J. Pharmacol. 396: 141-9, 2000) have been used to contribute to the understanding of the receptors' expression patterns and function. The CB2 receptor is not expressed in the brain but is particularly abundant in immune tissues, with an expression level 10-100 fold higher than that of CB1. In spleen and tonsils, the CB2 mRNA content was equivalent to that of CB1 mRNA in the central nervous system. Among the main human blood cell subpopulations, the distribution pattern of the CB2 mRNA displayed important variations with higher levels in B-cells than in natural killer cells or monocytes and low levels in polymorphonuclear neutrophil cells, T8 cells and T4 cells.

CB 1 knockout mice have been shown to be unresponsive to cannabinoids in behavioral assays providing molecular evidence that the psychotropic effects, including sedation, hallucinations and delirium and anti-nociception are manifested through activation of the CB1 receptor, present primarily in the CNS. Analysis of the CB2 knockout mouse has corroborated the evidence for the function of CB2 receptors in modulating the immune system. CB2 does not affect immune cell development and differentiation as determined by FACS analysis of cells from the spleen, lymph node and thymus from CB2 knockout mice, but rather mediates the suppressive effect of Δ⁹-THC.

Due to the restricted expression of the CB2 receptor in subsets of immune cells and neurons, selective CB2 ligands have therapeutic value (Pertwee, R. G., Curr. Med. Chem. 6: 635-64, 1999). Of particular interest are those compounds with high affinity and high specificity for the CB2 receptor. These compounds could afford the benefits of CB2 agonism while avoiding the adverse side effects seen in compounds with affinity for the CB1 receptor. Such compounds could be effective in the treatment of autoimmune diseases including but not limited to multiple sclerosis, rheumatoid arthritis, systemic lupus erythematosus, myasthenia gravis, diabetes mellitus type I, hepatitis, inflammatory bowel disease or irritable bowel syndrome, psoriasis and other immune related disorders including but not limited to tissue rejection in organ transplants, malabsorption syndromes such as celiac disease, pulmonary diseases such as asthma and Sjögren's syndrome.

The discovery of cannabinoid receptors and the more recent identification of endocannabinoids, endogenous ligands capable of activating the CB receptors, has led to the understanding of the multiplicity of effects exerted by cannabinoids and related compounds. On top of a general neuroprotective effect of certain cannabinoid agonists more specific applications can be found. Thus, for example, evidence for the tonic control of spasticity by the endocannabinoid system suggests that cannabinoid agonists may help in the treatment of muscle spasm and tremor in multiple sclerosis (Baker D. et al., FASEB J. 15: 300-2, 2001), in addition to the possible moderation of the disease by immunomodulation through an action on CB2 receptors expressed by immune cells. Cannabinoid agonists may also prove to be of help in the treatment muscle spasm in cancer and HIV/AIDS (Hall W. D., Degenhardt L. J. & Currow D., Med. J. Aust. 175: 39-40, 2001 ) and of neuromuscular disorders.

Activation of the CB1 receptor has therapeutic benefits in the treatment of pain and inflammation in addition to the sedative and undesirable psychotropic effects. Cannabinoids not only inhibit acute pain processing through action on nociresponsive neurons but also modulate persistent pain and inflammation-induced behavioral hypersensitivity. In addition to their central effects, cannabinoids also inhibit pain at the site of injury and interestingly the anti-inflammatory and anti-hyperalgesis actions of cannabinoids in the periphery may involve CB2 receptor mediated activity as well. Compounds that selectively activate the CB2 receptor have potential as immunomodulatory agents and may offer a therapeutic approach to treating autoimmune diseases and related disorders. In addition, selective CB2 receptor agonists have been shown to be useful in the treatment of inflammation and pain, myocardial ischemia and certain types of cancer. THC, as well as the two major endogenous ligands identified so far, arachidonoylethanolamide (anandamide or AEA) (Devane, W. A. et al., Science 258: 1946-9, 1992) and 2-arachidonylglycerol (2-AG) (Sugiura, T. et al., Biochem. Biophys. Res. Commun. 215: 89-97, 1995) exert most of their effects by binding to both cannabinoid receptors.

Several synthetic compounds have been shown to bind to the CB2 receptor with a higher affinity than to the CB1 receptor (Pertwee, R. G., Expert Opin. Investig. Drugs 9: 1553-71, 2000). Cannabinoid receptor agonists comprise four main groups of compounds. The classic cannabinoids maintain the dibenzopyran ring system of THC while the non-classical cannabinoids include bicyclic or tricyclic analogs lacking the pyran ring. The aminoalkylindoles and analogs make up the third family and the endocannabinoids including anandamide and other fatty acid derivatives comprise the fourth family. For instance, L-759656 is a classical cannabinoid analog and HU-308 is a bicyclic analog. Both have CB2/CB1 binding affinity ratios of 300-400 and both have been shown to behave as potent and specific CB2 agonists in functional assays (Hanu\check{s}, L. et al., Proc. Natl. Acad. Sci. USA 96: 14228-33, 1999; Ross, R. A. et al., Br. J. Pharmacol. 126: 665-72, 1999).

The evidence linking CB2 receptor activation with therapeutic properties is manifold. The involvement of cannabinoids in cardioprotection, against ischemic and reperfusion effects including arrhythmia specifically through activation of the CB2 has recently been described in PCT patent application WO 01/28588 and by Krylatov et al. (Krylatov A. V. et al., Bull. Exp. Biol. Med. 131: 523-5, 2001), the disclosures of which are hereby incorporated by reference. Cannabinoids may be potential anti-tumoral agents owing to their ability to induce the regression of various types of tumors, including lung adenocarcinoma, glioma, thyroid epithelioma and skin non-melanoma in animal models. Certain tumors, especially gliomas, express CB2 receptors. Guzman et al. (Galve-Roperh, I. et al., Nat. Med. 6: 313-9, 2000; Guzman, M., Sanchez, C., Galve-Roperh, I., J. Mol. Med. 78: 613-25, 2001) have shown that THC and WIN55212-2, the former a natural ligand and the latter a synthetic cannabinoid, induce the regression or eradication of malignant brain tumors in animals. The rat glioma C6 cell line expresses CB2 and on the basis of studies with selective CB antagonists, it has been proposed that activation of either of the receptors may trigger apoptosis.

The role of the endocannabinoid system in immunosuppression is the focus of many studies (Berdyshev, E. V., Chem. Phys. Lipids 108: 169-90, 2000). Anandamide (AEA), Palmitoylethanolamide (PEA) and 2-AG were shown to down-regulate the immune response in a variety of experimental systems and function as anti-inflammatory and imminunosuppressive agents.

THC is known for its analgesic properties. The two major endogenous ligands, AEA and 2-AG have also been shown to act as analgesic agents and can exert their effects by binding to both cannabinoid receptors (Calignano, A. et al., Nature 394: 277-81, 1998). Therefore agonists of the CB2 receptor or putative CB2-like receptors are useful as agents for suppressing peripheral, visceral, neuropathic, inflammatory and referred pain. Moreover, a CB2 receptor ligand may be protective against CNS poisoning.

U.S. Pat. No. 4,208,351 discloses optically active bicyclic compounds as intermediates in a stereoselective process for the preparation of classical tricyclic cannabinoids. However, no therapeutic activity was attributed to the intermediates, no mention was made to the ability of such compounds to bind cannabinoid receptors altogether and thus no pharmaceutical composition comprising such compounds were envisioned.

U.S. Pat. No. 4,282,248 discloses both isomeric mixtures and individual isomers of pinene derivatives. Therapeutic activity, including analgesic, central nervous system depressant, sedative and tranquilizing activity, was attributed to the compounds, but the disclosure did not teach that said compounds would bind to any cannabinoid receptor.

U.S. Pat. No. 5,434,295 discloses a family of novel 4-phenyl pinene derivatives, and teaches how to utilize said compounds in pharmaceutical compositions useful in treating various pathological conditions associated with damage to the central nervous system. This disclosure neither teaches nor suggests that any of those are selective for peripheral cannabinoid receptors. International patent application WO 01/32169 discloses a family of bicyclic compounds, including HU-308, as CB2 specific agonists and exemplifies their use in the treatment of pain and inflammation, autoimmune diseases, gastrointestinal disorders and as hypotensive agents.

U.S. Pat. No. 6,013,648 discloses indole derivatives that are CB2 specific agonists and may be used for preparing immunomodulating drugs. International patent application WO 01/28497 discloses novel bicyclic cannabinoid analogs that exhibit high affinity for the CB2 receptor. It is apparent to the skilled artisan that the compounds in said patent are of a stereochemical orientation wherein C-1, C-4 and C-5 are R, when referring to the nomenclature adopted in the present disclosure. However, the corresponding (+) α-pinene derivatives have not been synthesized and their therapeutic activity is unknown.

It is understood that the present invention explicitly excludes known compounds, including those disclosed in U.S. Pat. Nos. 4,208,351, 4,282,248 and 5,434,295 and in international patent application WO 01/32169; though certain novel properties of these compounds are claimed as such.

### SUMMARY OF THE INVENTION

It is an object of the present invention to provide novel α-pinene derivatives and compositions thereof In particular, preferred compounds display specific binding affinity toward the peripheral cannabinoid receptor CB2, thereby providing methods of treatment comprising specific therapeutic CB2 binding ligands. These methods involve the use of appropriately formulated pharmaceutical compositions. It is another object of the present invention to provide CB2 binding ligands, capable of exerting their CB2 receptor-specific effects in vivo. The present invention further provides methods for preventing and treating diseases by administering to an individual in need thereof of a pharmaceutical composition containing a therapeutically effective amount of a CB2 specific ligand as an active ingredient.

According to a first embodiment of the present invention, we disclose a compound of the general formula (1): 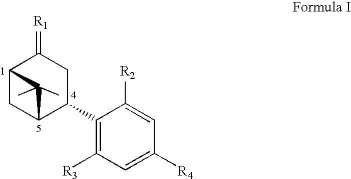
having a specific stereochemistry wherein C-5 is S, the protons at C-1 and C-5 are cis in relation to one another and the protons at C-4 and C-5 are trans; and wherein:
R₁ is selected from the group consisting of
   (a) O or S,
   (b) C(R′)₂ wherein R′ at each occurrence is independently selected from the group consisting of hydrogen, cyano, —OR″, —N(R″)₂, a saturated or unsaturated, linear, branched or cyclic C₁-C₆ alkyl, C₁-C₆ alkyl-OR″ or C₁-C₆ alkyl-N(R″)₂ wherein at each occurrence R″ is independently selected from the group consisting of hydrogen, C(O)R′″, C(O)N(R′″)₂, C(S)R′″, saturated or unsaturated, linear, branched or cyclic C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂ alkyl, and
   (c) NR″ or N—OR″ wherein R″ is as previously defined;
R₂ and R₃ are each independently selected from the group consisting of
   (a) halogen,
   (b) —R″, —OR″, —N(R″)₂, —SR″, —S(O)(O)NR″, wherein at each occurrence R″ is as previously defined,
   (c) —S(O)R^{b}, —S(O)(O)R^{b}, —S(O)(O)OR⁶ wherein R^{b} is selected from the group consisting of hydrogen, saturated or unsaturated, linear, branched or cyclic C₁-C₆ alkyl, C₁-C₆ alkyl-OR″, and C₁-C₆ alkyl-N(R″)₂, wherein R″ is as previously defined, and
   (d) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear, branched or cyclic C₁-C₆ alkyl and R^{e} is at each occurrence selected from the group consisting of hydrogen and R^{d} as previously defined; and
R₄ is selected from the group consisting of
   (a) R wherein R is selected from the group consisting of hydrogen, halogen, OR′″, OC(O)R′″, C(O)OR′″, C(O)R′″, OC(O)OR′″, CN, NO₂, N(R′″)₂, NC(O)R′″, NC(O)OR′″, C(O)N(R′″)₂, NC(O)N(R′″)₂, SR′″, and C(S)R′″, wherein at each occurrence R′″ is as previously defined,
   (b) a saturated or unsaturated, linear, branched or cyclic C₁-C₁₂ alkyl-R wherein R is as previously defined,
   (c) an aromatic ring which can be further substituted at any position by R wherein R is as previously defined, and
   (d) a saturated or unsaturated, linear, branched or cyclic C₁-C₁₂ alkyl optionally terminated by an aromatic ring which can be further substituted as defined in (c);
   with the proviso that when R₁ is O and R₂ and R₃ are OH, then R₄ is other than a straight or branched C₅-C₁₀ alkyl, C₅-C₁₀ alkenyl, C₅-C₈ cycloalkyl and C₅-C₈ cycloalkenyl;
   and pharmaceutically acceptable salts, esters or solvates thereof.

According to currently preferred embodiments, we now disclose compounds of the general formula (I) wherein R₁ is O, CH₂ or N—OH, R₂ and R₃ are each independently H, OH, OCH₃, succinate, fumarate or diethylphosphate, and R₄ is 1,1-dimethyl-pentyl, 1,1-dimethyl-heptyl, 1,1-dimethyl-pent-4-enyl, 1,1-dimethyl-hept-6-ynyl, 1,1-dimethyl-3-phenyl-propyl, 1,1-dimethyl-5-bromo-pentyl, 1,1-dimethyl-5-cyano-pentyl, 1,1,3-trimethyl-butyl, 1-methyl-1-p-chlorophenyl-ethyl, or 1-ethyl-1-methyl-propyl, with the proviso defined for formula (I).

According to another embodiment of the present invention, we disclose a compound of the general formula (II): 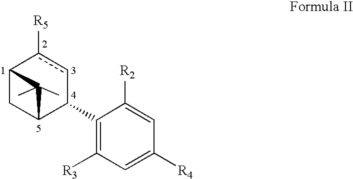
having a specific stereochemistry wherein C-5 is S, the protons at C-1 and C-5 are cis in relation to one another, the protons at C-4 and C-5 are trans, and C-2—C-3 is an optional double bond; and wherein:
R₅ is selected from the group consisting of
   (a) halogen or hydrogen,
   (b) —OR″, —N(R″)₂, —SR″, —S(O)(O)NR″, wherein at each occurrence R″ is independently selected from the group consisting of hydrogen, C(O)R′″, C(O)N(R′″)₂, C(S)R′″, saturated or unsaturated, linear, branched or cyclic C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂ alkyl,
   (c) a saturated or unsaturated, linear, branched or cyclic C₁-C₆ alkyl-SR″ or C₁-C₆ alkyl-S(O)(O)NR″, wherein R″ as previously defined,
   (d) —S(O)R^{b}, —S(O)(O)R^{b}, —S(O)(O)OR^{b} wherein R^{b} is selected from the group consisting of hydrogen, saturated or unsaturated, linear, branched or cyclic C₁-C₆ alkyl, C₁-C₆ alkyl-OR″, and C₁-C₆ alkyl-N(R″)₂, wherein at each occurrence R″ is as previously defined,
   (e) a saturated or unsaturated, linear, branched or cyclic C₁-C₆ alkyl-S(O)R^{b}, C₁-C₆ alkyl-S(O)(O)R^{b}, C₁-C₆ alkyl-S(O)(O)OR^{b} wherein R^{b} is as previously defined, and
   (f) —R^{c} wherein R^{c} is selected from the group consisting of saturated or unsaturated, linear, branched or cyclic C₁-C₆ alkyl, C₁-C₆ alkyl-OR″, C₁-C₆ alkyl-N(R″)₂, C₁-C₆ alkyl-C(O)OR″, and C₁-C₆ alkyl-C(O)N(R″)₂ wherein at each occurrence R″ is as previously defined;
R₂ and R₃ are each independently selected from the group consisting of
   (a) halogen,
   (b) —R″, —OR″, —N(R″)₂, —SR″, —S(O)(O)NR″, wherein at each occurrence R″ is as previously defined,
   (c) —S(O)R^{b}, —S(O)(O)R^{b}, —S(O)(O)OR^{b} wherein R^{b} is selected from the group consisting of hydrogen, saturated or unsaturated, linear, branched or cyclic C₁-C₆ alkyl, C₁-C₆ alkyl-OR″, and C₁-C₆ alkyl-N(R″)₂, wherein R″ is as previously defined, and
   (d) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear, branched or cyclic C₁-C₆ alkyl and R^{e} is at each occurrence selected from the group consisting of hydrogen and R^{d} as previously defined; and
R₄ is selected from the group consisting of
   (a) R wherein R is selected from the group consisting of hydrogen, halogen, OR′″, OC(O)R′″, C(O)OR′″, C(O)R′″, OC(O)OR′″, CN, NO₂, N(R′″)₂, NC(O)R′″, NC(O)OR′″, C(O)N(R′″)₂, NC(O)N(R′″)₂, SR′″, and C(S)R′″, wherein at each occurrence R′″ is as previously defined,
   (b) a saturated or unsaturated, linear, branched or cyclic C₁-C₁₂ alkyl-R wherein R is as previously defined,
   (c) an aromatic ring which can be further substituted at any position by R wherein R is as previously defined, and
   (d) a saturated or unsaturated, linear, branched or cyclic C₁-C₁₂ alkyl optionally terminated by an aromatic ring which can be further substituted as defined in (c);
   with the proviso that when R₅ is R^{c}, then R₄ is other than a straight or branched saturated C₁-C₁₂ alkyl chain, a straight or branched saturated —O—C₂—C₉ alkoxy chain optionally substituted at the terminal carbon by a phenyl group, and a straight or branched saturated C₁-C₇ alkyl chain terminated by a hydroxyl or by a straight or branched saturated —O—C₁-C₅ alkoxy chain;
   and pharmaceutically acceptable salts, esters or solvates thereof.

According to currently preferred embodiments, we now disclose compounds of the general formula (II) wherein R₅ is CH₂OC(O)C(CH₃)₃, OH or CH₃, R₂ and R₃ are each independently OH, H, or diethylphosphate, R₄ is CH₂OC(O)(CH₂)₃CH₃, 1,1-dimethyl-heptyl, 1,1-dimethyl-ethyl-phenyl, or 1,1-dimethyl-hept-6ynyl, and there is an optional double bond between C-2 and C-3, with the proviso defined for formula (II).

According to a further preferred embodiment of the present invention, we disclose a CB2 binding compound of the general formula (I): 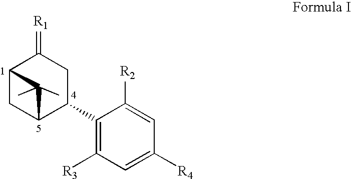
having a specific stereochemistry wherein C-5 is S, the protons at C-1 and C-5 are cis in relation to one another and the protons at C-4 and C-5 are trans; and wherein the substituents R₁-R₄ are as defined above.

According to an alternative preferred embodiment of the present invention, we disclose a CB2 binding compound of the general formula (II): 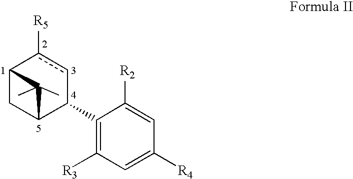
having a specific stereochemistry wherein C-5 is S, the protons at C-1 and C-5 are cis in relation to one another, the protons at C-4 and C-5 are trans, and C-2—C-3 is an optional double bond; and wherein the substituents R₂-R₅ are as defined for formula (II) with the proviso defined therein.

The present invention also encompasses a pharmaceutical composition comprising as an active ingredient a compound of general formula (III): 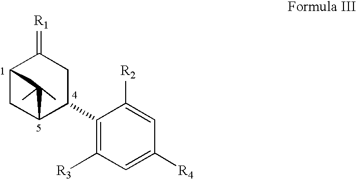
having a specific stereochemistry wherein C-5 is S, the protons at C-1 and C-5 are cis in relation to one another and the protons at C-4 and C-5 are trans; and wherein:
R₁ is selected from the group consisting of
   (a) O or S,
   (b) C(R′)₂ wherein R′ at each occurrence is independently selected from the group consisting of hydrogen, cyano, —OR″, —N(R″)₂, a saturated or unsaturated, linear, branched or cyclic C₁-C₆ alkyl, C₁-C₆ alkyl-OR″ or C₁-C₆ alkyl-N(R″)₂ wherein at each occurrence R″ is independently selected from the group consisting of hydrogen, C(O)R′″, C(O)N(R′″)₂, C(S)R′″, saturated or unsaturated, linear, branched or cyclic C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂ alkyl, and
   (c) NR″ or N—OR″ wherein R″ is as previously defined;
R₂ and R₃ are each independently selected from the group consisting of
   (a) halogen,
   (b) —R″, —OR″, —N(R″)₂, —SR″, —S(O)(O)NR″, wherein at each occurrence R″ is as previously defined,
   (c) —S(O)R^{b}, —S(O)(O)R^{b}, —S(O)(O)OR^{b} wherein R^{b} is selected from the group consisting of hydrogen, saturated or unsaturated, linear, branched or cyclic C₁-C₆ alkyl, C₁-C₆ alkyl-OR″, and C₁-C₆ alkyl-N(R″)₂, wherein R″ is as previously defined, and
   (d) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e}₂, wherein R^{d} is a saturated or unsaturated, linear, branched or cyclic C₁-C₆ alkyl and R^{e} is at each occurrence selected from the group consisting of hydrogen and R^{d} as previously defined; and
R₄ is selected from the group consisting of
   (a) R wherein R is selected from the group consisting of hydrogen, halogen, OR′″, OC(O)R′″, C(O)OR′″, C(O)R′″, OC(O)OR′″, CN, NO₂, N(R′″)₂, NC(O)R′″, NC(O)OR′″, C(O)N(R′″)₂, NC(O)N(R′″)₂, SR′″, and C(S)R′″, wherein at each occurrence R′″ is as previously defined,
   (b) a saturated or unsaturated, linear, branched or cyclic C₁-C₁₂ alkyl-R wherein R is as previously defined,
   (c) an aromatic ring which can be further substituted at any position by R wherein R is as previously defined, and
   (d) a saturated or unsaturated, linear, branched or cyclic C₁-C₁₂ alkyl optionally terminated by an aromatic ring which can be further substituted as defined in (c);
   and pharmaceutically acceptable salts, esters or solvates thereof.

According to currently preferred embodiments, we now disclose pharmaceutical compositions comprising as an active ingredient a compound of general formula (III) wherein R₁ is O, CH₂ or N—OH, R₂ and R₃ are each independently H, OH, OCH₃, succinate, fumarate or diethylphosphate, and R₄ is 1,1-dimethyl-pentyl, 1,1-dimethyl-heptyl, 1,1-dimethyl-pent-4-enyl, 1,1-dimethyl-hept-6-ynyl, 1,1-dimethyl-3-phenyl-propyl, 1,1-dimethyl-5-bromo-pentyl, 1,1-dimethyl-5-cyano-pentyl, 1,1,3-trimethyl-butyl, 1-methyl-1-p-chlorophenyl-ethyl, or 1-ethyl-1-methyl-propyl.

The present invention further encompasses a pharmaceutical composition comprising as an active ingredient a compound of general formula (II): 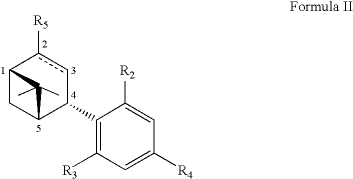
having a specific stereochemistry wherein C-5 is S, the protons at C-1 and C-5 are cis in relation to one another, the protons at C-4 and C-5 are trans, and C-2—C-3 is an optional double bond; and wherein:
R₅ is selected from the group consisting of
   (a) halogen or hydrogen,
   (b) —R″, —N(R″)₂, —SR″, —S(O)(O)NR″, wherein at each occurrence R″ is independently selected from the group consisting of hydrogen, C(O)R′″, C(O)N(R′″)₂, C(S)R′″, saturated or unsaturated, linear, branched or cyclic C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂ alkyl,
   (c) a saturated or unsaturated, linear, branched or cyclic C₁-C₆ alkyl-SR″ or C₁-C₆ alkyl-S(O)(O)NR″, wherein R″ as previously defined,
   (d) —S(O)R^{b}, —S(O)(O)R^{b}, —S(O)(O)OR^{b} wherein R^{b} is selected from the group consisting of hydrogen, saturated or unsaturated, linear, branched or cyclic C₁-C₆ alkyl, C₁-C₆ alkyl-OR″, and C₁-C₆ alkyl-N(R″)₂, wherein at each occurrence R″ is as previously defined,
   (e) a saturated or unsaturated, linear, branched or cyclic C₁-C₆ alkyl-S(O)R^{b}, C₁-C₆ alkyl-S(O)(O)R^{b}, C₁-C₆ alkyl-S(O)(O)OR^{b} wherein R^{b} is as previously defined, and
   (f) —R^{c} wherein R^{c} is selected from the group consisting of saturated or unsaturated, linear, branched or cyclic C₁-C₆ alkyl, C₁-C₆ alkyl-OR″, C₁-C₆ alkyl-N(R″)₂, C₁-C₆ alkyl-C(O)OR″, and C₁-C₆ alkyl-C(O)N(R″)₂ wherein at each occurrence R″ is as previously defined;
R₂ and R₃ are each independently selected from the group consisting of
   (a) halogen,
   (b) —R″, —OR″, —N(R″)₂, —SR″, —S(O)(O)NR″, wherein at each occurrence R″ is as previously defined,
   (c) —S(O)R^{b}, —S(O)(O)R^{b}, —S(O)(O)OR^{b} wherein R^{b} is selected from the group consisting of hydrogen, saturated or unsaturated, linear, branched or cyclic C₁-C₆ alkyl, C₁-C₆ alkyl-OR″, and C₁-C₆ alkyl-N(R″)₂, wherein R″ is as previously defined, and
   (d) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear, branched or cyclic C₁-C₆ alkyl and R^{e} is at each occurrence selected from the group consisting of hydrogen and R^{d} as previously defined; and
R₄ is selected from the group consisting of
   (a) R wherein R is selected from the group consisting of hydrogen, halogen, OR′″, OC(O)R′″, C(O)OR′″, C(O)R′″, OC(O)OR′″, CN, NO₂, N(R′″)₂, NC(O)R′″, NC(O)OR′″, C(O)N(R′″)₂, NC(O)N(R′″)₂, SR′″, and C(S)R′″, wherein at each occurrence R′″ is as previously defined,
   (b) a saturated or unsaturated, linear, branched or cyclic C₁-C₁₂ alkyl-R wherein R is as previously defined,
   (c) an aromatic ring which can be further substituted at any position by R wherein R is as previously defined, and
   (d) a saturated or unsaturated, linear, branched or cyclic C₁-C₁₂ alkyl optionally terminated by an aromatic ring which can be further substituted as defined in (c);
   with the proviso that. when R₅ is R^{c}, then R₄ is other than a straight or branched saturated C₁-C₁₂ allyl chain, a straight or branched saturated —O—C₂-C₉ alkoxy chain optionally substituted at the terminal carbon by a phenyl group, and a straight or branched saturated C₁-C₇ alkyl chain terminated by a hydroxyl or by a straight or branched saturated —O—C₁-C₅ alkoxy chain;
   and pharmaceutically acceptable salts, esters or solvates thereof.

According to currently preferred embodiments, we now disclose pharmaceutical compositions comprising as an active ingredient a compound of general formula (II) wherein R₅ is CH₂OC(O)C(CH₃)₃, OH or CH₃, R₂ and R₃ are each independently OH, H, or diethylphosphate, R₄ is CH₂OC(O)(CH₂)₃CH₃, 1,1-dimethyl-heptyl, 1,1-dimethyl-ethyl-phenyl, or 1,1-dimethyl-hept-6-ynyl, and there is an optional double bond between C-2 and C-3, with the proviso defined for formula (II).

The novel compositions may contain in addition to the active ingredient conventional pharmaceutically acceptable carriers, diluents and excipients necessary to produce a physiologically acceptable and stable formulation.

The pharmaceutical compositions can be administered by any conventional and appropriate route including oral, parenteral, intravenous, intramuscular, intralesional, subcutaneous, transdermal, intrathecal, rectal or intranasal.

A further aspect of the present invention provides a method of treating a patient by stimulating CB2 receptors, which comprises administering to said patient a pharmaceutical composition comprising a therapeutically effective amount of a compound of the general formulae (II) and (III) according to the present invention.

Accordingly, the present invention provides a method comprising administering to an individual in need thereof of a therapeutically effective amount of a compound of the general formulae (II) and (III) for immunomodulation and for indications amenable to CB2 receptor modulation. The indications include but are not limited to: autoimmune diseases including rheumatoid arthritis, multiple sclerosis, systemic lupus erythematosis, myasthenia gravis, diabetes mellitus type I hepatitis and psoriasis and immune related disorders including but not limited to tissue rejection in organ transplants, malabsorption syndromes such as celiac disease, pulmonary diseases such as asthma and Sjögren's syndrome, inflammation including inflammatory bowel disease, pain including peripheral, visceral, neuropathic, inflammatory and referred pain, muscle spasticity, cardiovascular disorders including arrhythmia, hypertension and myocardial ischemia, neurological disorders including stroke, migraine and cluster headaches, neurodegenerative diseases including Parkinson's disease, Alzheimer's disease, amyotrophic lateral sclerosis, Huntington's chorea, prion-associated neurodegeneration, CNS poisoning and certain types of cancer.

The present invention encompasses the use of the compounds of the general formulae (II) and III for the preparation of a medicament for the treatment and prevention autoimmune diseases including but not limited to rheumatoid arthritis, multiple sclerosis, systemic lupus erythematosis, myasthenia gravis, diabetes mellitus type I hepatitis, psoriasis and immune related disorders including but not limited to tissue rejection in organ transplants, malabsorption syndromes such as celiac disease, pulmonary diseases such as asthma and Sjögren's syndrome, inflammation including inflammatory bowel disease, pain including peripheral, visceral, neuropathic, inflammatory and referred pain, muscle spasticity, cardiovascular disorders including arrhythmia, hypertension and myocardial ischemia, neurological disorders including stroke, migraine and cluster headaches, neurodegenerative diseases including Parkinson's disease, Alzheimer's disease, amyotrophic lateral sclerosis, Huntington's chorea, prion-associated neurodegeneration, CNS poisoning and certain types of cancer, as shown in the specification.

While the compounds and compositions of the present invention are specifically designed to serve as ligands of the peripheral cannabinoid receptor CB2, they may also possess other desirable therapeutic attributes of the class of compounds referred to as “non-classical cannabinoids” whether or not mediated via the CB2 receptor. Thus the compounds and compositions of general formulae (I) to (III) have neuroprotective properties in addition to their immunomodulatory activity.

As exemplified herein below, we have now found that the known CB2 specific agonist HU-308, the full chemical name of which is (+) {4-[4-(1,1-dimethylheptyl)-2,6-dimethoxy-phenyl]-6,6-dimethyl-bicyclo[3.1.1]hept-2-en-2-yl}-methanol, also disclosed in WO 01/32169 as (+) 4-[2,6-dimethoxy-4-(1,1-dimethylheptyl)phenyl]-6,6-dimethyl-bicyclo[3.1.1]hept-2-ene-2-carbinol, is not only effective in the treatment of peripheral pain but also in the treatment of neuropathic pain. Moreover, we have now found that HU-308 is particularly effective in the treatment and the prevention of Parkinson's disease.

### BRIEF DESCRIPTION OF THE FIGURES

To assist in the understanding of the invention and in particular of the data that are given in the Examples, the following drawing figures are presented herein:

FIG. 1 shows the binding of selected bicyclic compounds to the CB1 and CB2 human cannabinoid receptors.

FIG. 2 shows the effect of selected bicyclic compounds on secretion from activated macrophages. Panel A displays effect on IL-1β secretion at single dose. Panel B displays effect on PGE₂ secretion at various doses.

FIG. 3 shows the effect of compounds of the present invention at various doses on IL-2 secretion from activated T cells.

FIG. 4 shows the effect of a compound of the present invention, at various doses, in the EAE model for multiple sclerosis.

FIG. 5 shows the effect of the known CB2 agonist HU-308, and compounds of the present invention, at various doses, in the DTH model for allergic or other immune reactions.

FIG. 6 shows the effect of the known CB2 agonist HU-308 in the MPTP model for Parkinson's disease.

FIG. 7 shows the effect of a compound of the present invention at two doses in the Constriction Nerve Injury model for Chronic Neuropathic pain.

FIG. 8 shows the effect of compounds of the present invention, at various doses, in the Tail Flick model for Acute Peripheral pain. Panel A presents the results obtained 30 minutes after treatment and panel B the results obtained 90 minutes after treatment.

FIG. 9 shows the effect of a single dose of compounds of the present invention, as compared to vehicle and morphine over 5.5 hours in the Tail Flick model for Acute Peripheral pain. Panel A presents the results as latency time while panel B presents the results as percent animals in the treated group displaying latency times twice higher than vehicle treated animals.

FIG. 10 shows the effect of a compound of the present invention, as compared to morphine, on the development of tolerance as measured in the Tail Flick model. Panel A shows results in latency time and panel B shows results in percent of animals showing analgesia

### DETAILED DESCRIPTION OF THE INVENTION

The present invention provides novel compounds belonging to the non-classical cannabinoids, as well as pharmaceutical compositions comprising these compounds, and methods of using such compounds and compositions. The compounds of this class show affinity for cannabinoid receptors. The preferred novel compounds of this invention show affinity for the peripheral human cannabinoid receptor, CB2. The compositions of the present invention have been shown to possess immunomodulatory, anti-inflammatory, analgesic, neuroprotective and certain anti-tumoral properties. The action of some compounds may result in modulation of transcription of genes involved in immunomodulation and inflammation or of the signal transduction components involved in such processes.

Cannabinoids are believed to exert their physiological effects mainly through receptor mediated mechanisms, but non-receptor mediated activities have been reported (Felder C. C. et al., Mol. Pharmacol. 42: 838-45, 1992). Moreover, there is growing pharmacological evidence for the existence of additional types of cannabinoid receptors in addition to CB1 and CB2 discovered so far (Howlett A. C. et al., Pharmacol. Review 54: 161-202, 2002). Thus, though the most probable mechanism of action of compounds of the invention is through their selective binding to the CB2 receptor and functional coupling to specific signal transduction pathways, we cannot rule out alternative mechanisms, for instance either through binding to additional yet unidentified cannabinoid receptors or through non-receptor mediated means, or a combination of such mechanisms.

In the present specification the term “prodrug” represents compounds which are rapidly transformed in vivo to the parent compounds of formulae (I) to (III), for example by hydrolysis in blood. Some of the compounds of formulae (I) to (III) are capable of further forming pharmaceutically acceptable salts and esters. “Pharmaceutically acceptable salts and esters” means any salt and ester that is pharmaceutically acceptable and has the desired pharmacological properties. Such salts include salts that may be derived from an inorganic or organic acid, or an inorganic or organic base, including amino acids, which is not toxic or undesirable in any way. The present invention also includes within its scope solvates of compounds of formulae (I) to (III) and salts thereof, for example, hydrates. All of these pharmaceutical forms are intended to be included within the scope of the present invention.

In the present specification and claims which follow “prophylactically effective” is intended to qualify the amount of compound which will achieve the goal of prevention, reduction or eradication of the risk of occurrence of the disorder, while avoiding adverse side effects. The term “therapeutically effective” is intended to qualify the amount of compound that will achieve, with no adverse effects, alleviation, diminished progression or treatment of the disorder, once the disorder cannot be further delayed and the patients are no longer asymptomatic. The compositions of the present invention are prophylactic as well as therapeutic.

The “individual” or “patient” for purposes of treatment includes any human or mammalian subject affected by any of the diseases where the treatment has beneficial therapeutic impact.

By virtue of their anti-inflammatory and immunomodulatory properties, it will be recognized that the compositions according to the present invention will be useful for treating indications having an inflammatory or autoimmune mechanism involved in their etiology or pathogenesis exemplified by arthritis, including rheumatoid arthritis, arthritis, multiple sclerosis, systemic lupus erythematosus (SLE), myasthenia gravis, diabetes mellitus type I, hepatitis and psoriasis, immune related disorders including but not limited to tissue rejection in organ transplants, malabsorption syndromes such as celiac, pulmonary diseases such as asthma and Sjögren's syndrome, inflammatory bowel disease, and rheumatic diseases.

While the compounds and compositions of the present invention were designed to be CB2 ligands they share other properties of this class of non-classical cannabinoids including neuroprotective properties (U.S. Pat. No. 5,434,295). By virtue of their neuroprotective properties, it will be recognized that the compositions according to the present invention will be useful in treating neurological disorders including but not limited to stroke, migraine, and cluster headaches. The composition of the present invention may also be effective in treating certain chronic degenerative diseases that are characterized by gradual selective neuronal loss. In this connection, the compositions of the present invention are contemplated as therapeutically effective in the treatment of Parkinson's disease, Alzheimer's disease, amyotrophic lateral sclerosis, Huntington's chorea and prion-associated neurodegeneration. Neuroprotection conferred by CB2 agonists could also be effective in protection and/or treatment of neurotoxic agents, such as nerve gas, as well as other insults to brain or nervous tissue by way of chemical or biological agents.

By virtue of their analgesic properties it will be recognized that the compositions according to the present invention will be useful in treating pain including peripheral, visceral, neuropathic, inflammatory and referred pain. The compositions of the present invention may also be effective in cardioprotection from arrhythmia, hypertension and myocardial ischemia. The compositions of the present invention may also be effective in the treatment of muscle spasm and tremor.

Another feature of the present invention is the ability of the disclosed compounds to prevent or treat certain cancers, including malignant brain tumors, skin tumors, lung adenocarcinoma, glioma, thyroid epithelioma, where CB2 binding ligands may trigger apoptosis of tumor cells as well as inhibiting tumor angiogenesis.

Moreover, we have found that some preferred CB2 binding compounds are may also act by modulating the transcription of genes involved in immunomodulation and inflammation.

Furthermore, we now disclose that the known CB2 specific agonist HU-308 that was found to be effective in the treatment of peripheral pain is unexpectedly also effective in the treatment of neuropathic pain as assessed by chronic constriction of the sciatic nerve in rodent models.

Additionally, it was also discovered that HU-308 reduced significantly the extent of cell death produced in the Substantia Nigra of mice treated with the neurotoxin MPTP. This suggests that this compound may prove especially effective in the treatment of Parkinson's disease.

Bicyclic compounds shown to have high affinity and specificity for the CB2 receptor have been disclosed by Makriyannis and co-worker in international patent application WO 01/28497. A person skilled in the art would discern in that disclosure that the compounds disclosed are of opposite stereochemistry to those of the present invention, since the dimethyl of the four member ring is below the plane of the terpenic ring while the aryl group lies above this same plane, as drawn in formulae I and II of that disclosure. According to the nomenclature adopted in the present disclosure, the Makriyannis' compounds are of stereochemical orientation wherein C-1, C-4 and C-5 are R.

In general, it has been possible to functionally differentiate between the R and S enantiomers of cannabinoid and cannabinoid-related compounds. The compounds HU-210 and HU-211 exemplify this. HU-210 is the (−)(3R,4R) enantiomer of the synthetic cannabinoid, 7-hydroxy-Δ⁶-tetrahydrocannabinol-1,1-dimethyl-heptyl. HU-211 is the (+)(3S,4S) enantiomer of that compound. In contrast to HU-210, HU-211 exhibits low affinity to the cannabinoid receptors and is thus non-psychotropic. In addition, it functions as a noncompetitive NMDA-receptor antagonist and as a neuroprotective agent, two properties absent in HU-210 (U.S. Pat. No. 5,284,867).

The inventors of the present invention have unexpectedly found that the enantiomers of stereochemistry opposite to the compounds disclosed in WO 01/28497 are effective CB2 receptor ligands. The present disclosure teaches novel derivatives of (+) α-pinene wherein C-5 is S, the protons at C-1 and C-5 are cis in relation to one another and the protons at C-4 and C-5 are trans, as depicted in formulae (I) to (III). Preferred compounds of the present invention not only have a higher affinity toward CB2 and a better selectivity as compared to their known enantiomers, but are also more efficient in vivo as established by experimental results. In this context it should be noted that the enantiomers disclosed in WO 01/28497 were tested by their inventors only for binding, and for no additional biological activity neither in vitro nor in vivo.

Moreover, the present invention relates to the use of these novel CB2 ligands for the preparation of compounds to prevent or treat autoimmune diseases and related disorders, inflammation, pain, muscle spasticity, cardiovascular disorders, neurological disorders, neurodegenerative diseases, CNS poisoning and certain types of cancer.

In the present invention we will refer to the following numbering of positions in the ring structure, where positions 1, 4 and 5 are chiral centers. The stereochemistry of the compounds disclosed in the present invention is such that C-5 is S, the protons at C-1 and C-5 are cis in relation to one another and the protons at C-4 and C-5 are trans as shown in formula (IV):

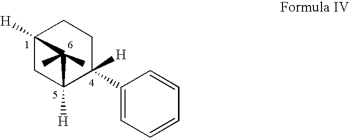

In the present invention, binding affinity is represented by the IC₅₀ value, namely the concentration of a test compound that will displace 50% of a radiolabeled agonist from the CB receptors. Preferred compounds display IC₅₀ value for CB2 binding of 50 nM or lower, preferably of 30 nM or lower, more preferably of 10 nM or lower and most preferably of 1 nM or lower. “CB2 specific or selective” denotes compounds with a ratio of CB2/CB1 binding affinity that is at least 10, preferably 20, more preferably 50 and most preferably 100 or greater. Preferably these ratios will be obtained for human CB1 and CB2 receptors. The selectivity toward CB2, denoted CB2/CB1 affinity, is calculated as the IC₅₀ value obtained by the test compound for the displacement of the CB1 specific radioligand divided by the IC₅₀ value obtained for the displacement of the CB2 specific radioligand, i.e. the IC₅₀ CB1/IC₅₀ CB2. Some of the preferred compounds of the present invention do not necessarily share both properties, in other words some have an IC₅₀ for CB2 of 1 nM or lower but a ratio of only about 30.

Throughout this specification, certain compounds of the present invention may be referred to by capital letters rather than by their full chemical names. The alkyl substituents can be saturated or unsaturated, linear, branched or cyclic, the latter only when the number of carbon atoms in the alkyl chain is greater than or equal to three. OC(O)R represents esters, OC(O)NR carbamates, OC(S)R thioesters, NR₂ amines, NRC(O)R amides, NRC(O)NR ureas, NRC(S)R thioamides, SR thiols or sulfides, S(O)R sulfoxides, SC(O)R thioesters, SC(O)NR thiocarbamates, SC(S)R dithioesters, S(O)(O)R sulfones, S(O)(O)OR sulfonates, S(O)(O)NR sulfonamides, S(O)(O)NC(O)R acylsulfonamides, S(O)(O)NC(O)NR sulfonurea, S(O)(O)NC(S)R thioacylsulfonamide when R is a hydrogen or an alkyl chain.

The present invention relates to compounds of the general formula (I): 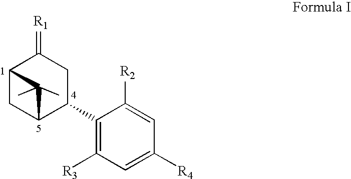
having a specific stereochemistry wherein C-5 is S, the protons at C-1 and C-5 are cis in relation to one another and the protons at C-4 and C-5 are trans; and wherein the substituents R₁-R₄ are as defined for formula (I) with the proviso defined therein.

According to currently preferred embodiments, we now disclose compounds of the general formula (I) wherein R₁ is O, CH₂ or N—OH, R₂ and R₃ are each independently H, OH, OCH₃, succinate, fumarate or diethylphosphate, and R₄ is 1,1-dimethyl-pentyl, 1,1-dimethyl-heptyl, 1,1-dimethyl-pent-4-enyl, 1,1-dimethyl-hept-6-ynyl, 1,1-dimethyl-3-phenyl-propyl, 1,1-dimethyl-5-bromo-pentyl, 1,1-dimethyl-5-cyano-pentyl, 1,1,3-trimethyl-butyl, 1-methyl-1-p-chlorophenyl-ethyl, or 1-ethyl-1-methyl-propyl, with the proviso as defined for formula (I).

According to other currently preferred embodiments, we now disclose compounds of the general formula (I) wherein: R₁ is O, R₂ and R₃ are OCH₃, and R₄ is 1,1-dimethyl-heptyl; R₁ is N—OH, R₂ and R₃ are OH, and R₄ is 1,1-dimethyl-heptyl; R₁ is O, R₂ and R₃ are OH, and R₄ is 1,1-dimethyl-hept-6-ynyl; R₁ is O, R₂ and R₃ are OH, and R₄ is 1,1-dimethyl-3-phenyl-propyl; R₁ is O, R₂ and R₃ are OH, and R₄ is 1-methyl-1-p-chlorophenyl-ethyl; R₁ is O, R₂ and R₃ are OH, and R₄ is 1,1-dimethyl-5-bromo-pentyl; R₁ is O, R₂ and R₃ are OH, and R₄ is 1,1-dimethyl-5-cyano-pentyl; R₁ is O, R₂ is succinate, R₃ is OH, and R₄ is 1,1-dimethyl-heptyl; R₁ is O, R₂ and R₃ are succinate, and R₄ is 1,1-dimethyl-heptyl; R₁ is O, R₂ is succinate, R₃ is OH, and R₄ is 1,1-dimethyl-pentyl; R₁ is O, R₂ is OH, R₃ is OCH₃, and R₄ is 1,1-dimethyl-heptyl; R₁ is O, R₂ and R₃ are H, and R₄ is 1,1-dimethyl-heptyl; R₁ is CH₂, R₂ and R₃ are OCH₃, and R₄ is 1,1-dimethyl-heptyl; R₁ is O, R₂ and R₃ are diethylphosphate, and R₄ is 1,1-dimethyl-heptyl; R₁ is O, R₂ is diethylphosphate and R₃ is OH, and R₄ is 1,1-dimethyl-heptyl; R₁ is CH₂, R₂ and R₃ are diethylphosphate, and R₄ is 1,1-dimethyl-heptyl; and R₁ is O, R₂ is fumarate, R₃ is OH, and R₄ is 1,1-dimethyl-heptyl.

The present invention also relates to compounds of the general formula (II): 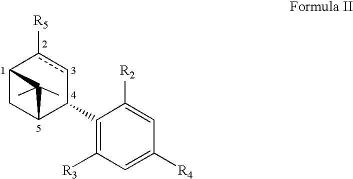
having a specific stereochemistry wherein C-5 is S, the protons at C-1 and C-5 are cis in relation to one another, the protons at C-4 and C-5 are trans, and C-2—C-3 is an optional double bond; and wherein the substituents R₂-R₅ are as defined for formula (II) with the proviso defined therein.

According to currently preferred embodiments, we now disclose compounds of the general formula (II) wherein R₅ is CH₂OC(O)C(CH₃)₃, OH or CH₃, R₂ and R₃ are each independently OH, H, or diethylphosphate, R⁴ is CH₂OC(O)CH₂)₃CH₃, 1,1-dimethyl-heptyl, 1,1-dimethyl-ethyl-phenyl, or 1,1-dimethyl-hept-6-ynyl, and there is an optional double bond between C-2 and C-3, with the proviso defined for formula (II).

According to other currently preferred embodiments, we now disclose compounds of the general formula (II) wherein: R₅ is OH, R₂ and R₃ are OH, R₄ is 1,1-dimethyl-heptyl and there is a single bond between C-2 and C-3; R₅ is CH₃, R₂ and R₃ are OH, R₄ is 1,1-dimethyl-hept-6-ynyl and there is a double bond between C-2 and C-3; R₅ is CH₃, R₂ and R₃ are OH, R₄ is 1,1-dimethyl-ethyl-phenyl and there is a double bond between C-2 and C-3; R₅ is OH, R₂ and R₃ are H, R₄ is 1,1-dimethyl-heptyl and there is a single bond between C-2 and C-3; R₅ is CH₃, R₂ and R₃ are OH, R₄ is CH₂OC(O)(CH₂)₃CH₃ and there is a double bond between C-2 and C-3; and R₅ is OH, R₂ and R₃ are diethylphosphate, R₄ is 1,1-dimethyl-heptyl and there is a single bond between C-2 and C-3.

The present invention further relates to CB2 binding compounds of the general formula (I): 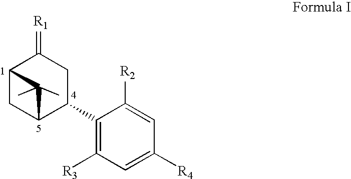
having a specific stereochemistry wherein C-5 is S, the protons at C-1 and C-5 are cis in relation to one another and the protons at C-4 and C-5 are trans; and wherein the substituents R₁-R₄ are as defined for formula (I).

The present invention further relates to CB2 binding compounds of the general formula (II): 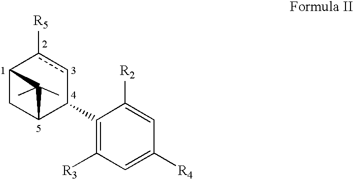
having a specific stereochemistry wherein C-5 is S, the protons at C-1 and C-5 are cis in relation to one another, the protons at C-4 and C-5 are trans, and C-2—C-3 is an optional double bond; and wherein the substituents R₂-R₅ are as defined for formula (II) with the proviso defined therein.

The present invention relates to pharmaceutical compositions for the purposes set out above, comprising as an active ingredient a compound of the general formula (III): 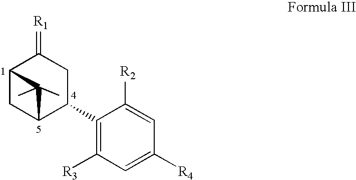
having a specific stereochemistry wherein C-5 is S, the protons at C-1 and C-5 are cis in relation to one another and the protons at C-4 and C-5 are trans; and wherein the substituents R₁-R₄ are as defined for formula (III).

According to currently preferred embodiments, we now disclose pharmaceutical compositions comprising as an active ingredient a compound of general formula (III) wherein R₁ is O, CH₂ or N—OH, R₂ and R₃ are each independently H, OH, OCH₃, succinate, fumarate or diethylphosphate, and R₄ is 1,1-dimethyl-pentyl, 1,1-dimethyl-heptyl, 1,1-dimethyl-pent 4 enyl, 1,1-dimethyl-hept-6-ynyl, 1,1-dimethyl-3-phenyl-propyl, 1,1-dimethyl-5-bromo-pentyl, 1,1-dimethyl-5-cyano-pentyl, 1,1,3-trimethyl-butyl, 1-methyl-1-p-chlorophenyl-ethyl, or 1-ethyl-1-methyl-propyl.

According to other currently preferred embodiments, we now disclose pharmaceutical compositions comprising as an active ingredient a compound of general formula (III) wherein: R₁ is O, R₂ and R₃ are OH, and R₄ is 1,1-dimethyl-heptyl; R₁ is O, R₂ and R₃ are OCH₃, and R₄ is 1,1-dimethyl-heptyl; R₁ is N—OH, R₂ and R₃ are OH, and R₄ is 1,1-dimethyl-heptyl; R₁ is O, R₂ and R₃ are OH, and R₄ is 1,1-dimethyl-hept-6-ynyl; R₁ is O, R₂ and R₃ are OH, and R₄ is 1,1-dimethyl-3-phenyl-propyl; R₁ is O, R₂ and R₃ are OH, and R₄ is 1,1,3-trimethyl-butyl; R₁ is O, R₂ and R₃ are OH, and R₄ is 1-methyl-1-p-chlorophenyl-ethyl; R₁ is O, R₂ and R₃ are OH, and R₄ is 1,1-dimethyl-pentyl; R₁ is O, R₂ and R₃ are OH, and R₄ is 1-ethyl-1-methyl-propyl; R₁ is O, R₂ and R₃ are OH, and R₄ is 1,1-dimethyl-5-bromo-pentyl; R₁ is O, R₂ and R₃ are OH, and R₄ is 1,1-dimethyl-5-cyano-pentyl; R₁ is O, R₂ is succinate, R₃ is OH, and R₄ is 1,1-dimethyl-heptyl; R₁ is O, R₂ and R₃ are succinate, and R₄ is 1,1-dimethyl-heptyl; R₁ is O, R₂ is succinate, R₃ is OH, and R₄ is 1,1-dimethyl-pentyl; R₁ is O, R₂ is OH, R₃ is OCH₃, and R₄ is 1,1-dimethyl-heptyl; R₁ is O, R₂ and R₃ are H, and R₄ is 1,1-dimethyl-heptyl; R₁ is CH₂, R₂ and R₃ are OCH₃, and R₄ is 1,1-dimethyl-heptyl; R₁ is O, R₂ and R₃ are diethylphosphate, and R₄ is 1,1-dimethyl-heptyl; R₁ is O, R₂ is diethylphosphate and R₃ is OH, and R₄ is 1,1-dimethyl-heptyl; R₁ is O, R₂ and R₃ are OH, and R₄ is 1,1 diethyl-pent-4-enyl; R₁ is CH₂, R₂ and R₃ are diethylphosphate, and R₄ is 1,1-dimethyl-heptyl; and R₁ is O, R₂ is fumarate, R₃ is OH, and R₄ is 1,1-dimethyl-heptyl.

The present invention further relates to pharmaceutical compositions for the purposes set out above comprising as an active ingredient a compound of the general formula (II): 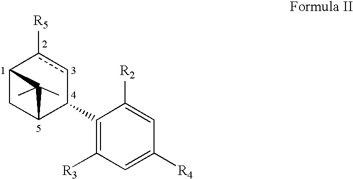
having a specific stereochemistry wherein C-5 is S, the protons at C-1 and C-5 are cis in relation to one another, the protons at C-4 and C-5 are trans and C-2—C-3 is an optional double bond; and wherein the substituents R₂-R₅ are as defined for formula (II) with the proviso defined therein.

According to a currently preferred embodiment, we disclose a pharmaceutical composition comprising as an active ingredient a compound of the general formula (II) wherein R₅ is CH₂OC(O)C(CH₃)₃, OH or CH₃, R₂ and R₃ are each independently OH, H, or diethylphosphate, R₄ is CH₂OC(O)(CH₂)₃CH₃, 1,1-dimethyl-heptyl, 1,1-dimethyl-ethyl-phenyl, or 1,1-dimethyl-hept-6-ynyl, and there is an optional double bond between C-2 and C-3, with the proviso defined for formula (II).

According to other currently preferred embodiments, we now disclose pharmaceutical compositions comprising as an active ingredient a compound of general formula (II) wherein: R₅ is OH, R₂ and R₃ are OH, R₄ is 1,1-dimethyl-heptyl and there is a single bond between C-2 and C-3; R₅ is CH₃, R₂ and R₃ are OH, R₄ is 1,1-dimethyl-hept-6-ynyl and there is a double bond between C-2 and C-3; R₅ is CH₃, R₂ and R₃ are OH, R₄ is 1,1-dimethyl-ethyl-phenyl and there is a double bond between C-2 and C-3; R₅ is OH, R₂ and R₃ are H, R₄ is 1,1-dimethyl-heptyl and there is a single bond between C-2 and C-3; R₅ is CH₃, R₂ and R₃ are OH, R₄ is CH₂OC(O)(CH₂)₃CH₃ and there is a double bond between C-2 and C-3; and R₅ is OH, R₂ and R₃ are diethylphosphate, R₄ is 1,1-dimethyl-heptyl and there is a single bond between C-2 and C-3.

The novel non-classical cannabinoids according to the present invention most preferably bind efficiently to the CB2 receptor but weakly to CB1 receptor, the latter known to mediate the psychotropic activity in the CNS in addition to the beneficial therapeutic effects.

The present invention further relates to new therapies utilizing the compositions of the present invention for the prevention and treatment of autoimmune diseases including rheumatoid arthritis, multiple sclerosis, systemic lupus erythematosis, myasthenia gravis, diabetes mellitus type I, hepatitis and psoriasis and immune related disorders including but not limited to tissue rejection in organ transplants, malabsorption syndromes such as celiac disease, pulmonary diseases such as asthma and Sjögren's syndrome, inflammation including inflammatory bowel disease, pain including peripheral, visceral, neuropathic, inflammatory and referred pain, muscle spasticity, cardiovascular disorders including arrhythmia, hypertension and myocardial ischemia, neurological disorders including stroke, migraine and cluster headaches, neurodegenerative diseases including Parkinson's disease, Alzheimer's disease, amyotrophic lateral sclerosis, Huntington's chorea, prion-associated neurodegeneration, CNS poisoning and certain types of cancer.

The novel compositions contain, in addition to the active ingredient, conventional pharmaceutically acceptable carriers, diluents and excipients necessary to produce a physiologically acceptable and stable formulation. For compounds having solubility problems, and some compounds of the present invention are characteristically hydrophobic and practically insoluble in water with high lipophilicity, as expressed by their high octanol/water partition coefficient and log P values, formulation strategies to prepare acceptable dosage forms will be applied. Enabling therapeutically effective and convenient administration of the compounds of the present invention is an integral part of this invention.

For water soluble compounds standard formulations will be utilized. Solid compositions for oral administration such as tablets, pills, capsules, softgels or the like may be prepared by mixing the active ingredient with conventional, pharmaceutically acceptable ingredients such as corn starch, lactose, sucrose, mannitol, sorbitol, talc, polyvinylpyrrolidone, polyethyleneglycol, cyclodextrins, dextrans, glycerol, polyglycolized glycerides, tocopheryl polyethyleneglycol succinate, sodium lauryl sulfate, polyethoxylated castor oils, non-ionic surfactants, stearic acid, magnesium stearate, dicalcium phosphate and gums as pharmaceutically acceptable diluents. The tablets or pills can be coated or otherwise compounded with pharmaceutically acceptable materials known in the art, such as microcrystalline cellulose and cellulose derivatives such as hydroxypropylmethylcellulose (HPMC), to provide a dosage form affording prolonged action or sustained release. Other solid compositions can be prepared as suppositories, for rectal administration. Liquid forms may be prepared for oral administration or for injection, the term including but not limited to subcutaneous, transdermal, intravenous, intrathecal, intralesional, adjacent to or into tumors, and other parenteral routes of administration. The liquid compositions include aqueous solutions, with or without organic cosolvents, aqueous or oil suspensions including but not limited to cyclodextrins as suspending agent, flavored emulsions with edible oils, triglycerides and phospholipids, as well as elixirs and similar pharmaceutical vehicles. In addition, the compositions of the present invention may be formed as aerosols, for intranasal and like administration. Topical pharmaceutical compositions of the present invention may be formulated as solution, lotion, gel, cream, ointment, emulsion or adhesive film with pharmaceutically acceptable excipients including but not limited to propylene glycol, phospholipids, monoglycerides, diglycerides, triglycerides, polysorbates, surfactants, hydrogels, petrolatum or other such excipients as are known in the art.

Prior to their use as medicaments, the pharmaceutical compositions will generally be formulated in unit dosage. The active dose for humans is generally in the range of from 0.05 mg to about 50 mg per kg body weight, in a regimen of 1-4 times a day. The preferred range of dosage is from 0.1 mg to about 20 mg per kg body weight. However, it is evident to the man skilled in the art that dosages would be determined by the attending physician, according to the disease to be treated, the method of administration, the patient's age, weight, contraindications and the like.

The principles of the present invention will be more fully understood in the following examples, which are to be construed in a non-limitative manner.

### EXAMPLES

### Synthetic Examples

Synthesis of Compound A: (−)-4-[4-(1,1-Dimethyl-heptyl)2,6-dihydroxy-phenyl]-6,6-dimethyl-bicyclo[3.1.1 )heptan-2-one.

The synthesis of compound A is depicted in Scheme 1 when the R moiety of the resorcinol compound is 1,1-dimethyl-heptyl.

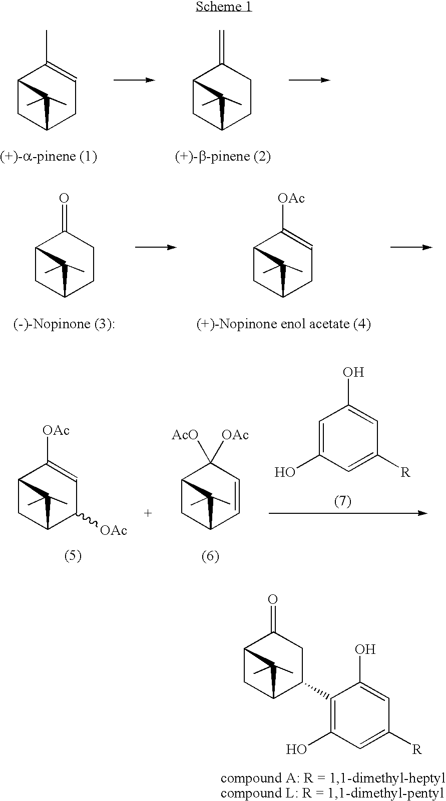

To a 3-necked flask containing n-butyl lithium (196 ml, 2M) and 44 g potassium tert-butoxide at −78° C. under nitrogen atmosphere, 50 ml of (+)-α-pinene (1) was added dropwise. The reaction was allowed to warm up to room temperature and was stirred continuously for 48 hours. The reaction was then cooled to −78° C. Trimethyl borate (113 ml) in 80 ml of ether was added and the reaction was allowed to warm up to room temperature and was stirred for one additional hour. The organic layer was separated, and the aqueous layer was extracted with n-hexane (3×80 ml). The combined organic phases were washed with brine, dried over anhydrous sodium sulfate, filtered and evaporated to dryness to afford compound (2), (+)-β-pinene. This procedure is according to Brown et al. (Brown H. C. et al., J. Org. Chem. 54: 1764-6, 1989). To (+)-β-pinene (2) (30.8 g) were added RuCl₃ (0.470 g), and benzyltributyl ammonium chloride (2.12 g) dissolved in 250 ml of ethyl acetate. To this mixture, sodium periodate (145.5 g) in 1.3 L of water was added dropwise, stirred at room temperature for 3 hours and left overnight. 250 ml of ethyl acetate were added to the reaction mixture. The organic phase was separated, washed with 500 ml of brine, 500 ml of 10% sodium sulfite, dried over anhydrous sodium sulfate, filtered, evaporated under reduced pressure to afford compound (3), (−)-Nopinone. This procedure is according to Yuasa et al. (Yuasa Y. et al., J. Essent. Oil. Res. 10: 39-42, 1998). (−)Nopinone (3) (14.86 g) and p-toluenesulfonic acid (1.48 g) were dissolved in isoprenyl acetate (148 ml). The reaction mixture was heated at reflux for 5 hours using a Dean-Stark apparatus to remove the acetone. The solvents were removed under reduced pressure, and the residue was taken in 400 ml of ether, washed with water, dried over anhydrous sodium sulfate, filtered and evaporated to afford compound (4), (+)-Nopinone enol acetate. This procedure is based on a method developed for the opposite enantiomer by Archer et al. (Archer R. A. et al., J. Org. Chem. 42: 2277-84, 1977). To a solution of 16.17 g of (+)Nopinone enol acetate (4) in 202 ml of dry toluene were added 62.2 g of Pb(OAc)₄ (previously dried in vacuo over P₂O₅/KOH overnight). The reaction mixture was heated at 80° C. for 3.5 hours, cooled, filtered, washed with saturated sodium bicarbonate. The organic layer was separated, dried over anhydrous sodium sulfate and evaporated under reduced pressure to yield (+)-6,6-Dimethyl-2,4-diacetoxy-2-norpinene (5) and (−)-6,6-dimethyl-2,2-diacetoxy-3-norpinene (6). A mixture of 5 and 6 (1.18 g, 5 mmol), resorcinol wherein R is 1,1-dimethylheptyl (7) (1.18 g, 5 mmol) and p-toluenesulfonic acid (0.95 g, 5 mmol) in chloroform (50 ml) was allowed to react at room temperature for 4 hours. Ether (30 ml) was then added, and the organic phase was washed with saturated sodium bicarbonate, water, then dried over anhydrous sodium sulfate, filtered and evaporated. The residue was allowed to crystallize in acetonitrile to provide 0.5 g of crystals. The mother liquors were chromatographed over silica gel to afford further 0.7 g of pure compound A.

Synthesis of Compound L: (−)-4-[4(1,1-Dimethyl-pentyl)-2,6-dihydroxy-phenyl]-6,6-dimethyl-bicyclo[3.1.1]heptan-2-one.

The synthesis of compound L is depicted in Scheme 1 when the R moiety of the resorcinol compound is 1,1-dimethyl-pentyl. Compounds 1 to 6 were prepared as described for the synthesis of compound A.

Synthesis of Compound B: (−)-4-[4-(1,1-Dimethyl-heptyl)-2,6-dimethoxy-phenyl]-6,6-dimethyl-bicyclo[3.1.1]heptan-2-one.

The synthesis of compound B is depicted in Scheme 2.

To a solution of compound A (115 mg, 0.3 mmol) in DMF (5 ml) was added potassium carbonate (0.5 g, 3.6 mmol) and the mixture was stirred for 10 minutes. Iodomethane (0.15 ml, 24 mmol) was then added and the mixture was stirred overnight at room temperature. Water was added to the reaction mixture and extracted with EtOAc. The organic phase was washed twice with water, dried over anhydrous sodium sulfate and evaporated. The residue was chromatographed over reversed phase C-18 column using 10% water in acetonitrile as the eluent to afford 98 mg of compound B. 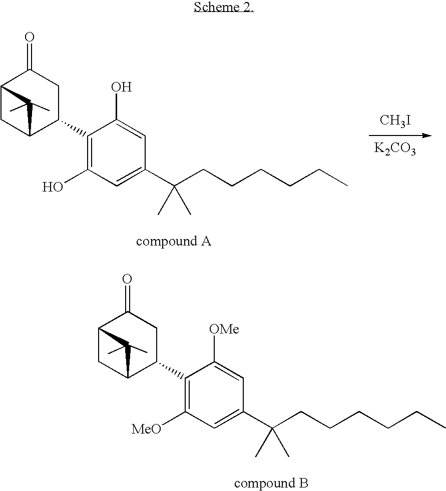
Synthesis of Compound C: (−)-5-(1,1-Dimethyl-heptyl)-2-(4-hydroxy-6,6-dimethylbicyclo[3.1.1]hept-2-yl)-benzene-1,3-diol.

The synthesis of compound C is depicted in Scheme 3.

100 mg of compound A dissolved in 10 ml of methanol were cooled to 0° C. Sodium borohydride (200 mg) was added portionwise and the reaction mixture was stirred for 4 hours. The mixture was poured into 50 ml of 5% HCl, extracted with ethyl acetate (2×30 ml), dried over Na₂SO₄, filtered and evaporated to give 90 mg of compound C in the form of white powder. 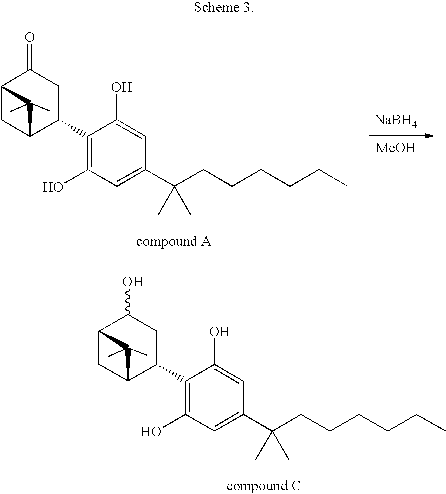
Synthesis of Compound D: 4-[4-1,1-Dimethyl-heptyl)2,6-dihydroxy-phenyl]-6,6-dimethyl-bicyclo[3.1.1]heptan-2-one Oxime.

The synthesis of compound D is depicted in Scheme 4.

Hydroxylamine hydrochloride (37.3 mg) was dissolved in 5 ml of water and the solution cooled to 0° C. Potassium hydroxide (30 mg) in 1 ml of water was added slowly. Compound A (372.5 mg) was added followed by addition of methanol to dissolve all the components. After 3 hours of stirring, no starting material could be observed. Water was then added and the solution was extracted with ethyl acetate, dried over Na₂SO₄, filtered and evaporated to afford 380 mg of compound D. 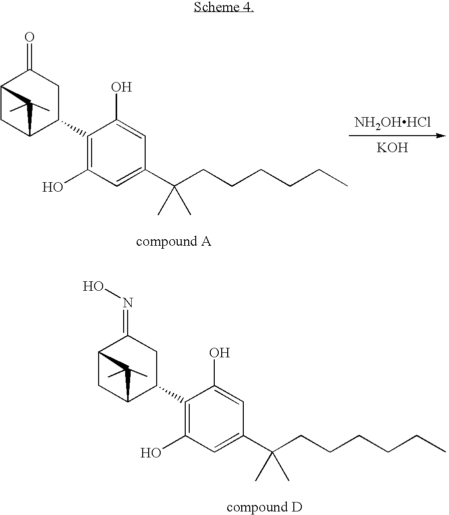
Synthesis of Compound E: (+)-5-(1,1-Dimethyl-hept-6-ynyl)-2-(4,6,6-trimethyl-bicyclo[3.1.1]hept-3-en-2-yl)-benzene-1,3-diol.

The synthesis of compound E is depicted in Scheme 5 when the R moiety of the resorcinol compound is 1,1-dimethyl-hept-6-ynyl.

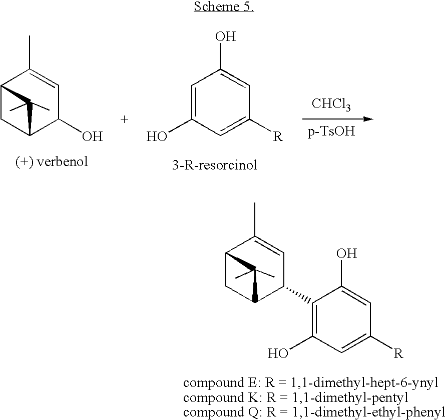

The reaction was carried out under anhydrous conditions. A well-stirred mixture of (+) verbenol (0.505 g, 3.3 mmol), 3-(1,1-dimethylhept-6-ynyl) resorcinol (0.77 g, 3.3 mmol) and catalytic amount of anhydrous p-toluenesulfonic acid in dry chloroform (10 ml) were stirred at 0° C. for 1 hour. The mixture was poured onto an aqueous solution of sodium bicarbonate (50 ml) and the aqueous phase was extracted with chloroform (3×30 ml). The combined organic layers were then washed with water (3×30 ml), and brine (3×100 ml). The organic layer was dried over Na₂SO₄, filtered and evaporated. The crude material thus obtained was purified by flash chromatography on silica gel using 5% ether/petroleum ether as the eluent to afford 1.034 g of compound E.

Synthesis of Compound Q: 5-(1,1-Dimethyl-ethyl-phenyl)-2-(4,6,6-trimethyl-bicyclo[3.1.1]hept-3-en-2-yl)-benzene-1,3-diol.

The synthesis of compound Q is depicted in Scheme 5 wherein the R moiety of the resorcinol compound is 1,1-dimethyl-ethyl-phenyl.

1,1-dimethyl-ethyl-phenyl resorcinol was prepared as described for compound 14 using phenyl lithium. The condensation with (+)-verbenol was performed as described for compound E.

The Synthesis of Compound F: (−)-4-[4-1,1-Dimethyl-hept-6-ynyl)-2,6-dihydroxy-phenyl]-6,6-dimethyl-bicyclo[3.1.1]heptan-2-one.

The synthesis of compound F is depicted in Scheme 6.

(3,5-Dimethoxyphenyl)-N-methoxy-N-methylcarboxamide (8) was prepared as described by Harrington et al. (Harrington P. E. et al., J. Org. Chem. 65: 6576-82, 2000). 1-(trimethylsilyl)-6-bromo-1-hexyne (9) was prepared according to Negishi et al. (Negishi E-I et al., J. Amer. Chem. Soc. 110: 5383-96, 1988). [7-(3,5-Dimethoxyphenyl)-7-oxo-1-heptynyl]trimethyl silane (10) was prepared according to the following procedure.

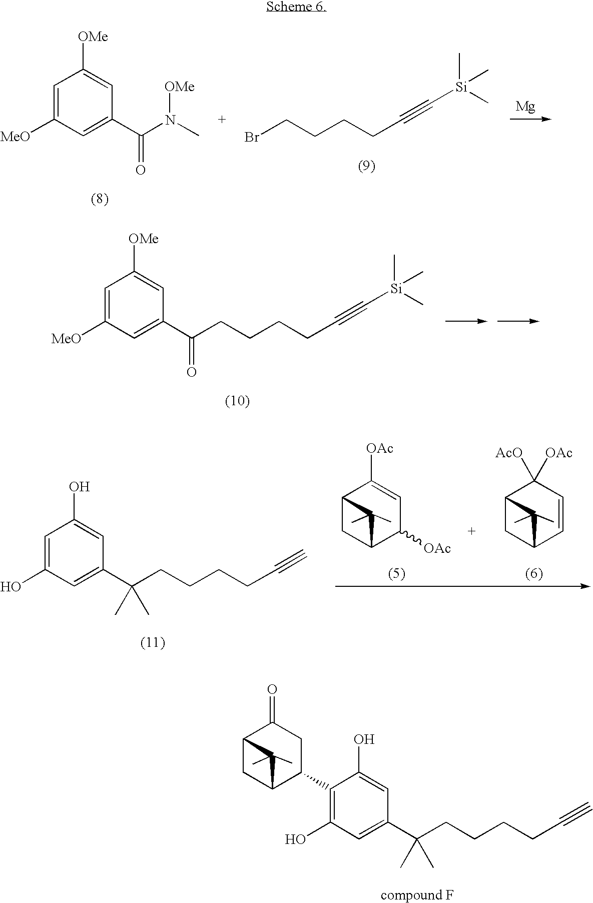

To magnesium metal (300 mg) in 5 ml of anhydrous THF, a catalytic amount of dibromomethane was added and the reaction mixture was heated to reflux for a few minutes. The heating was stopped and 0.9 ml of compound 9 were injected using a syringe at an addition rate that maintained reflux (ca 20 min). After the addition was complete, reflux was continued for an additional hour. The reaction mixture was cooled to room temperature. The Grignard thus obtained was transferred via cannula to a solution compound 8 (0.9 g) in 2 ml of THF at 0° C. After 30 min, the reaction mixture was quenched with 1M HCl solution and diluted with ether. The organic phase was separated, dried over Na₂SO₄, filtered and evaporated to afford 1.5 g of crude material. Purification by flash chromatography on silica gel using 10% ethyl acetate in petroleum ether as the eluent gave 680 mg of pure compound 10. 3-(1,1-Dimethyl-6-ynyl) resorcinol (11) was obtained from compound 10 as described in the international patent application WO 01/28497. A mixture of 5 and 6 (1.18 g, 5 mmol), 3-(1,1-Dimethyl-6-ynyl) resorcinol (11) (1.18 g, 5 mmol) and p-toluenesulfonic acid (0.95 g, 5 mmol) in chloroform (50 ml) was allowed to react at room temperature for 4 hours. Ether (30 ml) was then added, and the organic phase was washed with saturated sodium bicarbonate, water, then dried over anhydrous sodium sulfate, filtered and evaporated. The residue was allowed to crystallize in acetonitrile to provide 0.5 g of crystals. The mother liquors were chromatographed over silica gel to afford further 0.7 g of pure compound F.

The Synthesis of Compound G: 4-[4-(1,1-Dimethyl-3-phenyl-propyl)-2,6-dihydroxy-phenyl]-6,6-dimethyl-bicyclo[3.1.1]heptan-2-one.

The synthesis of compound G is depicted in Scheme 7 when R is 2-ethyl-benzene.

Compounds 8, 12 and 13 were prepared as described by Harrington et al. (Harrington P. E. et al., J. Org. Chem. 65: 6576-82, 2000). Compound 14 was prepared as described in the international patent application WO 01/28497. Compounds 5 and 6 were prepared as previously described in the synthesis of compound A. The condensation of compounds 5, 6 and 14 was performed as described for the synthesis of compound A. 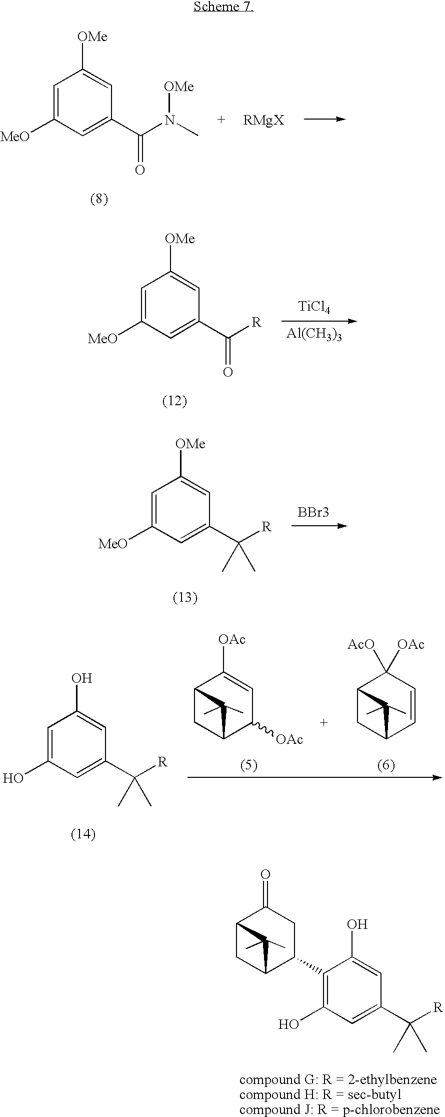
The Synthesis of Compound H: (−)-4-[2,6-Dihydroxy-4-(1,1,3-trimethyl-butyl)-phenyl]-6,6-dimethyl-bicyclo[3.1.1]heptan-2-one.

The synthesis of compound H is depicted in Scheme 7 when R is sec-butyl. Compounds 5, 6, 8, 12-14 were prepared as described for the synthesis of compounds A and F. The condensation of compounds 5, 6 and 14 was performed as described for the synthesis of compound A.

The Synthesis of Compound J: (−)-4-{4-[1-(4-Chloro-phenyl)-1-methyl-ethyl]-2,6-dihydroxy-phenyl]-6,6-dimethyl-bicyclo[3.1.1]heptan-2-one.

The synthesis of compound J is depicted in Scheme 7 when R is p-chlorobenzene.

Compounds 5, 6, 8, 12-14 were prepared as described for the synthesis of compounds A and F. The condensation of compounds 5, 6 and 14 was performed as described for the synthesis of compound A.

The Synthesis of Compound M: (−)-4-[4-(1-Ethyl-1-methyl-propyl)-2,6-dihydroxy-phenyl]-6,6-dimethyl-bicyclo[3.1.1]heptan-2-one.

The synthesis of compound M is depicted in Scheme 8.

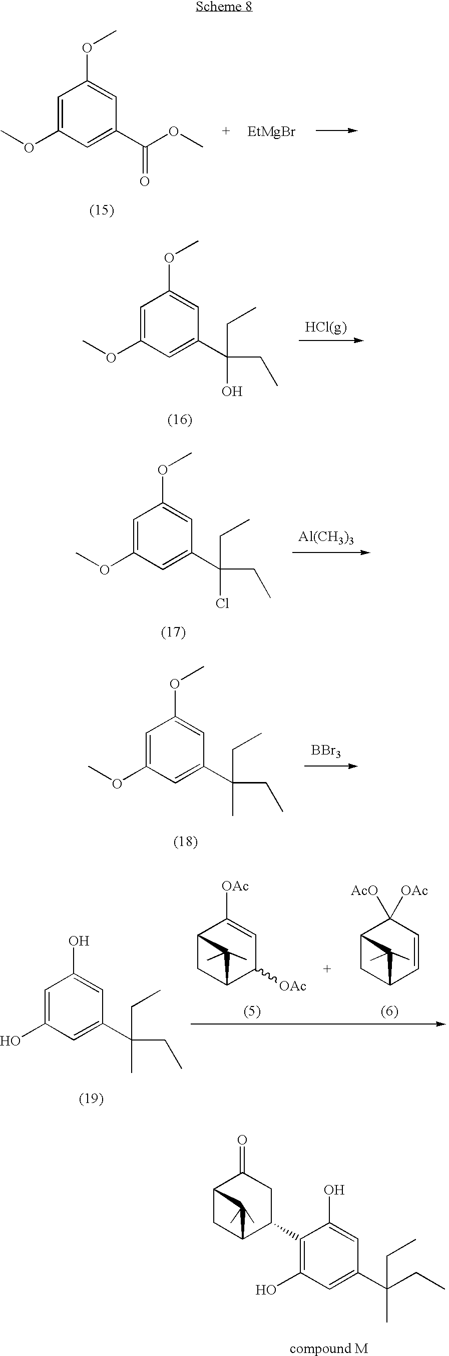

Synthesis of compound 16, 1-(1-hydroxy-1-ethyl-propyl)-3,5-dimethoxy-benzene, was carried out as follows. Reaction was performed under anhydrous conditions. To a solution of Methyl-3,5-dimethoxy benzoate (5 g, 25.5 mmole) in dry THF (100 ml) at 0° C., Ethyl-magnesium bromide (1M in THF, 76.5 ml) was added. The reaction mixture was stirred 72 hours at room temperature. Ethyl acetate and water were added and the aqueous layer was extracted with ethyl acetate. The combined organic layers were then washed with water and brine, dried (Na₂SO₄), and filtered to afford 6.3 g of compound 16.

The following procedures were described in international patent application WO 01/28497. Synthesis of compound 17, 1-(1-chloro-1-ethyl-propyl)-3,5-dimethoxy-benzene, was carried out as follows. Compound 16 (6.3 g, 25 mmole) was dissolved in anhydrous CCl₄ (30 ml) an HCl (g) was bubbled through for 1 hour. The organic layer was washed with water and 10% sodium bicarbonate solution, dried (Na₂SO₄) and evaporated to give 6.3 g of compound 17. Synthesis of compound 18, 1-(1-ethyl-1-methyl-propyl)-3,5-dimetoxy-benzene, was carried out as follows. A solution of compound 17 (6 g, 25 mmole) in dry toluene was cooled to −30° C. under N₂ and trimethylaluminum (2M solution in heptane) (25 ml) was added. The reaction mixture was allowed to warm to room temperature and was stirred overnight. HCl (1N) was added, the organic layer was then separated, washed with water, dried and evaporated. The crude material was chromatographed on silica gel using 1% ethyl acetate/petroleum ether as the eluent to afford 5.3 g of compound 18.

Synthesis of compound 19, 5-(1-ethyl-1-methyl-propyl)-resorcinol, was carried out as follows. To a cooled (−50° C.) solution of compound 18 in dry dichloromethane, boron-tri-bromide (10.15 ml, 107.3 mmole) was added under N₂ atmosphere. The reaction mixture was allowed to warm to room temperature and stirred overnight. Saturated sodium bicarbonate was added, the organic layer was separated, dried (Na₂SO₄) and evaporated to give 4.2 g of the desired resorcinol 19. Compounds 5 and 6 and the condensation with the resorcinol 19 were prepared as described for the synthesis of compound A.

The Synthesis of Compound N: (−)-4-[4-(5-Bromo-1,1-Dimethyl-pentyl)-2,6-dihydroxy-phenyl]-6,6-dimethyl-bicyclo[3.1.1]heptan-2-one.

The synthesis of compound N is depicted in Scheme 9 when R is a bromine atom.

Compounds 5 and 6 were prepared as described for the synthesis of compound A. Compound 20 was prepared as described by Singer et al. (Singer et al. J. Med. Chem. 41: 4400-7, 1998). The condensation of compounds 5, 6 and 20 was performed as described for the synthesis of compound A.

The Synthesis of Compound P: (−)-4-[4-(1,1-Dimethyl-pentyl-5-nitrile)-2,6-dihydroxy-phenyl]-6,6-dimethyl-bicyclo[3.1.1]heptan-2-one.

The synthesis of compound P is depicted in Scheme 9 when R is a cyano group.

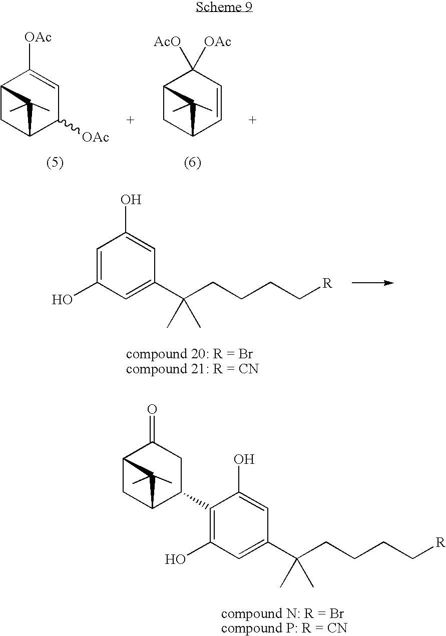

Compounds 5 and 6 were prepared as described for the synthesis of compound A. Compound 21 was prepared from compound 20 in a procedure similar to one described by Singer et al. (Singer et al., ibid). The condensation of compounds 5, 6 and 21 was performed as described for the synthesis of compound A.

The Synthesis of Compound R: (−)-4-{4-[1,1-Dimethyl-heptyl]-2-succinate-6-hydroxy-phenyl}-6,6-dimethyl-bicyclo[3.1.1]heptan-2-one.

The synthesis of compound R is depicted in Scheme 10.

The Synthesis of Compound S: 4-{4-[1,1-Dimethyl-heptyl]-2,6-bisuccinate-phenyl}-6,6-dimethyl-bicyclo[3.1.1]heptan-2-one.

The synthesis of compound S is depicted in Scheme 10.

A mixture of compound A (227 mg, 0.61 mmole) and succinic anhydride (731 mg, 7.31 mmole) in dry pyridine (10 ml) was heated to 50° C., under N₂ atmosphere. Potassium t-butoxide was added and the obtained mixture was stirred overnight (50° C.). The mixture was poured into 1N HCl, and extracted with ethyl acetate. The combined organic phase was washed with 1N HCl and brine, dried (Na₂SO₄) and evaporated. The two products were separated by column chromatography (20% ethyl acetate/petroleum ether+0.1% acetic acid) to yield 220 mg of compound R (oil) and 150 mg of compound S (solid). 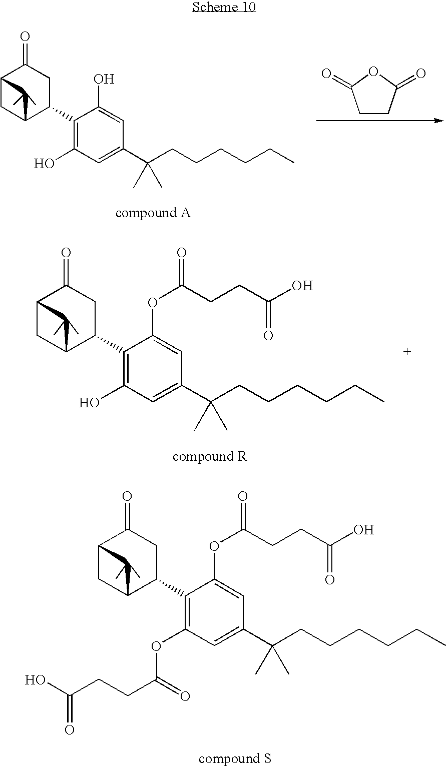
The Synthesis of Compound T: 4-{4-[1,1-dimethyl-heptyl]-2,6-bi-diethylphosphate-phenyl]-6,6-dimethyl-bicyclo[3.1.1]heptan-2-one.

The synthesis of compound T is depicted in Scheme 11.

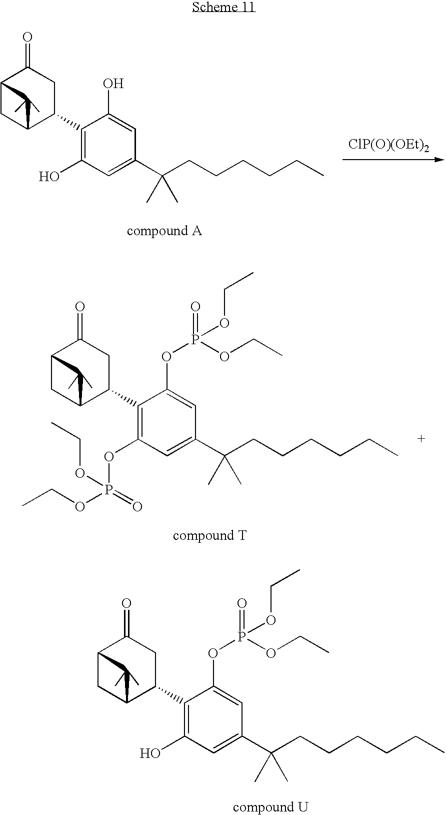

Reaction was carried out under N₂ atmosphere. To a well stirred solution of compound A (1.97 g, 5.29 mmole) in freshly distilled THF, potassium t-butoxide (1.54 g, 13.75 mmole) was added and the mixture was stirred for 10 minutes. Diethyl chlorophosphate was added then and the reaction mixture was stirred overnight. Water was added and the aqueous phase was extracted with ethyl acetate. The combined organic layers were washed with brine, dried (Na₂SO₄) and evaporated. Purification by chromatography on silica-gel using 25%-70% ethyl acetate-petroleum ether as eluent gave 2.2 g of pure compound T.

The Synthesis of Compound U: 4-{4-[1,1-dimethyl-heptyl]-2-diethylphosphate-6-hydroxy-phenyl]-6,6-dimethyl-bicyclo[3.1.1]heptan-2-one.

The synthesis of compound U is depicted in Scheme 11.

The Synthesis of Compound W: (−)-4-{4-[1,1-dimethyl-heptyl]-2,6-bi-diethylphosphate-phenyl}-6,6-dimethyl-bicyclo[3.1.1]heptan-2-methylene.

The synthesis of compound W is depicted in Scheme 12.

The synthesis was carried out under N₂ atmosphere. To a suspension of methyl-triphenyl-phosphonium iodide (5.92 g, 14.65 mmole) in anhydrous THF (100 ml), potassium bis(trimethylsilyl)amide (PBTSA) (0.5 M in toluene, 28.7 ml) was added and the mixture was stirred 0.5 hour at room temperature. A solution of compound T (1.89 g, 2.93 mmole) in THF (10 ml) was added then and the mixture was stirred overnight. An aqueous solution of ammonium chloride was added, the organic layer was separated, and the aqueous layer was extracted with EtOAc. The combined organic phase was washed with brine, dried (Na₂SO₄), filtered and evaporated. The product was purified by chromatography on silica gel column using 15% to 30% EtOAc/Petroleum ether as eluent. 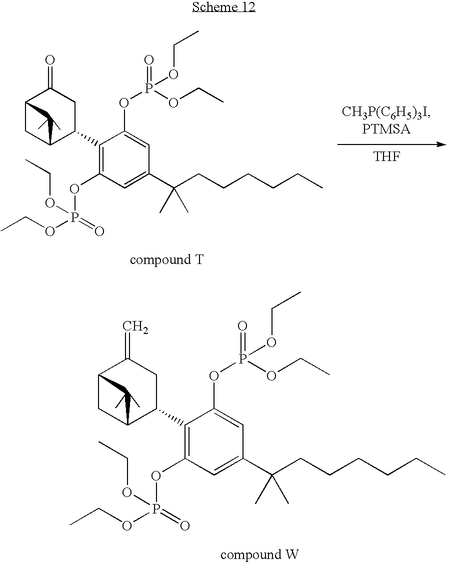
The Synthesis of Compound Y: (−)-4-{4-[1,1-Dimethyl-pentyl]-2-succinate-6-hydroxy-phenyl}-6,6-dimethyl-bicyclo[3.1.1]heptan-2-one.

The synthesis of compound Y is similar to the synthesis of compound R depicted in Scheme 10. The only difference resides in the starting material, while compound A yields compound R, compound L yields compound Y using the same synthetic procedure.

The Synthesis of Compound Z: (−)-4-{4-[1,1-Dimethyl-heptyl]-2-fumarate-6-hydroxy-phenyl}-6,6-dimethyl-bicyclo[3.1.1]heptan-2-one.

The synthesis of compound Z is depicted in Scheme 13.

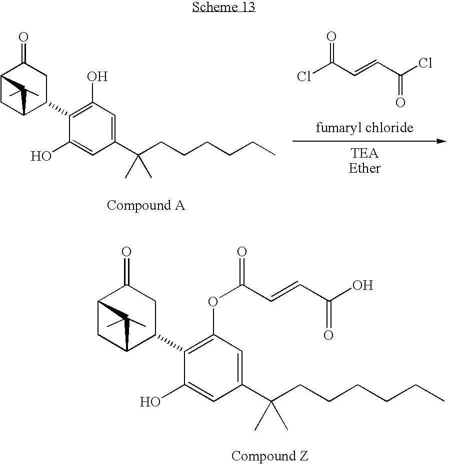

Compound A (600 mg, 1.6 mmol) was dissolved in 100 ml of dry diethyl ether. Then 0.21 ml of triethylamine (1.6 mmol) was added and 0.18 ml of fumaryl chloride (1.7 mmol). After stirring for about 15 minutes, the salt trimethylammoniun chloride was filtered and the filtrate was evaporated. Then ethyl acetate was added to the residue and washed three times with water until the pH was above 4. The organic phase was then washed with saturated sodium chloride, dried over sodium sulfate, filtered and evaporated. Compound Z was then purified by column chromatography on silica gel using 20% ethyl acetate and petroleum ether as eluent.

Synthesis of Compound AA: (−)-4-[4-(1,1-Dimethyl-heptyl)-2-hydroxy-6-methoxy-phenyl]-6,6-dimethyl-bicyclo[3.1.1]heptan-2-one.

The synthesis of compound AA is depicted in Scheme 14.

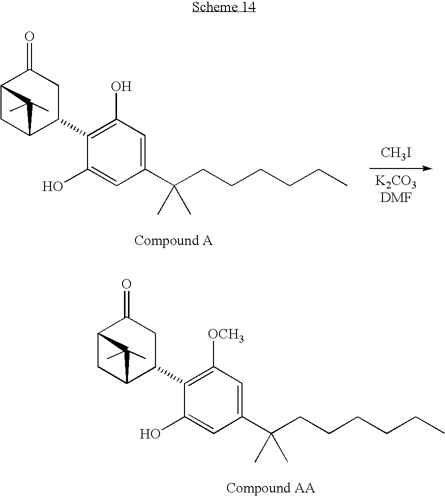

To a solution of compound A (150 mg, 0.4 mmol) in DMF (16 ml) was added potassium carbonate (400 mg, 2.9 mmol) and the mixture was stirred for 10 min at room temperature. Iodomethane (85.2 mg, 0.6 mmol) was then added and the mixture was stirred overnight at room temperature. Water was added to the reaction mixture and extracted with EtOAc. The organic phase was washed twice with water, dried over anhydrous sodium sulfate, filtered and evaporated. The residue was chromatographed over reverse phase column using 50% water in acetonitrile as eluent to afford 30 mg of compound AA.

The Synthesis of Compound AB: 4-{4-[1,1-dimethyl-heptyl]-2,6-bi-diethylphosphate-phenyl}-6,6-dimethyl-bicyclo[3.1.1]heptan-2-ol.

The synthesis of compound AB is depicted in Scheme 15.

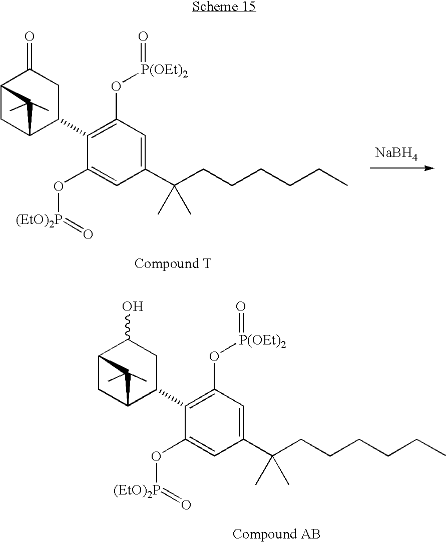

To a well cooled solution (−50° C.) of compound T (0.12 g, 0.18 mmol) in dry ethyl alcohol (6 ml), sodium borohydride (51 mg, 1.34 mmol) was added. The reaction mixture was stirred at −40° C. for 1 hour, and then allowed to warm up to room temperature. After three hours, TLC analysis indicated the complete disappearance of starting material. Water was then added and the reaction mixture was extracted with ethyl acetate. The organic layer was washed with water, saturated sodium chloride and dried over sodium sulfate. The remaining solvent was removed by evaporation to afford 0.17 g of compound AB (92% yield).

The Synthesis of Compound AC: 4-[4-(1,1-Dimethyl-heptyl)-phenyl]-6,6-dimethyl-bicyclo[3.1.1]heptan-2-ol.

The synthesis of compound AC is depicted in Scheme 16.

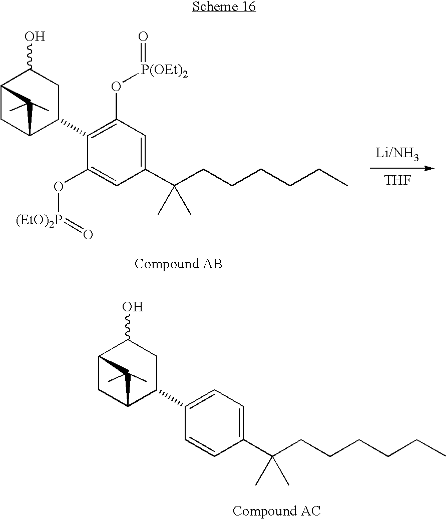

The reaction was conducted under anhydrous conditions. To a well cooled (−78° C.) solution of compound AB (0.107 g, 0.165 mmol) in anhydrous THF (6 ml) and liquid ammonia (˜50 ml), lithium (˜50 mg, 7.2 mmol) was added. The reaction vessel was maintained fully closed until the blue color disappeared (about 30 minutes) and then left open for overnight to let the ammonia evaporate. The residue was dissolved in ethyl acetate (30 ml) and a saturated solution of ammonium chloride. The aqueous phase was extracted with ethyl acetate. The combined organic phases were dried over sodium sulfate, filtered and evaporated, to afford 77.6 mg of compound AC, 100% pure according to HPLC.

Synthesis of Compound AD: (−)-4-[4-(1,1-Dimethyl-heptyl)-phenyl]-6,6-dimethyl-bicyclo[3.1.1]heptan-2-one.

The synthesis of compound AD is depicted in Scheme 17.

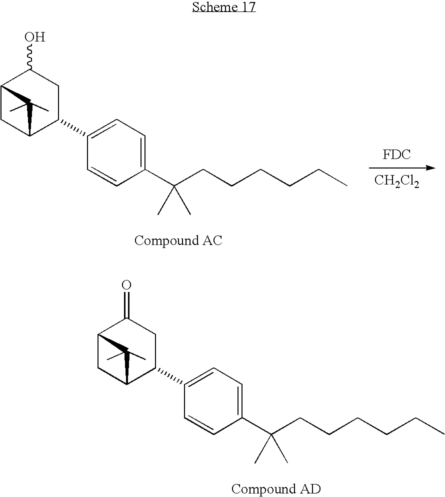

To a well-stirred solution of compound AC (0.204 g, 0.6 mmol) in anhydrous dichloromethane, pyridinium dichromate (0.448 g, 1.2 mmol) was added in one portion. The reaction mixture was stirred at room temperature overnight. The solids were filtered through celite, and washed with DCM. The solvent was removed by evaporation to afford a residue of 0.27 g. The crude material was purified by flash chromatography using 10% ethyl acetate/petroleum ether as eluent to afford 0.15 g of pure compound AD (yield 74%).

Synthesis of Compound AE: (+)-5-(Methyl Ester Pentanoic Acid)-2-(4,6,6-trimethyl-bicyclo[3.1.1]hept-3-en-2-yl)-benzene-1,3-diol.

The synthesis of compound AE is depicted in Scheme 18.

A solution of methyl 3,5-dimethoxybenzoate (20 g, 0.12 mole), imidazole (100 g, 1.47 mole), and tert-butyldimethylsilyl chloride (100 g, 0.66 mole) in DMF (anhydrous, 400 ml) was stirred at room temperature for 2 hours. The reaction mixture was diluted with water (300 ml) and the aqueous layer was extracted with ether (3×300 ml). The combined organic phases were washed with water, dried (sodium sulfate) and evaporated. The crude material obtained was dissolved in THF (300 ml), cooled to −20° C. and LiAlH₄ (1N in THF, 140 ml, 0.14 mole) was added dropwise. The reaction mixture was stirred at room temperature for 2 hours. The reaction mixture was cooled to −30° C. and ethyl acetate was added (300 ml) followed by a saturated solution of MgSO₄. The solution obtained was filtered through celite.

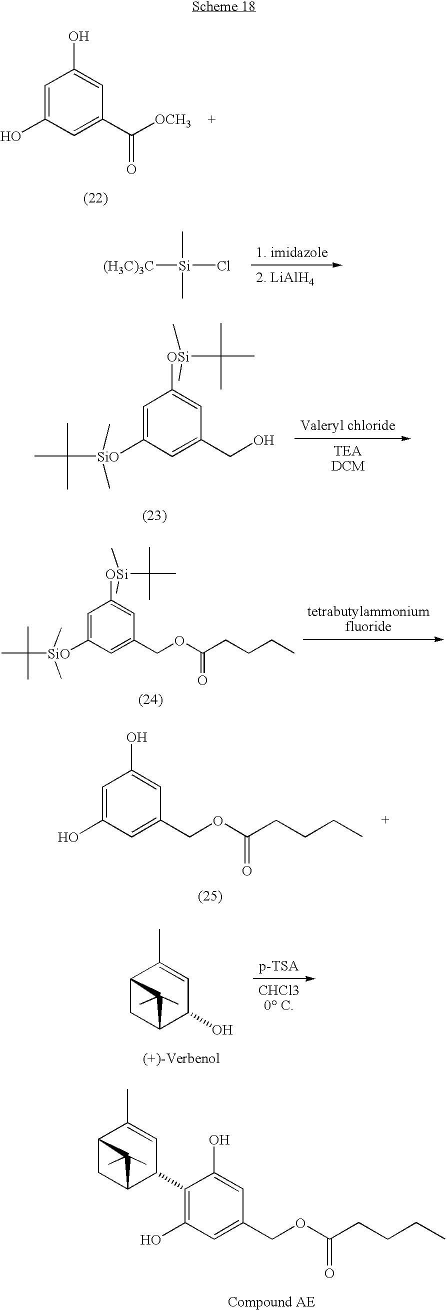

The organic layer was separated and the aqueous phase was extracted three times with ethyl acetate. The combined organic phases were washed with saturated sodium chloride, dried (sodium sulfate) and evaporated, to afford 44.2 g of crude product (23) (0.12 mole). Without any purification steps, triethylamine (25 ml, 0.18 mole) and valeryl chloride (30 ml, 0.25 mole) were added to the crude product (23) dissolved in dry dichloromethane (1 liter). The resulting mixture was stirred overnight at room temperature. Water was then added, the organic layer was separated, and the aqueous phase was extracted with DCM (3×300 ml). The combined organic phases were washed with saturated sodium chloride, dried (sodium sulfate) and evaporated. The residue was chromatographed on silica gel with 4% ethyl acetate/petroleum ether as eluent. 45 g of yellow oil was obtained (24). To the yellow oil (45 g, 0.1 mole) in THF (1 liter), tetrabutylammonium fluoride (87 g, 0.33 mole) was added and the mixture was stirred overnight at room temperature. The reaction mixture was poured into water (1 liter) acidified with acetic acid until pH ˜4.5 and extracted several times with ethyl acetate. The combined organic phases were washed with saturated sodium chloride, dried (sodium sulfate) and evaporated. The residue was chromatographed on silica gel column with 30% ethyl acetate and petroleum ether as eluent to afford 20 g of off-white solid (25). The off-white solid (0.75 g, 3.3 mmol) was dissolved with (+)-verbenol (0.5 g, 3.3 mmol) in CHCl₃ (40 ml) and the resulting solution was cooled to 0° C. Anhydrous p-toluenesulfonic acid (catalytic amount) was added and the resulting mixture was stirred at 0° C. for 15 minutes. The reaction mixture was poured into a saturated solution of sodium carbonate. The aqueous phase was extracted with CHCl₃ and the combined organic phases were washed with aqueous solution of sodium carbonate. The organic phase was dried (sodium sulfate), filtered and evaporated. Compound AE was isolated and purified by preparative HPLC with 20% water with acetonitrile as eluent.

Synthesis of Compound AF: 4-{4-[1,1-Dimethyl-heptyl]-2,6-dimethoxy-phenyl}-6,6-dimethyl-bicyclo[3.1.1]heptan-2-methylene.

The synthesis of compound AF is depicted in Scheme 19.

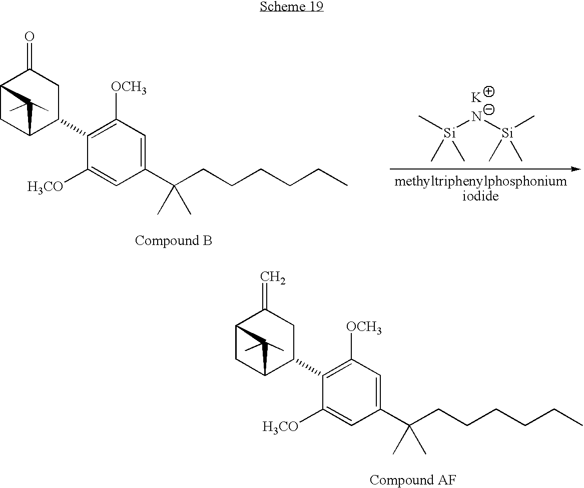

The reaction was performed under N₂ atmosphere and anhydrous conditions. To a suspension of methyltriphenylphosphonium iodide (1.083 g, 2.68 mmol) in dry THF (20 ml), potassium bis (trimethylsilyl)amide ( 5.26 ml, 2.63 mmol, 0.5 M in toluene) was added. The mixture was stirred for half an hour at room temperature. Then a solution of compound B (0.214 g, 0.537 mmol) in dry THF (2 ml) was added, and the resulting mixture stirred overnight. A saturated solution of ammonium chloride was added to the reaction mixture and the aqueous phase was extracted with ethyl acetate (3 times), the combined organic phases were washed with saturated sodium chloride, dried (sodium sulfate) filtered and evaporated. A crude brown solid that was obtained titurated with hexane in order to remove triphenylphosphine oxide. Then it was chromatographed on silica gel with 100% hexane as eluent, to obtain compound AF as a light yellow oil.

Synthesis of Compound AG: (−)-4-[4-(1,1-Dimethyl-pent-4-enyl)-2,6-dihydroxy-phenyl]-6,6-dimethyl-bicyclo[3.1.1]heptan-2-one.

The synthesis of compound AG is depicted in Scheme 20.

One gram of sodium metal (43 mmol) was dissolved in dry methyl alcohol (25 ml), then 4-(7-Bromo-1,1-Dimethyl-heptyl)-2,6-dihydroxy-phenyl (20) dissolved in methanol (3.5 g, 12 mmol) was added. The reaction mixture was stirred for about half an hour. Then the reaction mixture was poured into 100 ml of 1 N HCl. The aqueous phase was extracted (3×100 ml) with ethyl acetate, the combined organic phases were washed with saturated sodium chloride, dried (sodium sulfate) filtered and evaporated. 800 mg of crude resorcinol product (26) was obtained which was purified by column chromatography on silica gel with 20% ethyl acetate in petroleum ether. The product resorcinol (170 mg, 0.82 mmol) was allowed to react with two isomers of nopinone di-acetates, (5) and (6), (540 mg, 2.2 mmol) in CHCl₃ with catalytic amount of p-toluenesulfonic acid. After stirring at room temperature for 4 hours, the reaction was completed. The reaction mixture was washed with sodium bicarbonate and extracted with ethyl acetate (3 times), the combined organic phases were washed with saturated sodium chloride, dried (sodium sulfate) filtered and evaporated. The obtained crude material was titurated with petroleum ether to give 150 mg of crude compound AG. The product was then purified by reverse phase chromatography with 50% water acetonitrile as eluent. 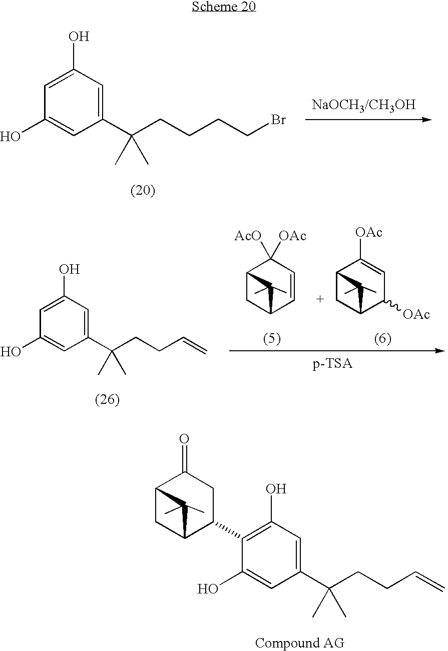
Synthesis of Compound AH: 2,2-dimethylpropionic Acid-4-{4-[1,1-Dimethyl-pentyl]-2,6-dihydroxy-phenyl}-6,6-dimethyl-bicyclo[3.1.1]hept-2-en-2-yl Methyl Ester.

The synthesis of compound AH is depicted in scheme 21.

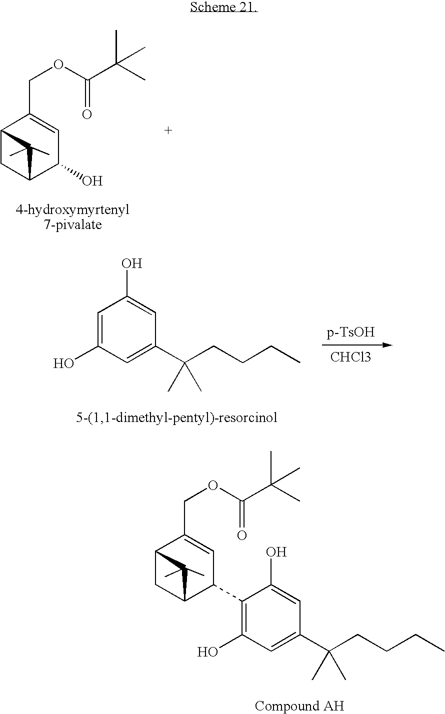

4-hydroxymyrtenyl-pivalate was prepared as described in U.S. Pat. No. 4,876,276 and 5-(1,1-dimethylpentyl)-resorcinol was prepared as follows. In a 250 ml round bottom flask 100 ml of methanol and 100 ml of THF were added. Then 5.5 g of 3,5-dimethoxy-benzoic acid (0.03 mole) and 1.27 g of lithium hydroxide monohydrate (0.03 mole) were added. Then 10 ml of water was added and the reaction mixture was stirred for 1 hour. The slurry obtained was filtered and evaporated. The residue was titurated with ether and evaporated again to obtained yellowish solid. The solid dried with P₂O₅ under reduced pressure at 60° C. The dried salt of 3,5-dimethoxy lithium benzoate was added to a 250 ml round bottom flask filled with 100 ml of THF. N-butyl lithium (20 ml, 1.7 M, 0.032 mole) was added. The reaction was warmed up to 50° C. and stir for two hours. Then the reaction mixture was cooled to room temperature and added dropwise to 250 ml of 1 N HCl. Then Na₂CO₃ was added until pH ˜11. Then the reaction mixture was extracted 3 times with ether. The combined organic phases were dried over sodium sulfate filtered and evaporated to give orange oil, which crystallized from n-pentane. 2.6 g of compound 12 wherein R is butyl was obtained, with an overall yield of 39%. Compounds 13 and 14 wherein R is butyl were prepared as described in scheme 7. The condensation between 4-hydroxymyrtenyl-pivalate and 5-(1,1-dimethylpentyl)-resorcinol was performed as described for compound E and the yield was 65%.

### Physiological Examples

Evaluation of the therapeutic effects of the novel bicyclic CB2 ligands was carried out in a series of experimental systems to support the utility of these drugs as immunomodulatory, anti-inflammatory, analgesic, neuroprotective and anti-tumoral agents. These effects were evaluated both in vitro and in vivo, and corroborated utilizing the systems described below. Unless otherwise indicated the test compounds are prepared as follows: for in vitro assays the compounds are first dissolved in DMSO and then stepwise diluted in the assay buffer, generally tissue culture medium, down to a final concentration of 0.1% DMSO. For in vivo assays the test compounds are first diluted in CREMOPHOR EL®:ethanol (70% and 30% w/w respectively) and further diluted 1:20 in physiological buffer, generally saline, to reach the appropriate dose. Thus the vehicle is the original “solvent” diluted in the appropriate buffer.

### Example 1

### Binding Affinity for the CB1 and CB2 Receptors

The CB1 binding assays were performed by testing the ability of the new compounds to displace [³H]CP55940 from the CB1 receptor on membranes derived from hCB1 stably transfected HEK-293 cells (Perkin Elmer/NEN). Membranes were diluted in the assay buffer (50 mM Tris-HCl, 2.5 mM EDTA, 5 mM MgCl₂, 1 mg/ml BSA, pH=7.4) to 500 μg protein/ml. 50 μl of diluted membranes (25 μg) were incubated with [³H]CP55940 in the presence or absence of the bicyclic test compounds in a total volume of 0.5 ml. Tested compounds were dissolved in DMSO and diluted in the assay buffer to a final concentration of 0.1% solvent. Control samples were added with identical amount of vehicle. Non-specific binding was measured by the addition of 10 μM of WIN 55212-2. Following 1.5 hours incubation at 30° C. reactions were filtered through Whatman 934A/H filters (presoaked with 0.1% Polyethylenimine (PEI)).

The affinities of the novel bicyclic analogs to the CB2 receptor were determined by their ability to displace [³H]WIN 55212-2 from the receptor in membranes derived from hCB2 stably transfected CHO cells (Perkin Elmer/NEN). Membranes were diluted in assay buffer (10 mM HEPES, 1 mM MgCl₂, 1 mM EDTA, 0.3 mg/ml BSA, pH=7.4) to 500 μg protein/ml. 50 μl of diluted membranes (25 μg) were incubated with 0.8 nM of [³H]WIN 55212-2 in the presence or absence of several concentrations of the bicyclic test compounds in a total volume of 1 ml. Tested compounds were dissolved and diluted as previously described for the hCB1 assay. Non-specific binding was measured by the addition of 10 μM CP 55940. Following 40 minutes incubation at 30° C. reactions were filtered as previously described. Filters for all binding assays were counted in a β-counter and log of analog concentration versus % of binding was plotted. IC₅₀ values were extrapolated from this plot.

The results of the binding assays are shown in Table 1, which depicts the Structure Activity Relationship (SAR) of the preferred compounds, in terms of their ability to displace [³H]WIN 55212-2 or [³H]CP55940 from CB2 or CB1 binding sites, respectively.

The abbreviations used in Table 1 to define R₂, R₃ and R₄ refer to the following substituents:

DMBP=1,1-Dimethyl-5-Bromo-Pentyl
DMCP=1,1-Dimethyl-5-Cyano-Pentyl
DMEP=1,1-Dimethyl-Ethyl-Phenyl
DMH=1,1-Dimethyl Heptyl
DMH6=1,1-Dimethyl Hept-6-ynyl
DMP=1,1-Dimethyl Pentyl
DMPP=1,1-Dimethyl-3-Phenyl-Propyl
EMP=1-Ethyl-1-Methyl-Propyl
MCPE=1-Methyl-1-(p-Chloro-Phenyl)-Ethyl

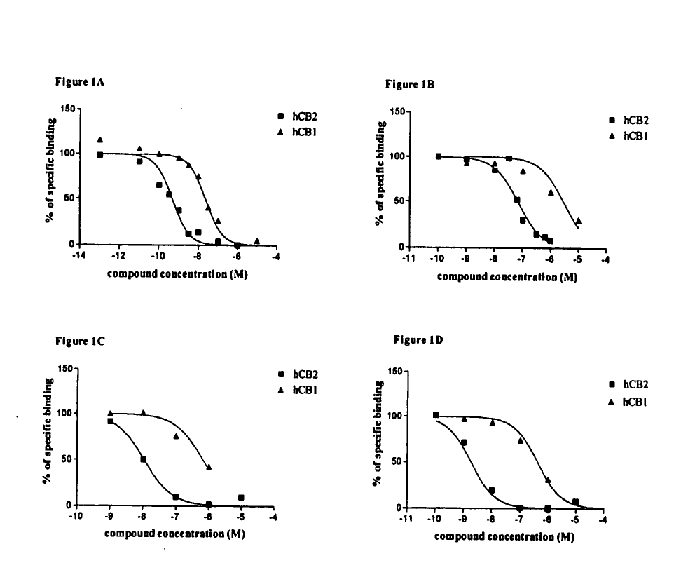

The values of IC₅₀ reported in table 1 were calculated from graphs such as depicted in FIG. 1, which shows the binding of selected bicyclic compounds to the cannabinoid receptors. Binding to CB1 is measured by competitive inhibition of [³H]CP55940 in HEK-293 cells stably transfected with the human CB1 receptor gene. Binding to CB2 is measured by competitive inhibition of [³H]WIN55212-2 in CHO cells stably transfected with the human CB2 receptor gene. Both curves (hCB1 ▪ and hCB2 ♦), representing % inhibition as a function of compound concentration, are superimposed in this graph. A—Displays the results obtained with compound A. B—Displays the results obtained with compound B. C—Displays the results obtained with compound J. D—Displays the results obtained with compound L.

TABLE 1
SAR and IC₅₀ (nM) of bicyclic compounds of formulae (I) to (III).

| | | | | | | | | CB2/CB1 |
|---|---|---|---|---|---|---|---|---|
| | | | | | | CB2 | CB1 | affinity |
| COMPOUND | R₁ | R₂ | R₃ | R₄ | R₅ | IC₅₀ | IC₅₀ | ratio |
| HU-210* | | | | | | 0.35 | 0.39 | 1.11 |
| HU-308* | | OCH₃ | OCH₃ | DMH | CH₂OH | 13.3 | 3600 | 271 |
| A | O | OH | OH | DMH | | 1 | 27.6 | 28 |
| B | O | OCH₃ | OCH₃ | DMH | | 45 | 2800 | 62 |
| C | | OH | OH | DMH | OH | 3.5 | 31 | 9 |
| D | N—OH | OH | OH | DMH | | 3.4 | 93 | 27 |
| E | | OH | OH | DMH6 | CH₃ | 0.783 | 26 | 33 |
| F | O | OH | OH | DMH6 | | 0.344 | 13 | 38 |
| G | O | OH | OH | DMPP | | 6.6 | 563 | 85 |
| J | O | OH | OH | MCPE | | 11 | 659 | 60 |
| L | O | OH | OH | DMP | | 3.8 | 446 | 117 |
| M | O | OH | OH | EMP | | 40.8 | 3900 | 96 |
| N | O | OH | OH | DMBP | | 0.36 | 50 | 139 |
| P | O | OH | OH | DMCP | | 1.55 | 227 | 146 |
| Q | | OH | OH | DMEP | CH₃ | 12 | 640 | 53 |
| R | O | Succinate | OH | DMH | | 1.2 | 41 | 34 |
| S | O | Succinate | Succinate | DMH | | 1.52 | 117 | 77 |
| Y | O | Succinate | OH | DMP | | 7.4 | 315 | 42 |
| Z | O | Fumarate | OH | DMH | | 1.2 | 816 | 656 |
| AH | | OH | OH | DMP | CH₂OC(O) | 42 | 398 | 9.5 |
| | | | | | C(CH₃)₃ | | | |

Compounds with an asterisk do not fall in the definitions of formulae (I) and (II) and are included for comparison only. HU-210 was disclosed in U.S. Pat. No. 5,284,867 and HU-308 was disclosed in international patent application WO 01/32169.

### Example 2

### Anti-Inflammatory Properties of the Bicyclic CB2 Ligands in Vitro

Specific aspects of the inflammatory response cascade are mediated by cytokines, such as TNF-α, IFN-γ, IL-2 and IL-1β and by inflammatory mediator such as COX-2 and PGE₂. Modulating the levels of these pro-inflammatory agents is very important for the severity of the final inflammatory outcome. These agents are also produced by activated cells of the immune system, and the purpose of this study is to test the impact of the new bicyclic CB2 ligands on secretion of these inflammatory agents from activated macrophages and T cells. The levels of secretion in the various test groups are measured by ELISA assays and the level of inhibition is calculated versus the vehicle treated group.

Quantitation of Protein Using ELISA.

The technique used to quantify the amount of a given protein in a liquid sample, either tissue culture supernatant or body fluid, is based on Enzyme Linked ImmunoSorbent Assay (ELISA) methodology. Either commercially available or established in house, the assay is based on the capture of the protein of interest by specific antibodies bound to the bottom of an ELISA plate well. Unbound material is washed away, the captured protein is then exposed to a secondary antibody generally labeled with horseradish peroxidase (HRP) or alkaline phosphatase (ALP). Again the unbound material is washed away, the samples are then incubated with the appropriate substrate yielding a calorimetric reaction. The reaction is stopped and reading is performed in a spectrophotometer at the appropriate wavelength. Samples are tested at least in duplicate and the appropriate standard curve, consisting of serial dilutions of the recombinant target protein, is incorporated on each plate. Concentration of the protein in the sample is calculated from the standard curve.

Macrophage Activation.

RAW 264.7 macrophages, a mouse cell line (ATCC # TIB-71), were grown in Dulbecco's modified Eagle's medium (DMEM) with 4 mM L-glutamine adjusted to contain 1.5 g/L sodium bicarbonate, 4.5 g/L glucose, and 10% heat inactivated fetal bovine serum. Cells were grown in tissue culture flasks and seeded at appropriate density into 24 wells tissue culture plates. 0.5×10⁶ Raw cells in one milliliter were stimulated with 2 μg/ml Lipopolysaccharide E. coli 055:B5 (DIFCO Laboratories). The mouse macrophages were pre-treated for one hour with controls or 10 μM of bicyclic CB2 ligands and later on activated with LPS. Dexamethasone was used as a positive control at 50 nM. Supernatant was collected 4 hours (for PGE₂) and 24 hours (for IL-1β and TNF-α) after activation, and the levels of the inflammatory agent under study were determined by ELISA, as previously described. Inhibition was calculated versus vehicle treated cells.

Inhibition of IL-1β in Activated Macrophages.

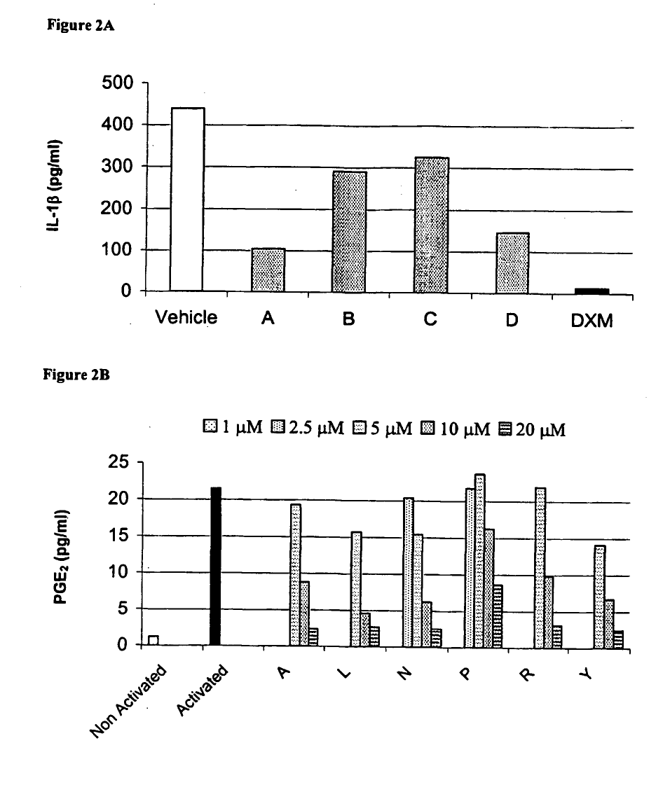

The results obtained for IL-1β are depicted in FIG. 2A where the levels of secretion are plotted for each treatment group. From this figure we can see that bicyclic CB2 ligands can be at 10 μM potent inhibitors of IL-1β, compound A inhibits 76% of the secretion, compound D inhibits 67%, compound B inhibits 34% and compound C inhibits 26%. Dexamethasone inhibited 97% of IL-1β secretion in the same experiment.

Inhibition of TNF-α in Activated Macrophages.

The activation of the macrophages is performed as previously described. The levels of TNF-α are measured in an ELISA assay as previously described. Inhibition is calculated versus vehicle treated cells. Treatment with 10 μM of compound A reduced TNF-α secretion by 53% and IC₅₀ was calculated to be 10 μM. Treatment was performed at various doses of test compounds ranging from 1 μM to 20 μM in order to determine IC₅₀ values of other compounds of the invention, such as compounds L, N, P, R and Y, and none of them did significantly affect TNF-α secretion at doses up to 20 μM.

Inhibition of PGE₂ in Activated Macrophages.

The activation of the macrophages is performed as previously described. The levels of PGE₂ are measured in an ELISA assay as previously described. Inhibition is calculated versus vehicle treated cells. Treatment is performed at various doses of test compounds ranging from 1 μM to 20 μM in order to determine IC₅₀ values. Results are depicted in FIG. 2B. The IC₅₀ values for the inhibition of PGE₂ secretion by compounds A, L, N, P, R, and Y were respectively 9 μM, 7 μM, 7 μM, 18 μM, 9 μM, and 7 μM.

T Cell Activation.

Jurkat cells (human acute lymphoma T-cell line; ATCC # TIB-152) are grown in RPMI 1640 medium with 2 mM L-glutamine adjusted to contain 1.5 g/L sodium bicarbonate, 4.5 g/L glucose, 10 mM HEPES, 1.0 mM sodium pyruvate, and 10% heat inactivated fetal bovine serum. Cells are grown in tissue culture flasks and seeded at appropriate density into 24 wells tissue culture plates. 2×10⁶ cells in one milliliter are stimulated using 10 ng/ml of PMA (Sigma) and 1 μM A23187 calcium ionophore (Sigma). Cyclosporin A (Sandoz), a known immunosuppressive drug, is used as positive control. The controls and test compounds are added at indicated concentrations one hour before stimulation. Supernatant is collected 24 hours after stimulation and the levels of the inflammatory agent under study are measured in an ELISA assay as previously described. Inhibition is calculated versus vehicle treated cells.

Inhibition of 2 in Activated T Cells.

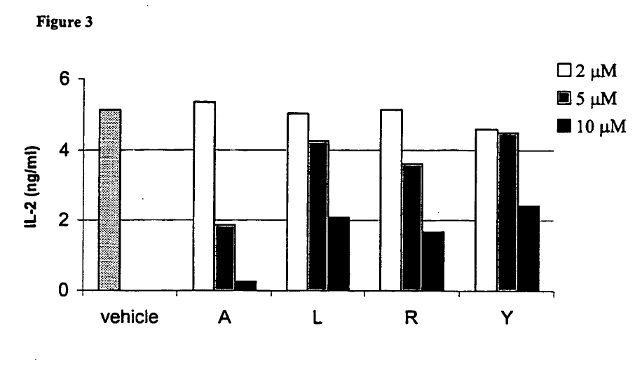

The activation of the T cells is performed as previously described. The levels of IL-2 are measured in an ELISA assay as previously described. Inhibition is calculated versus vehicle treated cells. The results of this experiment are depicted in FIG. 3 were the levels of IL-2 secretion achieved by vehicle or compounds treated cells are plotted for each concentration. From this figure we can see that bicyclic CB2 ligands can be potent inhibitors of IL-2, compound A has a calculated IC₅₀ of 3 μM while compounds L, R and Y have a calculated IC₅₀ of 8 μM, 9 μM and 9 μM, respectively. HU-308, from which the family of bicyclic synthetic cannabinoids has evolved, has itself minimal effect in this experimental setup at doses of up to 10 μM. Cyclosporin A at a concentration of 10 nM inhibited 98% of IL-2 secretion in the same experiment. It should be noted that compounds A and L were also tested in this experimental setup in presence of 0.5-5 μM of the CB1 antagonist SR141716A or of the CB2 antagonist SR144528, and that their IL-2 secretion inhibiting activity was not reversed by any of these antagonists. This observation might be explained either by the fact that the antagonists are not fully adequate to block this specific potentially receptor-mediated activity or by the hypothesis that some of the compounds' activities might not be mediated by CB2 binding but by alternative mechanisms, for instance through binding to additional yet unidentified cannabinoid receptors or through non-receptor mediated mechanisms.

Altogether these experimental results support the conclusion that bicyclic CB2 binding compounds of the invention are potent inhibitors of pro-inflammatory agents secretion from activated cells of the immune system, whether through CB2 binding or through alternative mechanisms.

Mast Cell Activation.

Mast cells are multifunctional bone marrow derived cells that upon activation release potent inflammatory mediators. Release is done either from preformed granules, trough the process of degranulation, or following stimulation-induced de novo synthesis. The molecules released by Mast cells include biogenic amines such as histamine, chemokines, cytokines, enzymes, growth factors, peptides, arachidonic acid products and proteoglycans. It should be noted that mast cells are also known to play a key role in generating pain signal. RBL2H3 cells (rat basophilic leukemia cell line; ATCC # CRL-2256) express a CB2 like receptor and are most appropriate for the study of the mechanisms underlying the anti-inflammatory activity of CB2 selective ligands. RBL-2H3 can be stimulated either by IgE dependent mechanism or by addition of PMA and Calcium ionophore.

RBL-2H3 cells are grown in EMEM medium with Earle's BSS, 2 mM L-glutamine adjusted to contain 1.5 g/L sodium bicarbonate, 0.1 mM non essential amino acids, 1.0 mM sodium pyruvate, and 15% heat inactivated fetal calf serum. Cells are grown in tissue culture flasks and seeded at appropriate density into 24 wells tissue culture plates. 2×10⁵ cells in one milliliter are stimulated by either one of the following. First, the IgE dependent method wherein after overnight plating, medium is replaced and cells are sensitized for one hour with medium containing 0.5 μg/ml anti-DNP (dinitrophenyl) conjugated to IgE (Sigma, Cat. No. D-8406). Cells are then washed twice with PBS and exposed to new pre-warmed medium containing 0.1 μg/ml DNP-HAS (dinitrophenyl albumin, human serum, Sigma, Cat No. A-6661). Test compounds and controls, diluted in DMSO, are added before the ultimate stimulus at final concentration not exceeding 0.1% DMSO. The degranulation process is allowed to proceed at 37° C. for various periods of time, depending on the mediator to be assessed, and 200 μl of supernatant are then collected. For instance cells are stimulated for 1 hour before sampling for histamine and for three hours for monitoring serotonin, TNF-α and IL-4 levels of secretion. The second possibility to use this model is when the stimulation is achieved using 10 ng/ml of PMA (Sigma) and 1 μM A23187 calcium ionophore (Sigma). The Src family inhibitor PP1 or the PKC inhibitor GF109203X (both from Calbiochem) are used as positive control. The controls and test compounds are added at indicated concentrations before stimulation. Supernatant is collected up to 24 hours after stimulation, depending upon the mediator under study, and the levels of this agent are measured in an ELISA assay as previously described. Inhibition is calculated versus vehicle treated cells.

### Example 3

### Effect of Compounds on Gene Expression

The inhibitory activity displayed by some bicyclic CB2 binding compounds on the secretion of inflammatory agents in activated cells of the immune system, either in vitro or in vivo, may be related to regulation of gene expression.

RNA Preparation and Real-Time RT-PCR.

Total RNA is prepared using SV total RNA isolation system (Promega). The cells or tissues are homogenized in lysis buffer. The lysates are transferred to an RNA isolation column, treated with DNAse, washed and eluted according to kit instructions. RNA concentrations were determined using GeneQuant II (Pharmacia-Amersham). Complementary DNA (cDNA) is synthesized from total RNA using SUPERSCRIPT II reverse transcriptase (Life Technologies). 2 μg of total RNA are combined with an oligo (dT)₁₅ primer, 0.5 mM dNTP mix, 8 units of reverse transcriptase and other reaction components up to a final volume of 20 μl, according to the kit instructions. The reaction mixture is incubated at 42° C. for 45 min and inactivated at 70° C. for 15 minutes. Quantitative real-time RT-PCR includes 1 μl of the cDNA, 300 nM of the appropriate forward and reverse primers (according to the gene monitored) and 7.5 μl of the reaction mix containing buffer, nucleotides, Taq polymerase and SYBER green (SYBER Green master mix, Applied Biosystems), in a total reaction volume of 15 μl. Gene amplification is obtained using the GeneAmp 5700 sequence detection system (Applied Biosystems). Amplification includes one stage of 10 minutes at 95° C. followed by 40 cycles of a 2-steps loop: 20 seconds at 95° C., and 1 minute at 60° C. During each annealing step, the amount of the amplified product is measured by the fluorescence of the double strand DNA binding dye, SYBER Green. The cycle of threshold (C_{T}), representing the PCR cycle at which an increase in fluorescence above a baseline signal can be first detected, is determined for each product. A delay of one PCR cycle in the C_{T} is translated into a two-fold decrease in starting template molecules and vice versa. The changes in the C_{T} of the specific gene product are normalized to the changes in the C_{T} of a reference gene cyclophilin or GAPDH. Results are expressed as fold increase of gene expression in the test system above the appropriate control, such as inactivated cell lines or vehicle “treated” animals. In all cases, results are also normalized to a reference house-keeping gene, such as cyclophilin or GAPDH.

### Example 4

### Effect of Compounds in ConA Induced Liver Injury

The hepatoprotective activity of the bicyclic CB2 binding compounds was assessed in the concanavalin A induced liver injury murine model.

The ConA Model for T-Cell Mediated Injury.

The most common causes of life threatening T-cell mediated liver damage in humans are infections with hepatitis B or C viruses and autoimmune hepatitis. Different animal models of autoimmune liver injury have been developed, including acute liver failure in mice induced by intravenous injection of the T-cell stimulatory plant lectin concanavalin A (ConA). ConA has high affinity for the hepatic sinus. Treatment of mice with ConA activates T-cells that accumulate in the liver and release cytokines (such as IL-6, IL-10, TNF-α, INF-γ, IL-2) that regulate liver damage. Pretreatment with the immunosuppressor drugs such as cyclosporin A or FK506 completely prevents liver injury caused by ConA injection, demonstrating the major role of T-cell activation in this model.

Each experimental group contains at least 5 BALB/c inbred female mice (25 g average weight, Harlan, Israel). The negative control group is composed of mice injected with saline instead of ConA. The injection of ConA (Sigma) is done i.v. at the base of the tail at the dose of 10 mg/kg in saline. The treatments are injected i.v. at 1 mg/kg, 30 minutes prior to the ConA injection. Compounds are dissolved in CREMOPHOR EL®:ethanol and vehicle only was included as an internal control.

Impact of treatment is monitored at three levels. First, blood samples (200-400 μl) are collected at predetermined time points alter ConA injection, using retro-orbital puncture. After short centrifugation (5000 rpm for 2 min) serum is recovered and stored at −80° C. until further use for determination of cytokines levels by ELISA and aminotransferase leakage from the liver as a marker for liver injury. In parallel, the level of cytokines, or other inflammatory mediators, is also determined in the organs of interests. For this purpose, the mice are killed by dislocation of the cervical vertebrae, at predetermined time points following ConA injection. The spleen and the liver are removed. Part of the liver is fixed in 4% formaldehyde and the other part was kept at −80° C. for protein or RNA extraction. The spleens are weighted and a small part of the spleen is fixed in 4% formaldehyde, while most of the organ is cultured according to the following procedure. Each spleen is squeezed through a cell strainer with the rough end of a 5 ml syringe into 4 ml of RPMI medium. Large tissue fragments are removed by gravity sedimentation and the supernatants are collected. Cells are washed 3 times with 5 ml of erythrocyte lysis buffer (Boehringer), resuspended in 4 ml RPMI medium supplemented with 2 mM L-glutamine adjusted to contain 1.5 g/L sodium bicarbonate, 4.5 g/L glucose, 10 mM HEPES, 1.0 mM sodium pyruvate, and 10% heat inactivated fetal bovine serum, and plated in a 6 wells culture dish. Cells are incubated for 24 hours and cytokine levels in the supernatant are determined by ELISA as previously described.

The effect of compound A on liver injury was assessed by measuring the level of ALT in the plasma following 8 hours of ConA treatment. Exposure to ConA caused a dramatic increase in ALT plasma concentrations from 30 to more than 2800 IU/l. Animals treated with vehicle only showed a non-significant reduction of 29% in ALT while animals treated with 1 mg/kg of compound A displayed a significant 65% decrease. ALT being an established marker of liver injury, these experimental results support the potential therapeutic effect of bicyclic CB2 ligands in liver inflammation.

### Example 5

### Effect of Compounds in Brain Tissue Following LPS Injection

The neuroprotective activity of bicyclic CB2 binding compounds in cases of CNS inflammation is assessed in vivo in a model wherein the inflammatory injury is generated by injecting LPS into the mice cerebral ventricules. PBS is used as control. LPS is dissolved in PBS at 50 ng/μl and 5 μl are injected in each ventricule at a rate of 1 μl/min with the help of a syringe pump and a brain infusion cannula After each injection, the cannula is left in situ for one more minute to avoid reflux. The various treatment groups and controls are injected i.p. (0.1 ml/10 g body weight) immediately after i.c.v (intra cerebral ventricular) injection of LPS. Each treatment group is composed of five C57/BL male mice (6-8 weeks old, 25 g average weight, Harlan, Israel). Six hours following LPS injection, the animals are sacrificed by i.p. injection of 100 mg/kg pentobarbitone sodium and their brains are removed and kept at −80° C. until next step. RNA is extracted from each whole brain and gene expression levels of inflammatory agents are analyzed by real-time RT-PCR as previously described. The results of this experiment are expressed as fold activation of gene under study in LPS versus PBS injected brains.

This experimental model also allows monitoring the effect of bicyclic CB2 binding compounds on cerebral inflammation by measuring the extent of gliosis. For this purpose the animals are sacrificed 3 days following LPS and treatment injection and their brains are removed. Frozen sections of 20 μm are cut at the level of the hippocampus and stained by standard immunohistochemistry method using antibodies against the F4/80 marker. Quantitative analysis is performed by counting the F4/80 immunoreactive cells. The differences between the treatment groups are compared using analysis of variance ANOVA followed by post-hoc t-Test. A value of p<0.05 is considered to be statistically significant.

### Example 6

### Effect of Compounds in Middle Cerebral Artery Occlusion

Transient MCAo in Mice

The neuroprotective activity of the compounds of the invention is assessed in the middle cerebral artery occlusion (MCAo) murine model, mimicking cerebral ischemia. This model corresponds to cerebral ischemia as observed in stroke. Mice (C57/B1, male, 25 g average body weight, Harlan, Israel) are anaesthetized with halothane in 30% oxygen and 70% nitrogen (4% for induction in an anesthesia chamber, and 1-2% in a facemask for maintenance). A midline incision is made in the skin of the neck, and the tissue underneath is bluntly dissected. The right common carotid artery (CCA) and its junction with the external carotid artery (ECA) and internal carotid artery (ICA) are explored by blunt dissection. The branches of the ECA, the occipital and the superior thyroid artery, are then cauterized. The CCA is then transiently closed by positioning around it a 5-0 silk suture material (Assut, Switzerland). Two cm. pieces of the nylon suture material are cut and placed in a solution of 1% Poly-L-Lysine and then dried in an oven (60° C.) for 60 minutes. The tip of each piece is rounded under a flame. The ECA is permanently occluded with the same type of suture material. A third closure, transient this time, is done in the ICA with 5-0 silk suture material. A small hole is cut in the ECA and the nylon thread is inserted into the ICA while avoiding entrance into the pterygopalatine artery. The thread is inserted 11 mm until a slight resistance is felt. Then a 5-0 silk suture knot secures the thread. One cm of the thread left outside are then cut. The skin wound is closed by 5-0 silk suture material.

Following the operation, the animals are allowed to wake up in the cage. One-hour post insult initiation animals are clinically tested to verify the success of MCA occlusion. The evaluating system was based on works by Belayev et al., (Stroke 27: 1616-23, 1996; Brain Res. 833: 181-90, 1999). It consists of two tests: the postural reflex test and the fore limb-placing test. The postural reflex is evaluated while the animal is suspended by the tail, while the fore limb-placing test is performed while the animal is held by the stomach. Table 1 summarizes the tests and their scoring system.

TABLE 1
Neurological evaluation of mice with MCAo.

| | Normal | |
|---|---|---|
| Item | Score | Deficit |
| Postural reflex test (hang test)* | 0 | 2 |
| Placing test (performed on each side)# | | |
| Visual placing | | |
| Forward | 0 | 2 |
| Sideways | 0 | 2 |
| Tactile placing | | |
| Dorsal surface of paw | 0 | 2 |
| Lateral surface of paw | 0 | 2 |
| Proprioceptive placing | 0 | 2 |

*Scores are as follows: 0 no observable deficit, 1 limb flexion during hang test, 2 deficit on lateral push.
#Scores are as follows: 0 complete immediate placing, 1 incomplete or delayed placing (>2 seconds), 2 absence of placing.

Only animals with total scores between 8 to 12 are included in the study. Ninety minutes after initiation of the insult, the selected animals are resedated using the same method, the neck wound is then re-opened and the nylon thread is pulled out of the ICA. The skin wound is then closed with 5-0 silk suture material. The controls and test compounds are administered 1 minute before the end of the insult. All treatments are delivered i.v. 5 mg/kg. Vehicle is administered 5 ml/kg. Each treatment group comprises at least 6 animals. The animals are then followed up for three main parameters: a clinical functional evaluation, a histopathological evaluation including extent of insult and an assessment of immune/inflammatory markers. At the end of the study, animals are sacrificed by i.p. injection of pentobarbitone sodium 100 mg/kg. Brains are then removed and prepared for examination. Total RNA is prepared from the ipsilateral half of the brains for monitoring the impact of test compounds on markers of ischemia. Gene expression levels are analyzed by real-time RT-PCR as previously described. Results are expressed as fold activation over sham operated animals. Gene expression is normalized to house-keeping gene cyclophilin.

### Example 7

### Treatment of Inflammation: the Ear Edema Model in the Mouse

The anti-inflammatory activity of the novel bicyclic CB2 ligands was screened in vivo using an ear edema model in mice. This test system utilizes various inflammation inducers, including Croton oil (CO) and Arachidonic acid (AA) and the outcome is assessed by measuring ear tissue swelling. Nonsteroidal anti-inflammatory drugs have been shown to reduce swelling in this model (Young, J. M. et al., J. Invest. Dermatol. 82: 367-71, 1984). The ability of the test compounds to prevent or diminish the inflammatory response to these stimulants is indicative of their systemic anti-inflammatory capability.

Compound A was dissolved in CREMOPHOR EL®:ethanol and injected i.p. in adult male ICR mice (30 g average body weight, Harlan, Israel) after dilution with sterile 0.9% sodium chloride to desired final concentrations according to required doses. Various doses of compounds were checked ranging from 0 to 30 mg/kg. Each treatment group was composed of 8-10 animals while the vehicle treated group was composed of 16 animals. Inflammation was immediately induced by applying 20 μl of 50% CO in acetone to the outer surface of one ear, the contralateral ear was exposed to acetone only and served as control. Ear thickness was determined (in 0.01 mm units) 3 hours after CO application using a dial thickness gauge (Mitutoyo, Japan). Finally the ears were trimmed, an ear punch of 6 mm diameter was removed and its weight was measured. The ear edema is expressed as the ratio of ear punch weight of the CO treated ear versus the contralateral Acetone treated ear. Results are calculated as % inhibition as compared to CREMOPHOR EL®:ethanol vehicle treated animals. From the analysis of the dose response performed in this study we see that compound A has an ED₅₀ of 30 mg/kg or 81 μmole/kg when injected intraperitoneally. These results show that bicyclic CB2 ligands can function as systemic anti-inflammatory compounds.

### Example 8

### Treatment of Inflammation: the Paw Edema Model in the Mouse

The purpose of this study is to test in vivo the anti-inflammatory activity of the compounds in paw edema induced by injection of 1% carrageenan in the animal hind paw. Female Balb/c mice (20 g average body weight, Harlan, Israel) are anesthetized with a combination of xylazine and pentobarbitone diluted in sterile saline, 15 and 6 mg/kg i.p. respectively. Anesthetized mice are injected subcutaneously, in the subplantar region of one (right) paw with 0.05 ml of 1% w/v Carrageenan in sterile water. The contralateral (left) paw is not injected as data from the literature, confirmed by our own experience, showed that injection of 0.05 ml of normal saline did not affect later thickness or volume measurements. The test compounds, including known anti-inflammatory controls, are dissolved in CREMOPHOR EL®:ethanol and further diluted 1:20 or 1:50 in sterile saline prior to i.p. injection that takes place immediately before the carrageenan injection. Three hours after injection the animals are resedated following the previously described procedure. Paw thickness is measured using a dial thickness gauge (Spring-dial, constant low pressure gauge, Mitutoyo, TG/L-1, 0.01 mm) and paw volume is measured using a plethysmometer (model #7150, Ugo Basile, Italy). Paw Edema is expressed as the difference between the right treated and the left untreated paws of the same animal, either as Δ Paw Volume (ΔPV) in millimeters cube or as Δ Paw Thickness (ΔPT) in millimeters. Each group comprises at least 10 animals. Results can be further normalized to the ΔPV and ΔPT values of each treatment group at 0 mg/kg (vehicle only). At the end of the study, animals are euthanized with an i.p. injection of 100 mg/kg pentobarbitone.

The results are first calculated as ΔPV or ΔPT, and then further analyzed as % inhibition by comparing the effect of treatment versus vehicle on paw volume or thickness. The differences among various treatment groups are analyzed by analysis of variance (ANOVA) followed by post-hoc Fisher test. A value of p<0.05 is considered to be statistically significant.

When results are expressed as % inhibition of paw thickness, normalized to vehicle, and plotted against the dose of the test compound the resulting pattern is an initial slope up to a maximal observed effect (MOE) at a given dose followed by a plateau at higher doses. Analysis of the anti-inflammatory activity of the test compounds was performed on two parameters, the maximal % inhibition in paw thickness and the dose at which the maximal effect was observed. The first general observation is that the bicyclic CB2 ligands were efficient at low doses comparable to known anti-inflammatory compounds such as Dexamethasone and Celecoxib, all in the range of up to 2 mg/kg. HU-308, the prototype of the bicyclic CB2 ligands, yielded a maximal reduction of about 28% in paw thickness at 0.6 mg/kg. Compound A yielded similar 28% reduction in paw thickness at 2.5 mg/kg, while compounds L and R showed respectively MOE of 34% and 31% at 0.25 and 0.5 mg/kg. For sake of comparison, known anti-inflammatory drugs such as Celecoxib and Dexamethasone yield respectively in the range of relevant doses 7% and 26% reduction in paw thickness at 0.1 mg/kg, 16% and 31% at 0.25 mg/kg and 24% and 33% at 0.5 mg/kg. Thus, most of the compounds tested are at least superior to Celecoxib. It should be kept in mind that these commercially available drugs display serious side effects that prevent chronic uses without complementary protective medication. The fact that compounds of the invention have anti-inflammatory activity comparable to these drugs is very encouraging since compounds of this family have the advantage of being devoid of side effects, thus making them interesting candidates for the replacement of existing anti-inflammatory drugs. These results support that bicyclic CB2 ligands of the present invention have an anti-inflammatory effect that might be relevant to a wide range of human conditions with inflammatory components.

### Example 9

### Experimental Autoimmune Diseases: CIA, EAE and DTH

Autoimmune diseases are associated with elevated levels of inflammatory cytokines. The rodent models most commonly studied are experimental allergic encephalomyelitis (EAE), a model for multiple sclerosis in the human, experimental autoimmune arthritis, a model for rheumatoid arthritis in the human and delayed type hypersensitivity (DTH), a model for allergic reactions in the human. EAE is an autoimmune neurological disease elicited by sensitization of the animals to myelin basic protein from the central nervous system, which is also known as basic encephalitogenic protein. Experimental autoimmune arthritis is induced in animals by immunization with collagen in complete Freund's adjuvant: the model is therefore named collagen induced arthritis (CIA). Delayed type hypersensitivity is induced by the application of dinitrofluorobenzene according to a strict time-schedule, therefore the model generated correspond to allergic contact dermatitis in the human. The purpose of the present study is to test the ability of our compounds to prevent or attenuate the clinical signs of these three autoimmune disease models.

Collagen Induced Arthritis.

Adult DBA/1 male mice (20 g average body weight, Harlan, Israel), at least eight per treatment group are used in this study. Bovine collagen type 2 is dissolved in 0.05 M acetic acid at a concentration of 2 mg/ml by stirring ON at 4° C. The collagen solution is further emulsified in an equal volume of Complete Freund's Adjuvant (CFA). Each animal is administered with 100 μg collagen type 2 in 0.1 ml CFA emulsion. The collagen is administered s.c. at the base of the tail. Twenty-one day after priming, the mice receive an intradermal booster injection of 100 μg collagen in Incomplete Freund's adjuvant.

The volume of each hind paw is measured using a plethysmometer (Hugo Basill, Italy), and the thickness using a dial, constant pressure gauge, (Mitutoyo, Japan). Measurements are performed before collagen administration and every second day throughout the designated follow-up period. All treatments are administered intraperitoneally. At the end of the treatment period the animals are sacrificed with pentobarbital 100 mg/kg i.p.

The differences between the severity of the paw swelling among various treatment groups are compared using analysis of variance ANOVA followed by post-hoc t-Test. A value of p<0.05 is considered to be statistically significant.

Experimental Autoimmune Encephalomyelitis.

Various animal models of autoimmune encephalomyelitis are known in the art, depending on the method of induction, the strain of the animal and the antigen employed to induce the disease. The impact of bicyclic CB2 ligands was tested in EAE using Lewis rats in which the onset of disease is observed by the appearance of clinical symptoms about 10 days after induction. The disease progress and the clinical score increase and peak around day 15 and spontaneous recovery is observed around day 18 after induction of the disease. The animals (at least 9 per test group at initiation of study, except for the untreated control group that comprised only 5 rats) were maintained on a 12 hours light/12 hours dark regimen, at a constant temperature of 22° C., with food and water ad libitum. EAE was induced in these animals by immunization with s.c. injection to the hind paws of 25 μg of purified guinea pig myelin basic protein (MBP, Sigma) emulsified in 0.1 ml of Complete Freund's Adjuvant (Difco).

Animals that exhibited symptom of the disease, which could be clinically scored between 0.5 and 1, were treated with test compounds or vehicle control, administered intravenously in a volume of 5 ml/kg, for three consecutive days starting from the onset of the disease (˜ at day 10 following disease induction). Methylprednisolone was used as positive control and it was administered daily for 5 consecutive days i.v. at 30 mg/kg starting from day of disease induction by MBP injection. The results are recorded as clinical score; score of 0 indicates a normal animal with no clinical signs, 0.5 indicates a loss of tonicity in the tail's distal part, 1 indicates whole tail paralysis, 2 indicates paraplegia, 3 indicates quadriplegia, 4 indicates complete body paralysis and moribund state and 5 indicates death. The clinical score of the animals is recorded for 11 days following onset of disease and the area under the curve (AUC) is calculated over this period of time. The differences between the severity of the clinical outcomes among various treatment groups was analyzed by analysis of variance (ANOVA) followed by Fisher's LSD test. A value of p<0.05 is considered to be statistically significant.

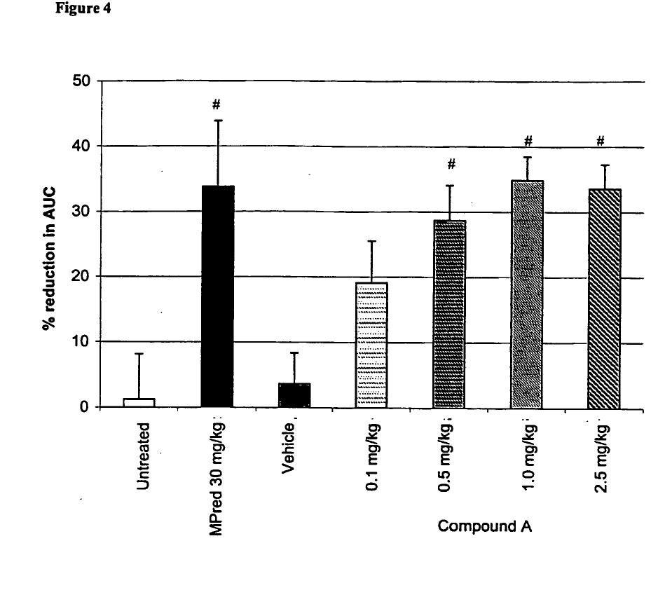

Results are displayed in FIG. 4 as the % of reduction in the average AUC for each treatment group. Compound A yielded a reduction in the AUC of the clinical score in a dose related manner, with a significant reduction of 35% at the dose of 1 mg/kg. Results statistically better (p<0.05) than the results obtained with untreated and CREMOPHOR EL®:ethanol vehicle treated animals are indicated by a # in FIG. 4. In this experimental setup, the positive control methylprednisolone (MPred) yielded 34% reduction when administered 5 times before the disease onset at the dose of 30 mg/kg. Benzyl alcohol served as MPred's vehicle and by itself increased the AUC by 18%, data not shown in figure. This experiment was independently repeated in a blinded manner and similar results were obtained, with for example 30% reduction in the clinical score with 1 mg/kg of compound A.

Moreover, in a separate study animals were euthanized 15 days after induction of the disease by i.p. injection of 100 mg/kg pentobarbital. Brains and spinal cord were removed and were fixed by overnight incubation with 4% Paraformaldehyde. The cervical segment of the spinal cord was dehydrated using ethanol solutions of increasing concentration and then embedded in paraplast. The spinal cord was then sectioned (10 μm) and staining was performed using hematoxylin and eosin. The stained slides were examined under light microscopy for foci of infiltrating lymphocytes. Number of foci were counted and averaged in 6 sections for each animal. Three groups of at least 4 animals each were tested in this system: untreated, vehicle treated, and animals treated with 0.5 mg/kg of compound A. Results are expressed as average±SD of number of foci. The differences between the number of infiltration foci among various treatment groups was analyzed by analysis of variance (ANOVA) followed by Student t test. A value of p<0.05 is considered to be statistically significant.

Untreated animals displayed the highest number of infiltration foci in their spinal cords with an average of 23±16 foci/section. Treatment with vehicle only had no effect on this outcome with 21±6 foci, whereas 0.5 mg.kg of compound A significantly decreased infiltration by more than 50% with 10±5 foci/section. These observations were made when the disease is already established (˜5 days since onset) and support the fact that bicyclic CB2 ligands are potent neuroprotector by prevention of infiltration of cells that yield deleterious inflammatory/immune cascades.

Altogether, these experimental results suggest that bicyclic CB2 ligands are effective treatments in model relevant to human multiple sclerosis, both at the histological level in the nervous system and at the level of the functional clinical outcome.

Delayed Type Hypersensitivity in Mice.

Adult female BALB/c mice (20 g average body weight, Harlan, Israel) were sensitized on day 0 and day 1 by application of 30 μl of 0.15% Dinitrofluorobenzene (DNFB) diluted in acetone on the shaved skin of the abdomen. On day 6 the animals were challenged by application of 10 μl of DNFB in acetone on one ear. The contralateral ear was not challenged but received the application of 10 μl acetone. Test compounds were administered at increasing doses from 0 to 15 mg/kg i.p. twice, the first injection was immediately after DNFB challenge (on day 6) and the second injection was 16 hours post challenge (on day 7). Each treatment group comprised at least 7 animals. Dexamethasone (DXM) was used as positive control. Ear thickness was determined (in 0.01 mm units) 24 hours after challenge (and 6 hours after second treatment on day 7) using a dial thickness gauge (Mitutoyo, Japan).

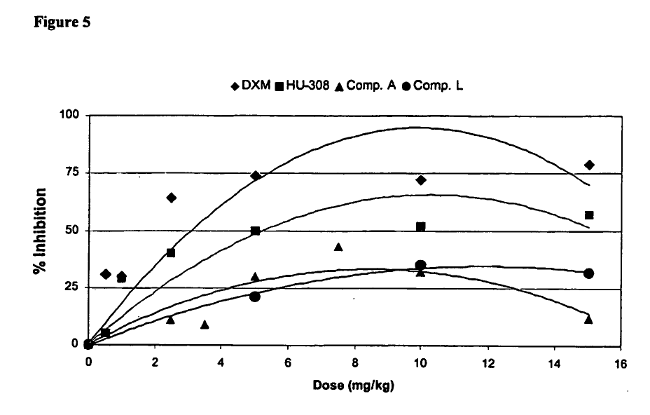

Results are analyzed as ear thickness of DNFB treated over DNFB untreated contralateral ear. The impact of the test compound was further assessed by comparing its mean impact on the animals of the treatment group to the response generated by the appropriate vehicle only. Results are displayed in FIG. 5 where % of reduction in ear thickness is plotted against the treatment dose. Generally speaking the pattern obtained is that of a curve reaching a plateau of activity. For the positive control we can see that the maximal inhibition is around 80% while for HU-308 the maximal inhibition is in the range of 60%. The calculated IC₅₀ are 3.2 mg/kg for dexamethasone and 4.8 mg/kg for HU-308. Compounds A and L do not reach 50% inhibition at the doses tested and their maximal reduction is in the range of 35-43%. These experimental results suggest that bicyclic CB2 ligands are effective treatments in model relevant to human allergies and immune responses.

### Example 10

### Treatment of Neurodegenerative Disorders: the MPTP Model

Parkinson's disease (PD) is a neurodegenerative disorder characterized by tremor, slowness of movements, stiffness and poor balance. Most, if not all, of these disabilities are due to a profound reduction in striatal dopamine content caused by loss of dopaminergic neurons in the Substantia Nigra pars compacta (SNpc) and of their projecting nerve fibers in the striatum. 1-Methyl-4-phenyl-1,2,3,6-tetrahydropyridine (MPTP) is a well known neurotoxin that can cause depletion of dopamine content in the striatum and a reduction in the number of nigrostriatal dopaminergic neurons in several species including humans (Turski L. et al., Nature 349: 573, 1991). The aim of the present study was to examine the effect of bicyclic CB2 ligands on the progression of MPTP-induced dopaminergic toxicity.

Animal Treatment and Procedure.

The mice (C57/BL male mice, average weight 30 g, Harlan, Israel) were administered i.p. with 4 injections of MPTP (Sigma, USA) (20 mg/kg, 5 ml/kg) in saline (Teva Medical Israel) at 2 hours interval on day 1. The treatment groups, including: (a) Saline, untreated, (b) MPTP, untreated, (c) Vehicle (1:20of CREMOPHOR EL®:ethanol), 5 ml/kg i.p., and (d) test compounds, were administered i.p. once just before the first MPTP administration. Seven days following the MPTP treatment the animals were sacrificed (by i.p. administration of pentobarbitone sodium CTS Israel 100 mg/kg) and their brains removed for tyrosine hydroxylase (TH) detection using immunohistochemistry technique.

Immunohistochemistry.

Brains were fixed by cardiac perfusion with 4% Paraformaldehyde followed by immersion of the brain in the same fixative for at least 72 hours. Then brains were washed with PBS and transferred to 30% sucrose in PBS until they sank. After the brains sink in the sucrose, they were frozen using the cryostat special fast freezing (−60° C.). The brains were then cryosectioned (20 μm) at the level of the Substantia Nigra (SN). Immunohistochemistry staining was performed using rabbit anti-tyrosine hydroxylase (1:100, Calbiochem). The slides were stained using diaminobenzydine (DAB) detection kit of automated immunostaining system (Ventana). Quantitative analysis was performed by counting of immunoreactive (IR) cells at the widest dimension of the SNpc lateral to the roots of the third cranial nerve separating medial and lateral SN at the level of interpreduncular nucleus. The amount of the labeling at the striatum level was evaluated using computerized image analysis system.

All data are expressed as mean±SD. Data were analyzed using analysis of variance (ANOVA) followed by post-hoc Fisher test. A value of p<0.05 is considered to be statistically significant.

TH Immunoreactivity at the Level of the SNpc.

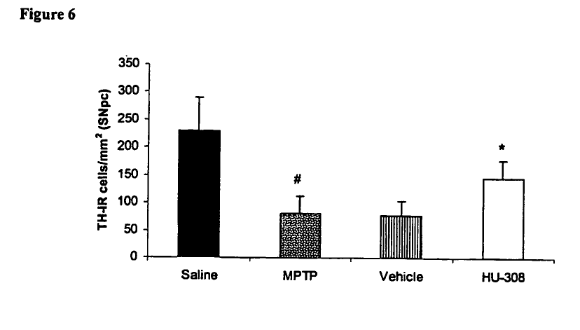

FIG. 6 shows the effect of HU-308, 20 mg/kg i.p., in the MPTP model for Parkinson's disease. The number of TH-IR cells/mm² at the level of the SNpc following MPTP injection and treatment is plotted for each treatment group. Black column—saline injected untreated group. Black dotted column—MPTP injected untreated group. Hatched column—MPTP injected treated with compound vehicle. White column—MPTP injected group treated with HU-308. The signs above the columns refer to the statistical analysis: # p<0.05 compared to saline; *p<0.05 compared to vehicle. Following MPTP injection the number of TH-IR cells decreased by 65% comparing to the saline treated animals (58±10 saline group vs. 20±3 MPTP group). Calculating the TH-IR results from the treated groups relative to the MPTP treated group revealed that HU-308 rescued about 43% of SN dopaminergic cells from MPTP toxicity. The vehicle had by itself no rescue effect on TH-IR cells. These results show that bicyclic CB2 ligands, are effective in models for chronic neurodegenerative diseases such as Parkinson's disease.

### Example 11

### Treatment of Visceral Pain: Attenuation of Mechanical Allodynia

The aim of this study was to assess the potential analgesic effects of the novel bicyclic CB2 binding compounds in an animal model of visceral pain. Visceral pain is caused by disorders of internal organs such as the stomach, kidney, gallbladder, urinary bladder, intestines and others. These disorders include distention from impaction or tumors, ischemia, inflammation and traction on the mesentery, which can cause associated symptoms such as fever, malaise and pain (Al-Haer E. D. et al., Pain 96: 221-5, 2002). The visceral pain was induced in mice by injecting i.p. acetic acid.

Male ICR mice (25 g average body weight, Harlan, Israel) were pretreated by i.v. injection at volume dose of 5 ml/kg of vehicle, control and test compounds at various doses. Each treatment group was composed of at least 4 animals. Fifteen minutes later, the mice were injected i.p. with 10 ml/kg of 0.6% acetic acid and the number of withers is counted over a period of 5 minutes, starting 5 minutes after the acetic acid administration. The results are expressed as mean number of withers±SD. Data were analyzed using analysis of variance (ANOVA) followed by post-hoc Fisher test. A value of p<0.05 is considered to be statistically significant.

Untreated animals displayed on average 30.5±4.3 withers and vehicle only has a slight non-significant effect, reducing the number of withers to 21.8±3.4. However, compound A at doses ranging from 0.5 to 2 mk/kg is highly efficient in this model with statistical significance even at the lowest dose (p=0.0 15). At 0.5 mg/kg, compound A already reduce the number of withers as compared to vehicle by 64% down to 7.9±4.8, at 1 mg/kg the inhibition is as high as 95% with only 1.2±0.6 withers while at 2 mg/kg compound A display full protection with 100% inhibition and no withers at all. These experimental results support that bicyclic CB2 binding compounds are potent analgesic and are protective against visceral pain.

### Example 12

### Treatment of Chronic Neuropathic Pain: Attenuation of Mechanical Allodynia

The aim of this study was to assess the potential analgesic effects of the novel bicyclic CB2 binding compounds in an animal model of neuropathic pain. A peripheral monopathy was induced in the right hind limb of rats following a chronic constriction of the sciatic nerve (Bennet, G. J. & Xie, Y-K., Pain 33: 87-107, 1988). The development of mechanical allodyna was monitored using an established behavioral test (Von Frey filaments).

Pre-surgery baseline values were ascertained as the mean of 2 pre-surgery values. Once the baseline values had been established, the animals were surgically prepared by constricting the right sciatic nerve with 4 chromic cat gut loose ligatures. On day 11 post-operation, the animals that have developed mechanical allodyna were arbitrarily allocated to the various treatment groups based on the pre-surgery values.

The design was randomized, performed in a masked fashion as to whether drug or vehicle is being given. The animals, male Sprague-Dawley rats (average body weight 240-290 g), were allowed to acclimatize to the behavioral testing equipment prior to testing. On the testing day, the animals, at least six per treatment group, were given i.p. a single dose of one of the test compounds in a volume of 2.5 ml/kg. Fifteen minutes later, a series of Von Frey filaments (pre-calibrated prior to testing) were applied to the plantar surface of the hind paw, from below. The filaments were applied in ascending order starting with the weakest force and the withdrawal threshold for both the ipsilateral and contralateral hind paws was evaluated. Each filament was indented on the mid-plantar surface of the foot to the point where it just starts to bend; this is repeated approximately 8-10 times per filament at a frequency of approximately 1 Hz. The withdrawal threshold is defined as being the lowest force of two or more consecutive Von Frey's filaments to elicit a reflex withdrawal response (i.e. a brief paw flick) and is measured in grams.

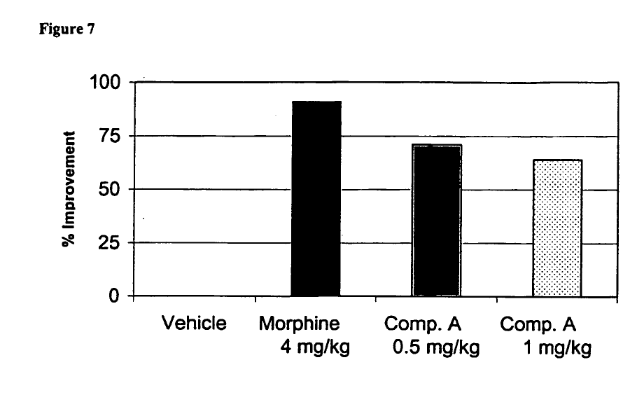

FIG. 7 shows the effect of compound A in the Chronic Constriction Nerve Injury model for Neuropathic pain. The results are expressed as % increase in the threshold of response to Von Frey's filaments in the test compound treated group versus the vehicle treated animals and per definition the vehicle treated group yields a null baseline value. The black column represents the morphine treated animals (4 mg/kg), the gray and the dotted column bars represent two doses of compound A (0.5 and 1 mg/kg respectively). From this study it appears that 15 minutes following treatment, animals treated with Morphine at a dose of 4 mg/kg have a 91% higher pain threshold in their ipsilateral hind paw than those of the vehicle treated group. The groups treated with 0.5 mg/kg and 1 mg/kg of compound A display respectively 71% and 64% improvement in their pain threshold, while in a separate experiment the animals treated with 5 mg/kg of HU-308 show 117% improvement (data not shown). These results teach that bicyclic CB2 ligands of the present invention as well as the known CB2 agonist HU-308 can alleviate or treat chronic neuropathic pain. Thus far, HU-308 was known to alleviate peripheral pain as assessed in the formalin test (WO 01/32169).

Moreover, it should be noted that compound A was found effective in the experimental autoimmune encephalomyelitis system modeling human multiple sclerosis, as described in example 10. Patients suffering from MS not only experience neurological deficits but also develop severe neuropathic pain. The fact that compounds of the invention can tackle simultaneously these two aspects of the disease confer them a clear therapeutical advantage.

### Example 13

### Treatment of Acute Peripheral Pain: the Tail Flick Model

The aim of this study was to assess the potential analgesic effects of the novel bicyclic CB2 binding compounds in an animal model of acute pain. In this model the nociceptive stimulus is thermal and the latency time till the animal flicks its tail is monitored (Le Bars D., Gozariu M. & Cadden S. W., Pharmacol. Rev. 53: 597-652, 2001).

ICR male mice (20-30 g average body weight, Harlan, Israel) were injected i.p. at the volume dose of 5 ml/kg. Each treatment group contained at least 6 animals. Morphine HCl was used as positive control at the final dose of 5 mg/kg. Its vehicle, saline, was also included as control. The test compounds were dissolved in CREMOPHOR EL®:ethanol and diluted 1:20 in saline prior to injection, this second type of vehicle was also included as negative control. The final dose injected varied from 0.1 to 10 mg/kg. At predetermined time points after treatment injection, the animals were placed in the tail flick system (Socrel, model DS 20). Animals were gently held while their tails were located above the photoelectric cell. The tails were then illuminated (21V) at 2 cm from the distal tip and the latency time, measured in seconds, was recorded in duplicates. At the end of the study, the animals were euthanized by i.p. injection of 100 mg/kg sodium pentobarbitone.

Two parameters were used to analyze the results, the latency time and the % of animals showing analgesia. By the later we mean to determine how many animals within a treatment group have increased resistance to pain as measured by a latency time which is superior or equal to twice the latency time observed in vehicle treated animals. The results in both cases are expressed as mean±SE. The differences between the latency times or the % of animals showing analgesia among various treatment groups was analyzed by analysis of variance (ANOVA) followed by post-hoc Tukey's test (for latency) or Fisher's exact test (for % animals). A value of p<0.05 is considered to be statistically significant.

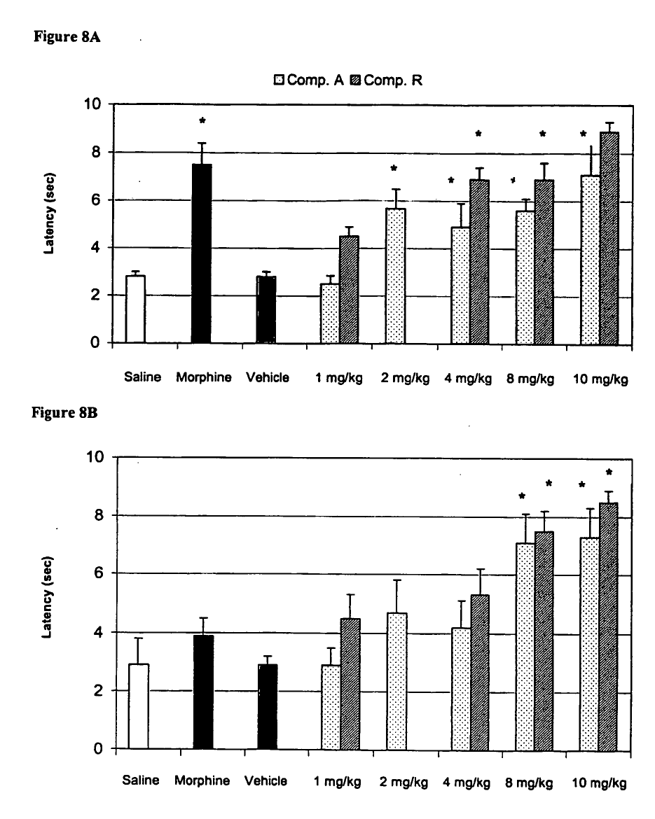

FIG. 8 shows the effect of various doses of compound A (dotted columns) and compound R (hatched columns) in the Tail Flick model for acute pain. When the measurements were performed 30 minutes after injections (panel A), the latency times for the two control groups were similar, 2.65 sec for saline and 2.89 sec for test compounds' vehicle. The positive control morphine increased the latency time to 7.5 sec at 5 mg/kg (p<0.05 as compared to saline, marked by an asterisk on graph). Test compound A significantly (p<0.05 as compared to vehicle, marked by an asterisk on graph) increased the latency time at all doses tested from 2 to 10 mg/kg, with a maximal latency of 7.1 sec at maximal tested dose. When the measurements are performed 90 minutes after injections (panel B), the effect of morphine is significantly reduced and its latency time is now of only 3.9 sec back almost to baseline, whereas the effects of compounds A and R remain relatively stable. At the optimal dose tested, 10 mg/kg, compound A still yielded a latency of 7.3 sec 90 minutes after injection, and compound R maximum latency remained as high as 8.5 sec. The results look even more dramatic when analyzed on the basis of % animals showing analgesia. Then we see that 90 minutes after injection, only about 40% of animals treated with 5 mg/kg morphine still display analgesia whereas more than 80% of animals treated with 10 mg/kg of compound A and 100% of animals treated with 10 mg/kg compound R have latency time twice superior to vehicle treated animals. Significant analgesia was still evident at dosages of 8 and 10 mg/kg of compound A even 330 minutes after injection. Compound R was even more impressive with 100% of animals treated with 10 mg/kg still showing increased analgesia 330 minutes after injection, at this time point the absolute layency time was still as high as 6.8 sec. At lower doses of 4 and 8 mg/kg compound R was still twice better than 5 mg/kg morphine in generating analgesia.

It is interesting to note that the opposite enantiomer of compound A, wherein all other parameters being identical C-5 is R, was tested in this experimental setup and proved to be inefficient. The (+) enantiomer of compound A, namely (4R)-4-[4-(1′,1′-dimethylheptyl)-2,6-dihydroxyphenyl]-6,6-dimethyl-2-norpinanone, was synthetized according to the protocol of Makriyannis and coworkers using as starting material (−)-β-pinene (Drake D. J. et al., J. Med. Chem. 41: 3596-3608, 1998). Animals treated with 4 mg/kg i.p. of compound A, the (−) enantiomer, displayed an increased latency time of 4.9 sec thirty minutes after administration, as compared to 2.8 sec for vehicle treated animals, while animals treated with 4 mg/kg of the (+) enantiomer had an average latency time of only 2.4 sec. The difference between the results obtained with the (+) and (−) enantiomers is statistically significant (two-tails unpaired t test, p=0.04). Moreover, compound A is also more CB2 selective than its (+) enantiomer with an IC₅₀ to CB2 10-fold lower and a CB2/CB1 ratio of about 30, as compared to only 10 for the enantiomer.

Similar studies were performed in Sprague Dawley male rats where the drug was injected i.v. instead of i.p. in the mice model. Comparable results were obtained, the only slight differences concerned the dose necessary to elicit significant increase in latency (lower in i.v. than in i.p.), the onset of action (more rapid in i.v. than in i.p) and the duration of action (shorter in i.v. than in i.p.). These observations are consistent with the route of administration. In the i.p. study the first time point post-injection was 30 minutes, while it was 10 minutes following i.v. injection thus the onset of action was not thoroughly determined in these experiments. Ninety minutes after i.v. injection, 100% of animals treated with either 3 or 4 mg/kg of compound A still had latency time twice superior to vehicle treated animals. At the last time point tested (330 minutes following injection), almost 70% of the animals treated with 4 mg/kg of compound A retained increase resistance to pain.

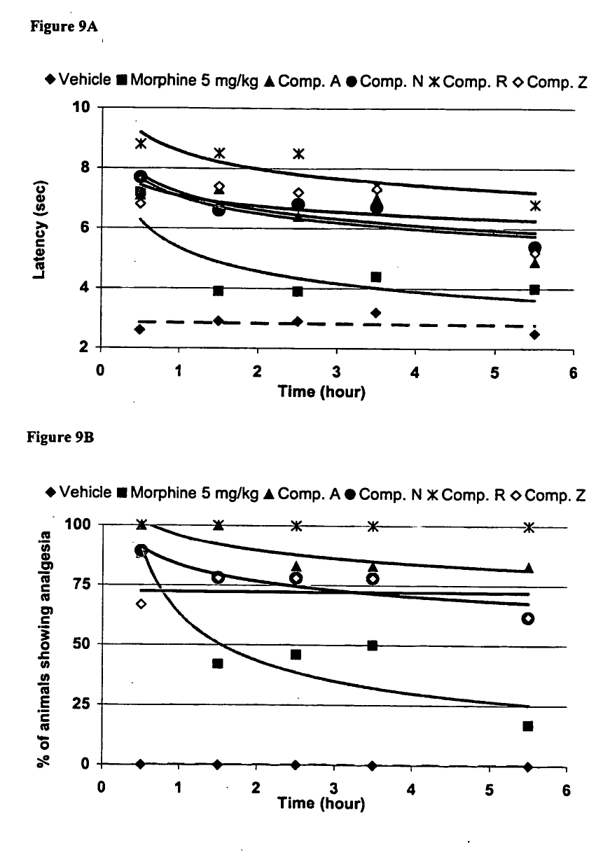

Once an optimal dosage is established, the experiment is repeated at this single dose over longer period of time to establish the duration of the analgesic activity. FIG. 9A shows that at 5 mg/kg of morphine the latency time returns to vehicle baseline values rather rapidly and two and a half hour after injection the latency in morphine treated animals is 3.1 sec as compared to 2.6 sec for the vehicle treated group, previously shown to be similar to saline. These observations are in accordance with the known short-term analgesic activity of morphine. However, compounds A, N, R and Z at the dose of 10 mg/kg generate a sustained analgesic effect till the last time point tested in the experiment. Five and a half hour after injection, compounds A, N, R and Z treated animals show a latency of 4.9, 5.4, 6.8 and 5.2 sec, respectively. At this time point vehicle treated animals have a latency of 2.5 sec until tail flick, while morphine treated animals are slightly protected with a latency of 3.6 sec. FIG. 9B depicts the results of the same experiment when analyzed by the number of animals showing increased analgesia. The results show a similar pattern wherein the number of animals showing increased analgesia rapidly decay in morphine treated animals from 88% half-an hour after injection down to 17% five and an half hours after treatment. The percent of animals displaying improved analgesia decreases at a much more moderate pace in the group treated with 10 mg/kg of compound A, from 100% at initiation of the study down to still 80% five and an half hours later, and from 70-90% for compounds N and Z down to still about 60% at the end of the study. Most impressive results were obtained in the group treated with 10 mg/kg of compound R, wherein 100% of the animals displayed increased resistance to pain, as expressed by a latency time double to vehicle, all along the duration of the study.

Altogether these results teach that bicyclic CB2 ligands have an analgesic effect more prolonged than morphine and they can alleviate or treat acute peripheral pain. While comparable in the early phase of treatment, at most 90 minutes after injection the bicyclic CB2 ligands start to be superior to morphine, both in terms of latency time achieved and in term of % of animals achieving increased latency. It should be beard in mind that compounds of the invention are CB2 selective, but that some do retain physiologically significant binding capacity toward the CB1 receptor as well. We cannot rule out that some of the activities observed are due to CB1 activation alone or in combination with the stronger CB2 activation. Despite the residual CB1 binding activity of some of the compounds of the invention, bicyclic CB2 ligands still have a clear advantage over morphine in the field of side effects, such as tolerance, that will be discussed later.

### Example 14

### Treatment of Inflammatory Pain: the Paw Edema Model in Rats

The purpose of this study is to test the anti-inflammatory pain activity of the compounds in paw edema induced by injection of 2% λ carrageenan in the animal hind paw. Male Sprague Dawley rats (200 g average body weight, Harlan, Israel) are transiently sedated by placement on dry ice for the duration of the injections. Rats are injected subcutaneously, in the subplantar region of one (right) paw with 0.1 ml of 2% w/v λ Carrageenan in sterile saline. The contralateral (left) paw is not injected as data from the literature, confirmed by our own experience, showed that injection of 0.1 ml of normal saline did not affect later analgetic measurements. The test compounds, including known anti-inflammatory controls, are dissolved in CREMOPHOR EL®:ethanol and further diluted 1:20 or 1:50 in sterile saline prior to i.p. injection that takes place immediately after the carrageenan injection. Before induction of inflammatory pain and three hours after injection, the animals reactions to pain stimuli were tested in two systems. The first stimulus was thermal and assessed by the Plantar Test according to Hargreaves, using Ugo Basile Model 7370. The scale was set to an intensity of 50 arbitrary units. The latency time till the animal lift a paw as a reaction to the thermal stimulus was recorded for both the inflamed and non-inflamed hind paws. The second stimulus was mechanical (tactile) and assessed using a Dynamic Plantar Sesthesiomether (Ugo Basile Model 73400-002). The system was set on maximal force of 50 grams and the force applied was gradually increased at the rate of 10 g/sec. At the end of the study, animals are euthanized with an i.p. injection of 100 mg/kg pentobarbitone.

The results are measured as the differences between the two hind paws at time 0 and 3 hours both as ALT, for the latency time in the thermal part of the study, and as ΔForce, for the mechanical part of the study. Results are expressed as mean±SE for each treatment group and the differences among those groups are analyzed by analysis of variance (ANOVA) followed by post-hoc Tukey's test. A value of p<0.05 is considered to be statistically significant.

Administration of 2% λ carrageenan induced paw inflammation, characterized by swelling and redness of the paws. Three hours after inflammation induction, animals untreated or treated with vehicle only displayed a ΔLT of about 5 to 7 seconds between the hind paws following thermal stimulus. This outcome was reduced by about 3-fold when the animals were treated with 8 mg/kg of compound A (ΔLT=1.5 sec) or 10 mg/kg of compound N (ΔLT=2 sec), and down to ΔLT=0 sec when the animals were treated with 10 mg/kg compound R. In this model 5 mg/kg morphine were also effective and reduced ΔLT to 0 second. When the stimulus applied was tactile, it was observed that the force required to cause the rat to lift their paws was reduced by 17 (from 47 g before carrageenan injection down to 30 g three hours later). Again this outcome was very similar in untreated and vehicle treated animals, whereas 8 mg/kg of compound A significantly reduced this outcome down to a ΔForce of only 2 g, compound N yielded a ΔForce of 6 g, compound R yielded a ΔForce of 4 g and compound Z yielded a yielded a ΔForce of only 2 g, the later compounds being tested at 10 mg/kg. At 2 mg/kg compound L caused a reduction in ΔForce from about 20 g in untreated or vehicle treated animals down to 11 g. These values are similar to the results obtained with 1 mg/kg of compound R, which proved to be so potent at higher concentration, however this positive trend bears no statistical significance. In this model 5 mg/kg morphine were also similarly effective and reduced ΔForce to 2.7 grams.

The anti inflammatory pain activity of the bicyclic CB2 ligands was compared not only to an opiate but also to non steroidal anti-inflammatory drugs (NSAID). Three drugs were tested in this model: Celecoxib (COX-2 inhibitor), Ketoprofen (COX-1 inhibitor) and Diclofenac (mixed COX-1 and COX-2 inhibitor). The NSAIDs were tested at three doses: 5, 10 and 20 mg/kg and the intermediate dose of 10 mg/kg was selected for the rest of the study. At 10 mg/kg i.p. all three drugs were very efficient and reduced ΔLT to 0 second, however these results were not significant as opposed to the effect of 10 mg/kg of compound R. When expressed as ΔForce, only Diclofenac and Ketoprofen displayed activity, with respectively 2 and 7 g. These values are in the same range than compounds A, N, R and Z.

It should be noted that compound R was also tested in this experimental setup in presence of 5 mg/kg i.p. of the CB1 antagonist SR141716A or of the CB2 antagonist SR144528, administered 15 minutes before carrageenan and compound. The antagonists by themselves had no analgesic activity. The analgesic activity of compound R against both thermal and mechanical stimuli was not reversed by any of the antagonists. This observation might be explained either by the fact that the antagonists are not fully adequate to block this specific activity or by the hypothesis that some of the compounds' activities might not be mediated by CB2 binding but by alternative mechanisms, for instance through binding to additional yet unidentified cannabinoid receptors or through non-receptor mediated mechanisms.

Altogether these results demonstrate that the bicyclic CB2 ligands of the invention are potent analgesics, with activity comparable or superior to morphine or NSAIDs. Whether this activity is mediated through CB2 binding or through alternative mechanisms remains to be established. The side effects of the commercially available above-mentioned therapeutical agents are well known and the compounds of the invention may advantageously replace them.

### Example 15

### Treatment of Peripheral Noxious Pain: the Formalin Test

Pain mediated by the peripheral nervous system, is tested in the “formalin test” for cutaneous peripheral) pain (Tjolson A. et al, Pain 51: 5-17, 1992). First the test compounds are injected i.p. Then formalin is injected s.c. in the plantar surface of the hind paw of a mouse 90 min after the test compound. Immediately after formalin administration pain is assessed (every 5 min for 1 hr) by the number of times the animal licks the formalin-injected paw.

### Example 16

### Adenylylcyclase Assay

Cannabinoids and derivatives bind to G-protein-coupled CB1 and CB2 receptors and exert their activity via the inhibition of adenylylcyclase activity. An adenylylcyclase assay forms a basis for the functional analysis of the compounds by determining their capacity to inhibit or promote forskolin-activated cAMP production. The assay is carried out according to Chin et al. (Chin, C. N. et al., J. Neurochem. 70: 366-73, 1998). Briefly, HEK-293 human kidney cells (ATCC#CRL-1573) stably transfected with either the human CB1 or CB2 receptor (cDNA) were grown in DMEM supplemented with 10% fetal calf serum, 1% Penicillin-Streptomycin and 2 mM L-glutamine. Cells were seeded in 24 wells plate at 5×10⁴ cells/well and incubated for 48 hours. Medium was then removed and the adherent cells were washed with PBS. Two hundred μl of serum free medium supplemented with 0.2 mM Ro 20-1724, 0.25% BSA, and 20 mM HEPES was then added to each well. The cells were then activated with 1 μM forskolin in presence of the bicyclic test compounds in concentrations ranging from 10 pM up to 1 μM. The activated cells were then incubated at 37° C. for 20 minutes and the reaction terminated with 1.2 M HCl to a final concentration of 0.1 M. Cells were lyzed by freeze and thaw and the lysate was neutralized with 2 M HEPES, pH 7.5. cAMP was measured in 50 μl aliquots using the [³H]-cAMP assay system (Amersham).

Two parameters were assessed in this system, the dose at which 50% of the maximal inhibition of cAMP level is observed (IC₅₀) and the level of inhibition reached with 1 μM of test compound. It must be noted that since forskolin activates cAMP production by many ways a total inhibition of 100% is not to be expected by compounds that should only act on one receptor or on a limited number of pathways of activation. This experiment was first performed on CB2 receptor transfected cells. The IC₅₀ and % inhibition at 1 μM were determined for HU-210 as reference (1.43 nM and 50%), compounds A (0.24 nM and 63%), L (Not Determined and 33%), R (0.47 nM and 64%), S (0.16 nM and 58%) and Y (1.13 nM and 60%). For control, the test compounds were also tested in non-transfected HEK-293 cells and no inhibition could be observed in the levels of cAMP, supporting the fact that the results previously described were indeed specific for the CB2 receptor. The reference compound HU-210 whose IC₅₀ for CB1 binding is 0.328 nM, displayed an IC₅₀ of 0.35 nM for cAMP in CB1 transfected cells, with maximal inhibition at 1 μM of 73%. Similarly, compound A displayed an IC₅₀ of 10.3 nM for inhibition of forskolin induced cAMP production in CB1 transfected cells, with maximal inhibition at 1 μM of 69%. The IC₅₀ for CB1 binding for compound A is in the same range with values of 28 nM. The similitude of IC₅₀ range between binding activity and functional inhibition of cAMP production, further supports the relevance of this experimental setup. These results indicate that the bicyclic CB2 ligands not only bind to the receptor but also elicit the proper functional triggers resulting from adequate receptor activation. From these observations it was deduced that the bicyclic CB2 ligands of the invention act as agonists to the receptor.

In addition, mammalian cells, stably expressing exogenous human CB1 or CB2 receptors and a luciferase reporter gene linked to the cyclic-AMP response element (CRE) are activated with different stimuli, such as forskolin or calcium ionophore. Following activation, the cells are extracted and the activity of the reporter gene is measured in luminescence units by the luciferase assay. Elevation in cyclic-AMP is reflected by an increase in luciferase activity.

### Example 17

### Psychomimetic Effects of the Compounds

Male ICR mice (25 g average body weight, Harlan, Israel) are used for a series of tests for psychotropic effects, specifically locomotor activity and rectal temperature. The test compounds, and known and specific CB1 and CB2 antagonists when appropriate, were dissolved in vehicle, diluted in saline and injected i.p. or i.v. at doses up to 3 mg/kg i.v., in volumes not exceeding 0.05 ml/10 g body weight in mice. Naïve mice were used as control. Each treatment group is composed of at least 6 animals.

Animals were placed in an open field (60×50 cm) 5 minutes after i.v. administration of the treatment or 30 minutes after i.p. injection. Their locomotor activity was recorded using a video based computer system (ViewPoint, France). The following parameters were recorded for 3 minutes: the total distance, the time and the speed traveled by the animals. An additional parameter, rectal body temperature, was monitored at the end of the open-field examination using a thermostat thermometer (Cole Parker Model 8402-00) and a thermostat probe (YSI 400 Model 402).

Results are expressed as mean±SE and the differences between vehicle and treatments are compared by ANOVA, followed by post-hoc Tukey's test. A value of p<0.05 is considered to be statistically significant.

As far as the total distance traveled by the animals is concerned, fluctuations were observed between the treatment groups. Animals injected i.v. with vehicle traveled 1000 cm during the 3 minutes follow-up. Animals administered 0.1, 0.5 and 1 mg/kg of compound A displayed an increased locomotor activity with total distances covered between 1300 and 1400 cm. This increase was not statistically significant. Animals administered 1, 2, 3, 4, 5 and 6 mg/kg of compound R also displayed fluctuations in total distance covered and again this phenomenon had no statistical significance. Similarly, the average speed of vehicle treated animals is 5.9 sec/cm, while animals treated with 0.1, 0.5 and 1 mg/kg of compound A i.v. traveled at a slightly increased speed of 7 to 7.2 sec/cm. Animals treated with 1, 2, 3, 4, 5 and 6 mg/kg of compound R traveled at speeds ranging from 4 to 8 sec/cm in a dose independent manner. These differences in speed were not statistically significant in either direction. Finally, the mean rectal body temperature of vehicle treated animals was 38.4° C. Compound A and R induced a moderate hypothermia of about 2° C. that was statistically significant but in a range that has no severe physiological meaning. These experiments were repeated with i.p. route of administration and similar observations were made. The only difference between the two routes of administration is that the doses tested in i.p. injection were about an order of magnitude higher and compound A was tested up to 30 mg/kg i.p. while compound R was tested up to 40 mg/kg.

Though compounds A and R displayed some moderate side effects, these phenomena were observed at dosage well above their effective doses by at least 6-8 fold, and the compounds were overall well tolerated by the mice. No mortality was observed, therefore the maximal tolerated dose is well above 40 mg/kg i.p. The behavioral effects of the bicyclic CB2 ligands were monitored in a very short period following injection and proved to be not only moderate but also transient, since 24 hours after injection all animals were back to baseline behavior. It should be kept in mind that compounds of the invention bind preferably to the CB2 receptor but some retain binding capacity to the CB1 receptor, known to mediate such side effects. In this context it should be noted that compounds A and R bind to the CB1 receptor with an IC₅₀ of about 30-40 nM. Thus it can be assumed that compounds binding to the CB1 receptor with lowest affinity, as expressed by an IC₅₀ value for displacement of above 40 nM, will be even safer.

The effect of test compounds on overall locomotor ability was assessed in a second experimental setup, where the animals were submitted to a functional test using the rotarod apparatus as described by Rozas et al. (Rozas G. et al., J. of Neuroscience Methods 83: 165-75, 1998). The animals, male ICR mice (average body weight 40 g, Harlan, Israel), were trained for 4 days before beginning the experiment. Their task was to stay on the accelerating rod without falling for 12 minutes (3 minutes at each speed). The tested speeds were: 15, 19, 23 and 27 rpm. Animal performance on the rod was scored as follows: each animal could obtain a maximum of 3 points (1 for each minute) for full walking on the rod at each speed. Therefore, an animal could get a maximum score of 12 points (3 for each speed). Catching the circling beam of the rod without walking subtracted 0.5 points for every 3 circles circled by the animal. The first 3 circles did not affect the score. After proper training, at least 6 animals per group were administered i.p. with various doses of test compounds and controls, at volume dose of 5 ml/kg. Score was determined in the rotarod apparatus at time zero prior to compound injection and 30 minutes, 3 and 24 hours after compound or vehicle administration.

Results are expressed as mean±SD and the differences between vehicle and treatments are compared by ANOVA, followed by post-hoc Tukey's test. A value of p<0.05 is considered to be statistically significant. Vehicle treated animals displayed at all time point the maximal score of 12, since their locomotor ability was not affected whatsoever. Animals treated with 10 mg/kg of compound A displayed a statistically significant but transient decrease in locomotor activity with an average score of 5.6 thirty minutes after injection. This effect disappeared at later time points with average scores of 9.7 and 11.9 at 3 and 24 hours respectively. Such transient effect observed in the first half hour might be due to the CB1 binding ability of compound A, suggesting that compounds binding to the CB1 receptor with lowest affinity will be even safer. Based on these results, compounds were tested at time points at least half an hour after injection to ensure that the effects observed would be minimally affected, if at all, by residual CB1 binding ability.

### Example 18

### Effect of the Compounds on the Cardiovascular System

The purpose of this study was to assess the safety of the test compounds in male Sprague Dawley rats (270-350 g, Harlan, Israel). The test compounds were dissolved in vehicle, diluted 1:20 in sterile saline and injected either i.v. or i.p. at doses ranging from 0.1 to 2 mg/kg for i.v. administration or 10 to 40 mg/kg for i.p. administration, in volumes of 0.5 ml/100 g body weight. Each treatment group was composed of at least 6 animals. A cannula (PE 50, Clay Adams, USA) was implanted into the femoral artery under halothane anesthesia (induction 4% and maintenance 1%)). The vein was cannulated for drug administration. The arterial cannula was attached to a pressure transducer (Ohmeda DT-XX USA). The transducer was connected to a data acquisition system (Biopac, USA). Recordings of the heart rate (HR), mean arterial blood pressure (MABP) and electrocardiogram (ECG lead 2) were taken for 20 minutes before treatment, for the establishment of a stable baseline, up to 60 minutes following injections of the test compounds. Animals were also connected to a temperature recorder through a rectal thermistor probe (YSI Model 400, USA) and rectal temperature was monitored along the duration of the study. At the end of the study animals were euthanized by i.p. injection of 100 mg/kg sodium pentobarbitone.

Differences between vehicle and treatments are compared by one-way ANOVA, followed by post-hoc Newman-Keuls tests (Prism software from Graphpad, San Diego). A value of p<0.05 is considered to be statistically significant.

Vehicle treated animals exhibited no change in their blood pressure values. MABP remained stable at average values of about 100 mmHg. Compound A induced a dose related transient hypotension. At the lowest dose tested i.v., 0.1 mg/kg caused a decrease of only 4 mmHg at 5 minutes post injection, at later time points (15, 30, 45 and 60 minutes) MABP of treated animals returned to baseline. At the intermediate dose of 0.5 mg/kg of compound A, the decrease in MABP was of about 25 mmHg at 5 minutes, progressively increasing back to baseline range 30 minutes after injection, while at the highest dose of 1 mg/kg the decrease in MABP was of about 30 mmHg at 5 minutes, progressively increasing back to baseline range 45 minutes after injection. Similar results were obtained with compound R at 1 mg/kg. When injected i.p., compound A caused a moderated hypotension not exceeding 15 mmHg at up to 30 mg/kg, while compound R caused at most a reduction of 36 mmHg at 40 mg/kg. At none of the dose tested was the hypotension fatal, moreover this phenomenon was transient, during at most 45 minutes at the highest dose of compound A and R. If the hypotension was expressed by a decrease in MABP of over 50% or by a prolonged effect, the safety of the test compounds could have been questioned.

The heart rate displayed a very similar pattern with stable baseline values around 350 beats per minute for vehicle treated animals, while 0.1 mg/kg of compound A caused a minor insignificant and transient decline in HR and the higher doses caused a clearer and more prolonged decrease in HR. The maximal drop in HR was of about 80 beats per minute and did not last more than 15 minutes with a return to normal baseline values within at most 45 minutes since injection. Again, when compounds were injected i.p. the effect on HR was almost null at doses up to 20 mg/kg of compound A and up to 40 mg/kg of compound R. Twenty mg/kg of compound A caused a minor drop of only about 17 beats per minute over the one hour follow-up (5% from baseline), while 40 mg/kg of compound R caused minor fluctuations of 10% amplitude resulting in an average drop of only about 4 beats per minute. For comparison, the vehicle treated animals also displayed a minor drop in HR of about 17 beats/min representing 5% decrease as compared to baseline. If the effect on heart rate was expressed by a decrease in number of beats per minute of over 50% or by a prolonged effect, the safety of the test compounds could have been questioned.

Maximum tolerated dose was not reached during the course of this study and it can be assumed that in i.p. route of administration this value is higher than at least 40 mg/kg. Depending on the model used and the indication tested, the compounds of the invention displayed therapeutically significant activity in the mg/kg range of doses (from about 0.1 up to 10 mg/kg). The therapeutical range is thus well bellow the still unidentified toxic range, which is at least 4 folds higher. For example in EAE, 1 mg/kg i.p. of compound A caused a significant 35% reduction in clinical score AUC, in this case the therapeutic index is at least 40, while in paw edema 0.5 mg/kg i.p. of compound R caused a 31% decrease in paw thickness, in this case the therapeutic index is at least 80. It should be kept in mind that in these models, the test compounds' results were comparable in activity to commercially available drugs. Altogether these results support that bicyclic CB2 ligands of the invention are safe and potent alternatives to treat wide range of disease and disorders.

### Example 19

### Effect of Repeated Administration of Compounds on Development of Tolerance

One of the major problems encountered by the medical community when using morphine to treat severe pain conditions is the fact that, with time, patients develop tolerance to the drug. In order to maintain analgesic activity the dose of morphine can be at first gradually increased, but chronic use will ultimately reach a point of saturation where the drug cannot alleviate pain any longer. Tolerance to cannabinoids selective to the CB1 receptor might also develop. It was already proved in the studies described in examples 17 and 18 above, that the residual CB1 binding capacity of some of the bicyclic CB2 ligands caused no severe nor prolonged side effects and was overall well tolerated. To further strengthen the safe character of the compounds of the invention, their ability to induce tolerance was tested.

Tolerance was assessed in the tail flick model above described. Briefly, the test compounds were administered twice daily i.p. for up to 10 days to groups of 10 animals each. The noxious pain threshold was measured as previously described in example 13 thirty minutes after the first drug administration on days 1, 4, 8 and 10. Results are expressed as mean latency time till the animal flicked its tail±SE. The differences between the latency times or the % of animals showing analgesia among various treatment groups was analyzed by analysis of variance (ANOVA) followed by post-hoc Tukey's test (for latency) or Fisher's exact test (for % animals). A value of p<0.05 is considered to be statistically significant.

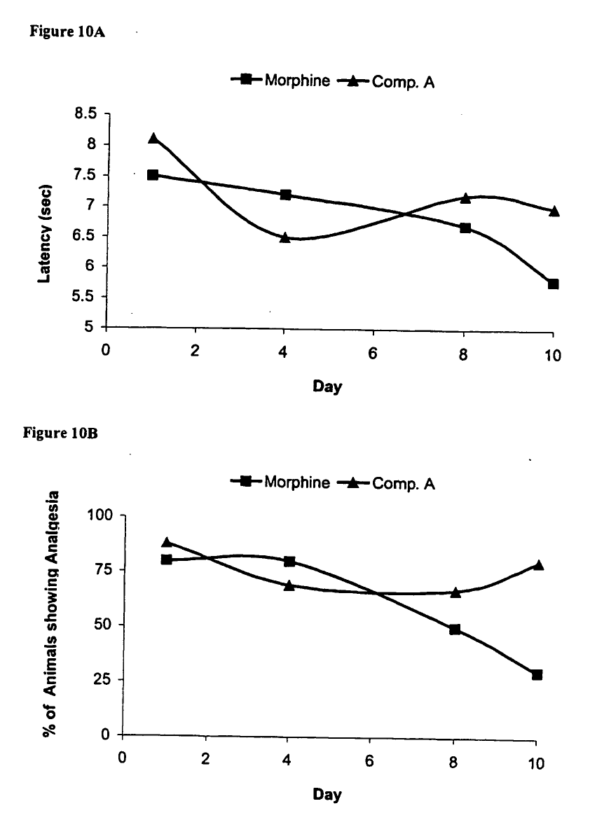

Results are depicted in FIG. 10, where panel A shows the impact of compound A as compared to morphine on the latency time and panel B shows the impact on the % of animals showing increased analgesia. Administration of 5 mg/kg morphine twice daily for 10 days caused the expected development of tolerance in the treated animals, as expressed by a gradual decrease both in the latency time and in the percent of animals with improved analgesia over the course of the study. The latency time was 7.5 sec on day one and only 5.8 sec on day 10, while the % of animals showing increased analgesia was 80% on day one and only 30% on day 10. On the other hand, the twice daily injections of 10 mg/kg compound A have no significant effect on these parameters, meaning that compound A administration for 20 times did not cause the development of tolerance at the dose previously shown to be therapeutically effective. Specifically, the latency time observed in animals treated with 10 mg/kg of compound A was 8.1 sec on day one and still as high as 7 sec on day 10, while the % of animals showing increased analgesia is 88% on day one and still 80% on day 10. Moreover, it should be noted that the rectal body temperature was assessed on day 10 and found to be within normal range for both treatment groups. Altogether, these studies proved than not only compounds of the invention are more effective than morphine in relieving pain, they are also safer since they did not induce tolerance.

### Example 20

### Diabetes Type I: the NOD Mice Model

The protective activity of bicyclic CB2 binding compounds in an experimental setup relevant to human insulin-dependent diabetes mellitus, is tested in the non-obese diabetic (NOD) mouse model.

NOD/At female mice (70-80 days old at study onset, Harlan, Israel) are weighted at day 1. Their baseline glucose level is established using a drop of blood obtained by sectioning the tip of the tail and a glucometer with the appropriate glucosticks (Elite, Bayer). Mice are then injected i.p. with cyclophosphamide (Sigma) diluted in saline at a dose of 300 mg/kg. The appearance of glucose in the urine of the animals is monitored every two days using a urine multistick (Bayer). When this test indicates that the animals reach glucourea, then the level of glucose in the blood is reassessed during two consecutive days after overnight starvation. Animals are defined as diabetic if their glucose blood levels are above 300 mg/dl. Three days following the diagnostic of diabetes, the animals are sacrificed by i.p. injection of 100 mg/kg pentobarbitone. Their spleen and pancreas are removed for further study including FACS analysis of the T cells subpopulations in the spleen and histo- and immuno-pathological evaluation of the pancreas.

The histopathological evaluation is performed on ten Langerhans islands for each animal and the scoring is according to the following method (Sempe P. et al., Eur. J. Immunol. 21: 1163-9, 1991). The severity of the damage is scored according to the level of mononuclear infiltrate: 0-no infiltration, 1-periductular infiltrate, 2-peri-islet infiltrate, 3-intra-islet infiltrate, 4-intra-islet infiltrate associated with β-cell destruction. The mean score for the pancreas of each animal is calculated by dividing the total score by the number of islets examined.

### Example 21

### Renal Ischemia

The nephro-protective activity of bicyclic CB2 binding compounds is tested in an acute renal ischemia model in rats.

Male Sprague Dawley rats (250 g average body weight, Harlan, Israel) are anesthetized with a combination of xylazine and pentobarbitone 8 and 35 mg/kg i.p. respectively. Then a 45-minutes ischemia is induced bilaterally on both kidneys. The sedated animals are positioned on their backs. The abdomen skin is shaved and cleaned with 70% ethanol. A midline skin incision is performed (2-3 cm long) and the abdomen is opened through an incision in the linea Alba. The kidneys are explored after gentle removal of the intestines to the opposite direction. While this is done, the intestines are covered with wet (warm saline 37° C.) sterile sponges. The renal arteries are isolated by blunt dissection from the surrounding fat, and occluded together with the renal veins in the kidney hilus by arterial micro clips (FST Canada). Kidneys that become pale immediately after artery occlusion are considered ischemic. Only animals showing that both kidneys are ischemic are included in the study. During the ischemic insult the intestines are returned into the abdominal cavity. The wound is covered with wet sponges (they were kept wet by rinsing warm saline). In addition, rectal temperature is monitored to remain between 37° C.-38° C. Rectal temperature is measured using a thermistor (YSI USA model 400) and a measuring unit (Cole Parmer model 8402-00).

Forty-five minutes after the ischemia initiation, the artery clips are removed. Reperfusion is verified by the return of the pink color of the kidney. The wound is then closed with 3-0 silk suture material (Assut, Switzerland) in two layers (abdomen wall and skin). At 1, 3 and 7 days post ischemic insult animals are lightly anesthetized in an anesthesia chamber with ether and blood samples are collected after an infra orbital sinus puncture. Blood is collected into eppendorf tubes, and centrifuged (4000 rpm for 5 minutes). Serum is then separated and kept at −20° C. prior to evaluation of blood levels of creatinine and blood urea nitrogen (BUN). At the end of the study, animals are euthanized with pentobarbitone sodium 100 mg/kg i.p. Kidneys are removed, weighted and kept in 4% formaldehyde solution for possible further usage.

Treatments are administered i.v. into the femoral vein at 5 ml/kg to 10 animals per group, immediately after the end of the ischemic insult. Results are compared to ischemic (vehicle treated) and sham (the same procedure, without renal artery occlusion).

The blood levels of BUN and creatinine are compared using ANOVA followed by Duncan's post-hoc test.

### Example 22

### The Langendorff Perfusion Model for Measuring Cardioprotection

Endogenous cannabinoids were recently shown to be involved in the cardioprotective effect of LPS against myocardial ischemia (Lagneux, C. & Lamontagne, D., Br. J. Pharmacol. 132: 793-6, 2001). The cardioprotective effect of the novel bicyclic compounds is tested in the Langendorff model of the isolated perfused rat heart. Male Sprague-Dawley rats weighing 280±20 g are used for perfusion experiments in compliance with the NIH Guide for the Care and Use of Laboratory Animals (NIH Publication No. 85-23, revised 1996). The animals are injected intraperitoneally with sodium heparin (500 U) and anesthetized with pentobarbital (30 mg/animal). Hearts are immediately removed and placed in heparinized ice-cold saline solution. The aorta is cannulated to a Langendorff perfusion apparatus and the pulmonary artery cut open to provide free drainage of effluent. Retrograde aortic perfusion is maintained with modified Krebs-Henseleit (KH) solution. The KH is aerated with a mixture of 95% oxygen and 5% carbon dioxide. Aortic perfusion is maintained at 37° C., at a constant pressure of 90 cm H₂O.

Short Term Ischemia at Normothermia.

Hearts undergo 20 min of KH perfusion, 25 min of no-flow global ischemia (at 37° C.), and 45 min of KH reperfusion. This has been shown to reduce the work index (LVDP×HR) recovery to ˜40%, a potential for improvement by drugs. Different concentrations of the compounds are added during pre-ischemic perfusion, reperfusion or both.

Hemodynamic Measurements.

A latex balloon-tipped catheter is inserted through a small cut in the left atrium and advanced through the mitral valve into the left ventricle. The balloon is connected, through a pressure transducer, to a recording system (Hewlett Packard 7758B, USA). The balloon is inflated and equilibrated to give an end-diastolic pressure of 0 mm Hg. Left ventricular systolic and diastolic pressures and time derivatives of pressure are measured during contraction (+dP/dt) and relaxation (−dP/dt). Left ventricular developed pressure (LVDP) is calculated from the difference between the systolic and diastolic pressures. The work index of the heart (LVDP×HR) is calculated from the product of LVDP and heart rate (HR). Coronary flow (CF) is measured by collecting the effluent drained through the pulmonary artery in the pre-ischemic period and during reperfusion. Hearts are excluded from the study if arrhythmias develop, thrombus forms, or LVDP and heart rate after the first 20 min of perfusion is less than 60 mm Hg, and 210 beat/min respectively.

Results are expressed as Mean±SEM. Statistical differences between groups of hearts are calculated using the ANOVA and Mann-Whitney rank tests A value of p<0.05 is considered to be statistically significant.

### Example 23

### Effect of Compounds on Tumor Cell Lines and Tumors

In Vitro.

Cells from several tumor-derived cell lines are tested for their proliferation capacity in presence of our test compounds. Tumor cell lines are obtained from ATCC and grown according to supplier recommendation. Cells are seeded in a 24 well plate (10⁵ cells/ml/well) and grown overnight. The cells are incubated with the test compounds (1-100 μM) or vehicle (0.1% DMSO final concentration). Cell viability is determined 24 hours later using standard crystal violet staining. The culture medium is removed from the wells and the cells are fixed by adding 1 ml/well of 2% formaldehyde in PBS for 10 minutes. Following fixation the cells are washed three times with PBS and 250 μl of 0.5% (w/v) crystal violet is added to each well and the plates are incubated for 30 minutes at room temperature with gentle agitation. The stained cells are then washed three times with double distilled water and the color is extracted by adding to each well 250 μl of 10% acetic acid. The plates are agitated for 15 minutes at room temperature and 100 μl are transferred in duplicate to a 96 well plate for reading. Optical density (OD) of the cells is measured at 620 nm in an ELISA reader and results are expressed as % viable cells. Absorbance of untreated cells is recorded as 100%. The IC₅₀ (dose inhibiting cell growth by 50%) is determined.

Moreover, the cells are stained for activated caspase 3 to determine whether they died through an apoptotic mechanism. The medium from the wells is discarded and cells are fixed by adding 1 ml of 4% formaldehyde in PBS, for 10 min. Cells are washed twice with PBS-0.1% Tween20 (PBS-T) and permeabilized with cold methanol for 20 min. The cells are washed twice with PBS-T and incubated with 1 ml blocking solution (3% BSA, PBS-T) for 30 min. The primary antibody (rabbit anti-cleaved caspase 3 (asp175) Cell Signaling Technology, diluted 1:50 with blocking solution) is added and the cells incubated for 60 min. at 37° C. The cells are washed twice with PBS-T. The secondary antibody (HRP conjugated anti-rabbit IgG diluted 1:200 with blocking solution) is added to the wells and incubated for 60 min. at RT. Cells are washed twice with PBS-T and incubated for 10 min with a fluorescein tyramide reagent (NEN, diluted 1:50 with amplification diluent). Cells are washed twice with PBS-T and the signal visualized by fluorescence or confocal microscope. Beside monitoring activated caspase-3, the expression of apoptosis-related genes in cells treated with dexanabinol and its analogs is compared to that in untreated cells. The procedure for real-time RT-PCR is as previously described. For each gene, a pair of specific PCR primers is designed and the reaction is done according to the ABI protocols. The quantification of level of expression of each gene is normalized to a housekeeping gene and compared to RNA samples from non-treated cells.

In Vivo.

Once we have selected the tumor cell lines whose proliferation is inhibited by the bicyclic CB2 binding test compound in vitro, we test the efficiency in vivo. Cells are grown according to supplier recommendation. Predetermined amounts (1×10⁶ cells in constant volume of 0.12-ml/animal) are injected s.c. above the right femoral joint in nude CD-1 male mice (average weight 20-25 g, Harlan, Israel). Each treatment group is composed of at least 7 animals. Each animal is clinically monitored daily. The growth of the tumor is also monitored during the daily visits but actual measurements are recorded once a week. When tumors reach the appropriate size, animals are treated with either vehicle, 5 ml/kg/day, or with our test compounds, in the range of 2.5 to 10 mg/kg/day.

### Example 24

### Effect of the Compounds in the Model for Inflammatory Bowel Disease

The anti-inflammatory activity of bicyclic CB2 binding compounds is tested in a masked study of acetic acid-induced IBD in rats.

Male Sprague Dawley rats (10 weeks old, 200-250 g, Harlan, Israel) are lightly anaesthetized by i.p. injection of a ketamine:rompun combination (100:10 mg/kg respectively). A polyethylene catheter (outer diameter 1.7 mm) is inserted through the rectum 5 cm into the colon. And 2 ml of 5% acetic acid are then slowly administered into the colon. Fifteen seconds later the colon is washed with 3 ml saline and 15 seconds later with additional 3 ml of saline. Immediately after, each group of animals is treated with either one of the appropriate treatments. All treatments are administered once daily for 7 days. Animals are clinically followed for 1 week. During this period, the following parameters were daily monitored and recorded: body weight, presence of blood in the stool and stool consistency. These findings are scored according to table 1.

TABLE 1
Criteria for Scoring Disease Activity Index (DAI^{#}) of IBD
(Murthy S. N. et al., Dig. Dis. Sci. 38: 1722-34, 1993).

| | | | Occult Blood or |
|---|---|---|---|
| Score | Weight Loss (%) | Stool Consistency* | Gross Bleeding |
| 0 | None | Normal | Negative |
| 1 | 1-5 | Loose Stool | Negative |
| 2 | 5-10 | Loose Stool | Hemoccult Positive |
| 3 | 10-15 | Diarrhea | Hemoccult Positive |
| 4 | >15 | Diarrhea | Gross Bleeding |

^{#}DAI - (combined score of weight loss, stool consistency, and bleeding)/3.
*Normal stool - well formed pellets; loose stools - pasty stool that does not stick to the anus; and diarrhea - liquid stools that sticks to the anus.

Seven days post disease induction animals are sacrificed with pentobarbital 100 mg/kg i.p. The whole colon is excised, slit longitudinally and examined under a magnifying glass, and any visible damage is recorded and scored according to table 2.

TABLE 2
Gross Pathology Scoring Method for Evaluating the Severity of IBD
(Wong et al., J. Pharm. Exp. Ther. 274: 475-80, 1995).

| Score | Pathology |
|---|---|
| 0 | No damage |
| 1 | Localized hyperemia and/or edema |
| 2 | Two or sites of hyperemia and/or edema |
| 3 | Localized erosion |
| 4 | Localized ulcer |
| 5 | More then 1 site of erosion/or ulcer, or 1 erosion site or ulcer |
| | extending >2 cm along the length of the colon |

The clinical outcome is analyzed using analysis of variance (ANOVA) followed by Duncan's post-hoc test. A non-parametric test (Wilcoxon Rank Sum Test) is used for evaluating the gross pathology findings.

### Formulation Examples

Lyophilized Powder for Reconstitution.

As noted above, some of the compounds of the invention are highly lipophilic with calculated logP above 5, rendering them rather insoluble in water. Though these compounds can be formulated in a variety of compositions that accommodate their lipophilic nature, approaches based on chemical modification of the parent compounds have been employed to improve water solubility thus enlarging the range of formulations and routes of administration adapted for said compounds. One such example is compound R, which is the hemisuccinate derivative of compound A. This esterification step improves dramatically the calculated logD of the compound at pH 7. Compound A has a logD of 6.21 while its hemisuccinate derivative compound R has a logD of only 3.76 (calculated using ACD software). In terms of water solubility, it means that at neutral pH compound A is expected to dissolve in water at a concentration of 7.6×10⁻⁵ g/l while compound R is expected to be soluble up to 0.024 g/l. The improved solubility opened the road to alternative formulation such as the preparation of lyophilized powder for reconstitution.

Thirty milligrams of compound R were dissolved in 0.3 ml tert-butanol. To obtain a final 5 mg/ml drug concentration, 6 ml of phosphate buffer (NaH₂PO₄ and Na₂HPO₄, pH 7.8, 80 mM) were added. Then 150 mg lactose were added to get a final lactose concentration of 25 mg/ml. With dissolution of the compound the pH of the solution tended to decrease and it was readjusted to pH 7.8 using 0.2 N NaOH. The resulting solution was freeze-dryed overnight to get a lyophilized powder. The lyophilized powder of compound R was later reconstituted with water to get a clear solution of the ester derivative in the range of about 5 mg/ml final concentration, which is an unexpected dramatic increase in solubility as compared to the initial 76 ng/ml of compound A parent drug. The same formulation was also prepared containing, in addition to the phosphate buffer, benzyl alcohol at a concentration of 9 mg/ml. Sucrose 5%, or mannitol 5%, or glycerol 2%, or dextran 5%, or up to 5%, preferably 1-2.5%, polyvinylpyrrolidone (PVP) K-30, or PVP K-10 can be added instead of lactose as diluent and cryoprotectants during the freeze-drying process. Lyophilized compound R was reconstituted with sterile water for irrigation and was shown to be stable for at least up to two hours, as monitored by HPLC. During this period of time up to 14% of compound R hydrolyzed to parent compound A. As above-reported, compound R is biologically active when formulated in CREMOPHOR EL®:ethanol. Preliminary studies indicate that reconstituted lyophilized compound R also achieves the therapeutical goal of the compound. For instance, compound R formulated in cosolvent yielded a maximal reduction in paw edema of 31% at about 1 M/kg while reconstituted lyophilized compound R yielded in the same model 29.5% reduction in paw edema at about 0.5 μM/kg. This study shows that pharmaceutically acceptable salts or esters derivatives can be prepared for the compound of the invention, thus allowing the preparation of various types of formulations and the administration of such compound by various routes to treat the diseases induced in the models above-described.

Although the present invention has been described with respect to various specific embodiments presented thereof for the sake of illustration only, such specifically disclosed embodiments should not be considered limiting. Many other such embodiments will occur to those skilled in the art based upon applicants' disclosure herein, and applicants propose to be bound only by the spirit and scope of their invention as defined in the appended claims.

1. A compound of the general formula (I):
Formula I 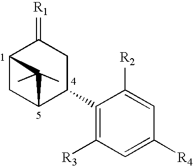
having a specific stereochemistry wherein C-5 is S, the protons at C-1 and C-5 are cis in relation to one another and the protons at C-4 and C-5 are trans; and wherein:
R₁ is selected from the group consisting of
(a) O or S,
(b) C(R′)₂ wherein R′ at each occurrence is independently selected from the group consisting of hydrogen, cyano, —OR″, —N(R″)₂, a saturated or unsaturated, linear, branched or cyclic C₁-C₆ alkyl, C₁-C₆ alkyl-OR″ or C₁-C₆ alkyl-N(R″)₂ wherein at each occurrence R″ is independently selected from the group consisting of hydrogen, C(O)R′″, C(O)N(R′″)₂, C(S)R′″, saturated or unsaturated, linear, branched or cyclic C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂ alkyl, and
(c) NR″ or N—OR″ wherein R″ is as previously defined;
R₂ and R₃ are each independently selected from the group consisting of
(a) halogen,
(b) —R″, —OR″, —N(R″)₂, —SR″, —S(O)(O)NR″, wherein at each occurrence R″ is as previously defined,
(c) —S(O)R^{b}, —S(O)(O)R^{b}, —S(O)(O)OR^{b} wherein R^{b} is selected from the group consisting of hydrogen, saturated or unsaturated, linear, branched or cyclic C₁-C₆ alkyl, C₁-C₆ alkyl-OR″, and C₁-C₆ alkyl-N(R″)₂, wherein R″ is as previously defined, and
(d) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear, branched or cyclic C₁-C₆ alkyl and R^{e} is at each occurrence selected from the group consisting of hydrogen and R^{d} as previously defined; and
R₄ is selected from the group consisting of
(a) R wherein R is selected from the group consisting of hydrogen, halogen, OR′″, OC(O)R′″, C(O)OR′″, C(O)R′″, OC(O)OR′″, CN, NO₂, N(R′″)₂, NC(O)R′″, NC(O)OR′″, C(O)N(R′″)₂, NC(O)N(R′″)₂, SR′″, and C(S)R′″, wherein at each occurrence R′″ is as previously defined,
(b) a saturated or unsaturated, linear, branched or cyclic C₁-C₁₂ alkyl-R wherein R is as previously defined,
(c) an aromatic ring which can be further substituted at any position by R wherein R is as previously defined, and
(d) a saturated or unsaturated, linear, branched or cyclic C₁-C₁₂ alkyl optionally terminated by an aromatic ring which can be further substituted as defined in (c);
with the proviso that when R₁ is O and R₂ and R₃ are OH, then R₄ is other than a straight or branched C₅-C₁₀ alkyl, C₅-C₁₀ alkenyl, C₅-C₈ cycloalkyl and C₅-C₈ cycloalkenyl;
and pharmaceutically acceptable salts, esters or solvates thereof.

2. The compound of claim 1 wherein R₁ is O, CH₂ or N—OH, R₂ and R₃ are each independently H, OH, OCH₃, succinate, fumarate or diethylphosphate, and R₄ is 1,1-dimethyl-pentyl, 1,1-dimethyl-heptyl, 1,1-dimethyl-pent-4-enyl, 1,1-dimethyl-hept-6-ynyl, 1,1-dimethyl-3-phenyl-propyl, 1,1-dimethyl-5-bromo-pentyl, 1,1-dimethyl-5-cyano-pentyl, 1,1,3-trimethyl-butyl, 1-methyl-1-p-chlorophenyl-ethyl, or 1-ethyl-1-methyl-propyl, with the proviso defined for formula (I).

3. The compound of claim 1 wherein R₁ is O, R₂ and R₃ are OH and R₄ is 1,1-dimethyl-3-phenyl-propyl, 1,1-dimethyl-hept-6ynyl, 1,1-dimethyl-5-bromo-pentyl, 1,1-dimethyl-5-cyano-pentyl, or 1-methyl-1-p-chlorophenyl-ethyl.

4. The compound of claim 1 wherein R₁ is O, R₄ is 1,1-dimethyl-heptyl, and R₂ and R₃ are both H, OCH₃, diethylphosphate or succinate.

5. The compound of claim 1 wherein R₁ is O, R₄ is 1,1-dimethyl-heptyl, R₂ is OH and R₃ is OCH₃, diethylphosphate, fumarate or succinate.

6. The compound of claim 1 wherein R₁ is O, R₂ is succinate, R₃ is OH and R₄ is 1,1-dimethyl-pentyl.

7. The compound of claim 1 wherein R₁ is CH₂, R₄ is 1,1-dimethyl-heptyl, R₂ and R₃ are both OCH₃ or diethylphosphate.

8. The compound of claim 1 wherein R₁ is NOH, R₂ and R₃ are OH and R₄ is 1,1-dimethyl-heptyl.

9. A compound of the general formula (II): 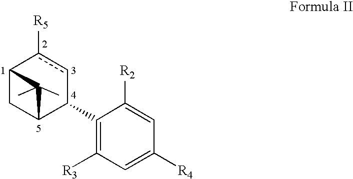
having a specific stereochemistry wherein C-5 is S, the protons at C-1 and C-5 are cis in relation to one another, the protons at C-4 and C-5 are trans, and C-2—C-3 is an optional double bond; and wherein:
R₅ is selected from the group consisting of
(a) halogen or hydrogen,
(b) —OR″, —N(R″)₂, —SR″, —S(O)(O)NR″, wherein at each occurrence R″ is independently selected from the group consisting of hydrogen, C(O)R′″, C(O)N(R′″)₂, C(S)R′″, saturated or unsaturated, linear, branched or cyclic C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂ alkyl,
(c) a saturated or unsaturated, linear, branched or cyclic C₁-C₆ alkyl-SR″ or C₁-C₆ alkyl-S(O)(O)NR″, wherein R″ as previously defined,
(d) —S(O)R^{b}, —S(O)(O)R^{b}, —S(O)(O)OR^{b} wherein R^{b} is selected from the group consisting of hydrogen, saturated or unsaturated, linear, branched or cyclic C₁-C₆ alkyl, C₁-C₆ alkyl-OR″, and C₁-C₆ alkyl-N(R″)₂, wherein at each occurrence R″ is as previously defined,
(e) a saturated or unsaturated, linear, branched or cyclic C₁-C₆ alkyl-S(O)R^{b}, C₁-C₆ alkyl-S(O)(O)R^{b}, C₁-C₆ alkyl-S(O)(O)OR^{b} wherein R^{b} is as previously defined, and
(f) R^{c} wherein R^{c} is selected from the group consisting of saturated or unsaturated, linear, branched or cyclic C₁-C₆ alkyl, C₁-C₆ alkyl-OR″, C₁-C₆ alkyl-N(R″)₂, C₁-C₆ alkyl-C(O)OR″, and C₁-C₆ alkyl-C(O)N(R″)₂ wherein at each occurrence R″ is as previously defined;
R₂ and R₃ are each independently selected from the group consisting of
(a) halogen,
(b) —R″, —OR″, —N(R″)₂, —SR″, —S(O)(O)NR″, wherein at each occurrence R″ is as previously defined,
(c) —S(O)R^{b}, —S(O)(O)R^{b}, —S(O)(O)OR^{b} wherein R^{b} is as previously defined, and
(d) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear, branched or cyclic C₁-C₆ alkyl and R^{e} is at each occurrence selected from the group consisting of hydrogen and R^{d} as previously defined; and
R₄ is selected from the group consisting of
(a) R wherein R is selected from the group consisting of hydrogen, halogen, OR′″, OC(O)R′″, C(O)OR′″, C(O)R′″, OC(O)OR′″, CN, NO₂, N(R′″)₂, NC(O)R′″, NC(O)OR′″, C(O)N(R′″)₂, NC(O)N(R′″)₂, SR′″, and C(S)R′″, wherein at each occurrence R′″ is as previously defined,
(b) a saturated or unsaturated, linear, branched or cyclic C₁-C₁₂ alkyl-R wherein R is as previously defined,
(c) an aromatic ring which can be further substituted at any position by R wherein R is as previously defined, and
(d) a saturated or unsaturated, linear, branched or cyclic C₁-C₁₂ alkyl optionally terminated by an aromatic ring which can be further substituted as defined in (c);
with the proviso that when R₅ is R^{c}, then R₄ is other than a straight or branched saturated C₁-C₁₂ alkyl chain, a straight or branched saturated —O—C₂-C₉ alkoxy chain optionally substituted at the terminal carbon by a phenyl group, and a straight or branched saturated C₁-C₇ alkyl chain terminated by a hydroxyl or by a straight or branched saturated —O—C₁-C₅ alkoxy chain;
and pharmaceutically acceptable salts, esters or solvates thereof.

10. The compound of claim 9 wherein R₅ is CH₂OC(O)C(CH₃)₃, OH or CH₃, R₂ and R₃ are each independently H, OH, or diethylphosphate, R₄ is CH₂OC(O)(CH₂)₃CH₃, 1,1-dimethyl-heptyl, 1,1-dimethyl-ethyl-phenyl, or 1,1-dimethyl-hept-6-ynyl, and there is an optional double bond between C-2 and C-3, with the proviso defined for formula (II).

11. The compound of claim 9 wherein R₅ is OH, R₄ is 1,1-dimethyl-heptyl, R₂ and R₃ are both H, OH, or diethylphosphate, and there is a single bond between C-2 and C-3.

12. The compound of claim 9 wherein R₅ is CH₃, R₂ and R₃ are OH, R₄ is 1,1-dimethyl-hept-6-ynyl, 1,1-dimethyl-ethyl-phenyl or CH₂OC(O)(CH₂)₃CH₃, and there is a double bond between C-2 and C-3.

13. The compound of claim 9 wherein R₅ is CH₂OC(O)C(CH₃)₃, R₂ and R₃ are OH, R₄ is 1,1-dimethyl-pentyl, and there is a double bond between C-2 and C-3.

14. A pharmaceutical composition comprising as an active ingredient a compound of the general formula (III): 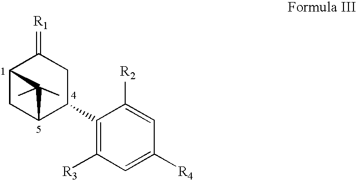
having a specific stereochemistry wherein C-5 is S, the protons at C-1 and C-5 are cis in relation to one another and the protons at C-4 and C-5 are trans; and wherein:
R₁ is selected from the group consisting of
(a) O or S,
(b) C(R′)₂ wherein R′ at each occurrence is independently selected from the group consisting of hydrogen, cyano, —OR″, —N(R″)₂, a saturated or unsaturated, linear, branched or cyclic C₁-C₆ alkyl, C₁-C₆ alkyl-OR″ or C₁-C₆ alkyl-N(R″)₂ wherein at each occurrence R″ is independently selected from the group consisting of hydrogen, C(O)R′″, C(O)N(R′″)₂, C(S)R′″, saturated or unsaturated, linear, branched or cyclic C₁-C₆ alkyl, C₁-C₆ alkyl-OR′″, and C₁-C₆ alkyl-N(R′″)₂, wherein at each occurrence R′″is independently selected from the group consisting of hydrogen or saturated or unsaturated, linear, branched or cyclic C₁-C₁₂ alkyl, and
(c) NR″ or N—OR″ wherein R″ is as previously defined;
R₂ and R₃ are each independently selected from the group consisting of
(a) halogen,
(b) —R″, —OR″, —N(R″)₂, —SR″, —S(O)(O)NR″, wherein at each occurrence R″ is as previously defined,
(c) —S(O)R^{b} , —S(O)(O)R^{b}, —S(O)(O)OR^{b} wherein R^{b} is selected from the group consisting of hydrogen, saturated or unsaturated, linear, branched or cyclic C₁-C₆ alkyl, C₁-C₆ alkyl-OR″, and C₁-C₆ alkyl-N(R″)₂, wherein R″ is as previously defined, and
(d) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear, branched or cyclic C₁-C₆ alkyl and R^{e} is at each occurrence selected from the group consisting of hydrogen and R^{d} as previously defined; and
R₄ is selected from the group consisting of
(a) R wherein R is selected from the group consisting of hydrogen, halogen, OR′″, OC(O)R′″, C(O)OR′″, C(O)R′″, OC(O)OR′″, CN, NO₂, N(R′″)₂, NC(O)R′″, NC(O)OR′″, C(O)N(R′″)₂, NC(O)N(R′″)₂, SR′″, and C(S)R′″, wherein at each occurrence R′″ is as previously defined,
(b) a saturated or unsaturated, linear, branched or cyclic C₁-C₁₂ alkyl-R wherein R is as previously defined,
(c) an aromatic ring which can be further substituted at any position by R wherein R is as previously defined, and
(d) a saturated or unsaturated, linear, branched or cyclic C₁-C₁₂ alkyl optionally terminated by an aromatic ring which can be further substituted as defined in (c);
and further comprising a pharmaceutically acceptable diluent or carrier.

15. The pharmaceutical composition of claim 14 wherein R₁ is O, CH₂ or N—OH, R₂ and R₃ are each independently H, OH, OCH₃, succinate, fumarate or diethylphosphate, and R₄ is 1,1-dimethyl-pentyl, 1,1-dimethyl-heptyl, 1,1-dimethyl-pent-4-enyl, 1,1-dimethyl-hept-6-ynyl, 1,1-dimethyl-3-phenyl-propyl, 1,1-dimethyl-5-bromo-pentyl, 1,1-dimethyl-5-cyano-pentyl, 1,1,3-trimethyl-butyl, 1-methyl-1-p-chlorophenyl-ethyl, or 1-ethyl-1-methyl-propyl.

16. The pharmaceutical composition of claim 14 wherein R₁ is O, R₂ and R₃ are OH and R₄ is 1,1-dimethyl-pentyl, 1,1-dimethyl-heptyl, 1,1-dimethyl-pent-4-enyl, 1,1-dimethyl-3-phenyl-propyl, 1,1-dimethyl-hept-6-ynyl, 1,1-dimethyl-5-bromo-pentyl, 1,1-dimethyl-5-cyano-pentyl, 1,1,3-trimethyl-butyl, 1-methyl-1-p-chlorophenyl-ethyl, or 1-ethyl-1-methyl-propyl.

17. The pharmaceutical composition of claim 14 wherein R₁ is O, R₄ is 1,1-dimethyl-heptyl, and R₂ and R₃ are both H, OCH₃, diethylphosphate or succinate.

18. The pharmaceutical composition of claim 14 wherein R₁ is O, R₄ is 1,1-dimethyl-heptyl, R₂ is OH and R₃ is OCH₃, diethylphosphate, fumarate or succinate.

19. The pharmaceutical composition of claim 14 wherein R₁ is O, R₂ is succinate, R₃ is OH and R₄ is 1,1-dimethyl-pentyl.

20. The pharmaceutical composition of claim 14 wherein R₁ is CH₂, R₄ is 1,1-dimethyl-heptyl, R₂ and R₃ are both OCH₃ or diethylphosphate.

21. The pharmaceutical composition of claim 14 wherein R₁ is NOH, R₂ and R₃ are OH and R₄ is 1,1-dimethyl-heptyl.

22. A pharmaceutical composition comprising as an active ingredient a compound of the general formula (II): 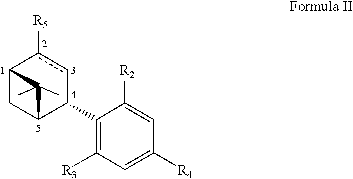
having a specific stereochemistry wherein C-5 is S, the protons at C-1 and C-5 are cis in relation to one another, the protons at C-4 and C-5 are trans, and C-2—C-3 is an optional double bond; and wherein:
R₅ is selected from the group consisting of
(a) halogen or hydrogen,
(b) —OR″, —N(R″)₂, —SR″, —S(O)(O)NR″, wherein at each occurrence R″ is independently selected from the group consisting of hydrogen, C(O)R′″, C(O)N(R′″)₂, C(S)R′″, saturated or unsaturated, linear, branched or cyclic C₁-C₆ alkyl, C₁-C₆ alkyl-OR′″, and C₁-C₆ alkyl-N(R′″)₂, wherein at each occurrence R′″ is independently selected from the group consisting of hydrogen or saturated or unsaturated, linear, branched or cyclic C₁-C₁₂ alkyl,
(c) a saturated or unsaturated, linear, branched or cyclic C₁-C₆ alkyl-SR″ or C₁-C₆ alkyl-S(O)(O)NR″, wherein R″ as previously defined,
(d) —S(O)R^{b}, —S(O)(O)R^{b}, —S(O)(O)OR^{b} wherein R^{b} is selected from the group consisting of hydrogen, saturated or unsaturated, linear, branched or cyclic C₁-C₆ alkyl, C₁-C₆ alkyl-OR″, and C₁-C₆ alkyl-N(R″)₂, wherein at each occurrence R″ is as previously defined,
(e) a saturated or unsaturated, linear, branched or cyclic C₁-C₆ alkyl-S(O)R^{b}, C₁-C₆ alkyl-S(O)(O)R^{b}, C₁-C6 alkyl-S(O)(O)OR^{b} wherein R^{b} is as previously defined, and
(f) —R^{c} wherein R^{c} is selected from the group consisting of saturated or unsaturated, linear, branched or cyclic C₁-C₆ alkyl, C₁-C₆ alkyl-OR″, C₁-C₆ alkyl-N(R″)₂, C₁-C₆ alkyl-C(O)OR″, and C₁-C₆ alkyl-C(O)N(R″)₂ wherein at each occurrence R″ is as previously defined;
R₂ and R₃ are each independently selected from the group consisting of
(a) halogen,
(b) —R″, —OR″, —N(R″)₂, —SR″, —S(O)(O)NR″, wherein at each occurrence R″ is as previously defined,
(c) —S(O)R^{b}, —S(O)(O)R^{b}, —S(O)(O)OR^{b} wherein R^{b} is as previously defined, and
(d) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear, branched or cyclic C₁-C₆ alkyl and R^{e} is at each occurrence selected from the group consisting of hydrogen and R^{d} as previously defined; and
R₄ is selected from the group consisting of
(a) R wherein R is selected from the group consisting of hydrogen, halogen, OR′″, OC(O)R′″, C(O)OR′″, C(O)R′″, OC(O)OR′″, CN, NO₂, N(R′″)₂, NC(O)R′″, NC(O)OR′″, C(O)N(R′″)₂, NC(O)N(R′″)₂, SR′″, and C(S)R′″, wherein at each occurrence R′″ is as previously defined,
(b) a saturated or unsaturated, linear, branched or cyclic C₁-C₁₂ alkyl-R wherein R is as previously defined,
(c) an aromatic ring which can be further substituted at any position by R wherein R is as previously defined, and
(d) a saturated or unsaturated, linear, branched or cyclic C₁-C₁₂ alkyl optionally terminated by an aromatic ring which can be further substituted as defined in (c); with the proviso that when R₅ is R^{C}, then R₄ is other than a straight or branched saturated C₁-C₁₂ alkyl chain, a straight or branched saturated —O—C₂-C₉ alkoxy chain optionally substituted at the terminal carbon by a phenyl group, and a straight or branched saturated C₁-C₇ alkyl chain terminated by a hydroxyl or by a straight or branched saturated —O—C₁-C₅ alkoxy chain;
and further comprising a pharmaceutically acceptable diluent or carrier.

23. The pharmaceutical composition of claim 22 wherein R₅ is CH₂OC(O)C(CH₃)₃, OH or CH₃, R₂ and R₃ are each independently H, OH, or diethylphosphate, R₄ is CH₂OC(O)(CH₂)₃CH₃,1,1-dimethyl-heptyl, 1,1-dimethyl-ethyl-phenyl, or 1,1-dimethyl-hept-6 ynyl, and there is an optional double bond between C-2 and C-3, with the proviso defined for formula (II).

24. The pharmaceutical composition of claim 22, wherein R₅ is OH, R₄ is 1,1-dimethyl-heptyl, R₂ and R₃ are both H, OH, or diethylphosphate, and there is a single bond between C-2 and C-3.

25. The pharmaceutical composition of claim 22, wherein R₅ is CH₃, R₂ and R₃ are OH, R₄ is 1,1-dimethyl-hept-6-ynyl, 1,1-dimethyl-ethyl-phenyl or CH₂OC(O)(CH₂)₃CH₃, and there is a double bond between C-2 and C-3.

26. The pharmaceutical composition of claim 22, wherein R₅ is CH₂OC(O)C(CH₃) ₃, R₂ and R₃ are OH, R₄ is 1,1-dimethyl-pentyl, and there is a double bond between C-2 and C-3.

27. The pharmaceutical composition according to claim 14, wherein the diluent comprises an aqueous cosolvent solution comprising a pharmaceutically acceptable cosolvent, a micellar solution or emulsion prepared with natural or synthetic ionic or non-ionic surfactants, or a combination of such cosolvent and micellar or emulsion solutions.

28. The pharmaceutical composition according to claim 27 wherein the diluent comprises a solution of ethanol, a surfactant and water.

29. The pharmaceutical composition according to claim 27 wherein the diluent is an emulsion comprising triglycerides, lecithin, glycerol, an emulsifier, and water.

30. The pharmaceutical composition according claim 14, in unit dosage form.

31. The pharmaceutical composition according to claim 30 suitable for oral administration.

32. The pharmaceutical composition according to claim 30 suitable for parenteral administration.

33. The pharmaceutical composition according to claim 22, wherein the diluent comprises an aqueous cosolvent solution comprising a pharmaceutically acceptable cosolvent, a micellar solution or emulsion prepared with natural or synthetic ionic or non-ionic surfactants, or a combination of such cosolvent and micellar or emulsion solutions.

34. The pharmaceutical composition according to claim 33 wherein the diluent comprises a solution of ethanol, a surfactant and water.

35. The pharmaceutical composition according to claim 33 wherein the diluent is an emulsion comprising triglycerides, lecithin, glycerol, an emulsifier, and water.

36. The pharmaceutical composition according claim 22, in unit dosage form.

37. The pharmaceutical composition according to claim 36, suitable for oral administration.

38. The pharmaceutical composition according to claim 36, suitable for parenteral administration.

39. A method for preventing, alleviating or treating a disease or disorder amenable to CB2 receptor modulation, by administering to an individual in need thereof of a therapeutically effective amount of a pharmaceutical composition according to claim 14.

40. A method for preventing, alleviating or treating autoimmune disease and inflammation, rheumatoid arthritis, multiple sclerosis, systemic lupus erythematosus, myasthenia gravis, diabetes mellitus type I, hepatitis, psoriasis, inflammatory bowel disease, tissue rejection in organ transplants, malabsorption syndromes, celiac disease, pulmonary disease, asthma and Sjgren's syndrome, by administering to an individual in need thereof of a therapeutically effective amount of a pharmaceutical composition according to claim 14.

41. A method for preventing, alleviating or treating neurological disorders, stroke, migraine, cluster headache, neurodegenerative diseases, Parkinson's disease, Alzheimer's disease, amyotrophic lateral sclerosis, Huntington's chorea, prion-associated diseases, poisoning of the central nervous system, and muscle spasm and tremor, by administering to an individual in need thereof of a therapeutically effective amount of a pharmaceutical composition according to claim 14.

42. A method for preventing, alleviating or treating pain including peripheral, visceral, neuropathic, inflammatory and referred pain, by administering to an individual in need thereof of a therapeutically effective amount of a pharmaceutical composition according to claim 14.

43. A method for preventing, alleviating or treating cardiovascular disorders, arrhythmia, hypertension and myocardial ischemic damage, by administering to an individual in need thereof of a therapeutically effective amount of a pharmaceutical composition according to claim 14.

44. A method for preventing, alleviating or treating cancer, by administering to an individual in need thereof of a therapeutically effective amount of a pharmaceutical composition according to any one of claim 14.

45. A method for preventing, alleviating or treating a disease or disorder amenable to CB2 receptor modulation, by administering to an individual in need thereof of a therapeutically effective amount of a pharmaceutical composition according to claim 22.

46. A method for preventing, alleviating or treating autoimmune disease and inflammation, rheumatoid arthritis, multiple sclerosis, systemic lupus erythematosus, myasthenia gravis, diabetes mellitus type I, hepatitis, psoriasis, inflammatory bowel disease, tissue rejection in organ transplants, malabsorption syndromes, celiac disease, pulmonary disease, asthma and Sjgren's syndrome, by administering to an individual in need thereof of a therapeutically effective amount of a pharmaceutical composition according to claim 22.

47. A method for preventing, alleviating or treating neurological disorders, stroke, migraine, cluster headache, neurodegenerative diseases, Parkinson's disease, Alzheimer's disease, amyotrophic lateral sclerosis, Huntington's chorea, prion-associated diseases, poisoning of the central nervous system, and muscle spasm and tremor, by administering to an individual in need thereof of a therapeutically effective amount of a pharmaceutical composition according claim 22.

48. A method for preventing, alleviating or treating pain including peripheral, visceral, neuropathic, inflammatory and referred pain, by administering to an individual in need thereof of a therapeutically effective amount of a pharmaceutical composition according to claim 22.

49. A method for preventing, alleviating or treating cardiovascular disorders, arrhythmia, hypertension and myocardial ischemic damage, by administering to an individual in need thereof of a therapeutically effective amount of a pharmaceutical composition according to claim 22.

50. A method for preventing, alleviating or treating cancer, by administering to an individual in need thereof of a therapeutically effective amount of a pharmaceutical composition according to claim 22.

51. A method for preventing, alleviating or treating neuropathic pain, by administering to an individual in need thereof of a therapeutically effective amount of a pharmaceutical composition comprising as an active ingredient (+){4-[4-(1,1-dimethylheptyl)-2,6-dimethoxy-phenyl]-6,6-dimethyl-bicyclo [3.1.1]hept-2-en-2-yl}-methanol.

52. A method for preventing, alleviating or treating Parkinson's disease, by administering to an individual in need thereof of a therapeutically effective amount of a pharmaceutical composition comprising as an active ingredient (+) {4-[4-(1,1-dimethylheptyl)-2,6-dimethoxy-phenyl]-6,6-dimethyl-bicyclo[3.1.1]hept-2-en-2-yl}-methanol.
